(19) United States
(12) Patent Application Publication
(10) Pub. No.: US 2020/0335191 A1
(43) Pub. Date: Oct. 22, 2020

(54) Electronic Devices and Methods for Treatment of Depressive Symptoms, Depressive Disorders Utilizing Digital Therapies

(71) Applicants: Pear Therapeutics, Inc., Boston, MA (US); Novartis AG, Basel (CH)

(72) Inventors: Michael Brown, Arlington, MA (US); Brent Paul Kersanske, Stoneham, MA (US); Kenneth R. Weingardt, South San Francisco, CA (US); Jillian Christine Ahrens, Kapolei, HI (US); Seyyed Nassir Ghaemi, Cambridge, MA (US); Alexandra Kubara, Basel (CH); Florian von Raison, Basel (CH)

(73) Assignees: Pear Therapeutics, Inc., Boston, MA (US); Novartis AG, Basel (CH)

(21) Appl. No.: 16/846,996

(22) Filed: Apr. 13, 2020

Related U.S. Application Data
(60) Provisional application No. 62/835,295, filed on Apr. 17, 2019.

(30) Foreign Application Priority Data
May 24, 2019 (DK) PA 2019 70330

(51) Int. Cl. G16H 20/10; G16H 20/70

(52) U.S. Cl. CPC G16H 20/10; G16H 20/70

(57) ABSTRACT

The present disclosure relates to uses of computerized behavioral therapy in the treatment of depressive symptoms or depressive disorder associated with multiple sclerosis.

### CROSS REFERENCE TO RELATED APPLICATIONS

This U.S. patent application claims priority under 35 U.S.C. § 119(e) to U.S. Provisional Application 62/835,295, filed on Apr. 17, 2019. The disclosure of this prior application is considered part of the disclosure of this application and is hereby incorporated by reference in its entirety.

### FIELD

The present disclosure relates to uses of computerized behavioral therapy in the treatment of depression (i.e., depressive symptoms) or depressive disorder associated with multiple sclerosis.

Use of computerized behavioral therapy in the treatment of:
depression (i.e., depressive symptoms) associated with multiple sclerosis;
depressive disorder associated with multiple sclerosis.

### BACKGROUND

Multiple sclerosis (MS) is a chronic, inflammatory demyelinating disorder of the central nervous system. Depression is common in patients with multiple sclerosis (MS), which substantially impairs their quality of life and it is associated with lower adherence to treatments for MS. Depression in patients with MS may affect body functions such as balance (Alghwiri, A. A., et al., in Multiple Sclerosis and Related Disorders, 24 (2018), 28-31), and it is associated with a higher rate of hospitalizations [Brenner, P., et al., in Acta Neurologica Scandinavica, 134 (Suppl. 200), 2016, 47-54]. Despite, depression is more prevalent in patients with MS than in the general population (i.e. about threefold that of the general population), no guidelines exist for the treatment of depression in MS. Moreover, reference to depression in the context of any neurological disease, such as MS, requires a distinction between depression as a depressive symptom (e.g. according to a rating scale, such as a self-report rating scale, for example, the Beck Depression Inventory) and depression as a formal diagnosis of a depressive disorder (e.g. according to diagnostic criteria consensus, such as the DSM-5).

Antidepressant medication, individual psychotherapy and group therapy are common treatments for depression in MS. Cognitive-behavioral therapy (CBT) has been shown beneficial in the treatment of depression in MS ((Feinstein, A., et al., in Nature Reviews 10 (2014), 507-517; Kidd T., et al., in PLOS ONE, 2017, 1-16) (Brenner, P., et al., in Acta Neurologica Scandinavica, 134 (Suppl. 200), 2016, 47-54)). In view of the prevalence and impact of depression in patients with MS and that the access to CBT can be difficult, based on cost and availability of psychotherapists, there is a need to develop computer-implemented CBT, that can help depressed patients with MS.

### BRIEF SUMMARY

The present disclosure relates to:

i) The use of a disease-modifying therapy of MS and a computer-implemented device in the treatment of depression associated with multiple sclerosis, wherein the computer-implemented device is an electronic device according to any one of Items A1-A7, A18 or Items B1-B8, B22-B25.
ii) The use of a disease-modifying therapy of MS and a digital-therapeutic in the treatment of depression associated with multiple sclerosis, wherein the digital-therapeutic (e.g., a digital therapeutic according to any one of Items A19-A26 or Items B26-B31) delivers behavioral therapy.
iii) The use of a disease-modifying therapy of MS in the treatment of depression associated with multiple sclerosis, wherein the use is combined with computer-implemented behavioral therapy (e.g., a method according to any one of Items A8-A17, A29-A31 or Items B9-B21).
i″) The use of an antidepressant and a computer-implemented device in the treatment of depression associated with multiple sclerosis, wherein the computer-implemented device is an electronic device according to any one of Items A1-A7, A18 or Items B1-B8, B22-B25.
ii″) The use of an antidepressant and a digital-therapeutic in the treatment of depression associated with multiple sclerosis, wherein the digital-therapeutic (e.g., a digital therapeutic according to any one of Items A19-A26 or Items B26-B31) delivers behavioral therapy.
iii″) The use of an antidepressant in the treatment of depression associated with multiple sclerosis, wherein the use is combined with computer-implemented behavioral therapy (e.g., a method according to any one of Items A8-A17, A29-A31 or Items B9-B21).

### BRIEF DESCRIPTION OF THE FIGURES

For a better understanding of the various described embodiments, reference should be made to the Description of Embodiments below, in conjunction with the following drawings, which are not necessarily drawn to scale, in which like reference numerals refer to corresponding parts throughout the figures.

FIG. 1 is a schematic view of an example system implementing a computerized method for treating depressive symptoms associated with multiple sclerosis.

FIG. 2A illustrates a feeling selection interface in accordance with an exemplary embodiment of the disclosure.

FIG. 2B illustrates a feeling spectrum interface in accordance with an exemplary embodiment of the disclosure.

FIG. 2C illustrates an automatic thought selection interface in accordance with an exemplary embodiment of the disclosure.

FIG. 2D illustrates an alternative thought selection interface in accordance with an exemplary embodiment of the disclosure.

FIG. 2E illustrates a feeling spectrum interface in accordance with an exemplary embodiment of the disclosure.

FIG. 2F illustrates a thinking traps interface in accordance with an exemplary embodiment of the disclosure.

FIG. 2G illustrates another view of the thinking traps interface in accordance with an exemplary embodiment of the disclosure.

FIG. 2H illustrates yet another view of the thinking traps interface in accordance with an exemplary embodiment of the disclosure.

FIG. 2I illustrates a company selection interface in accordance with an exemplary embodiment of the disclosure.

FIG. 2J illustrates a location selection interface in accordance with an exemplary embodiment of the disclosure.

FIG. 2K illustrates a symptoms selection interface in accordance with an exemplary embodiment of the disclosure.

FIG. 2L illustrates a recap interface element in accordance with an exemplary embodiment of the disclosure.

FIG. 2M illustrates a journal interface in accordance with an exemplary embodiment of the disclosure.

FIG. 2N illustrates a positive feeling selection interface in accordance with an exemplary embodiment of the disclosure.

FIG. 2O illustrates a situation selection interface in accordance with an exemplary embodiment of the disclosure.

FIG. 2P illustrates a positive reflection element in accordance with an exemplary embodiment of the disclosure.

FIG. 2Q illustrates a positive journal interface in accordance with an exemplary embodiment of the disclosure.

FIG. 2R illustrates a relax-and-remind interface in accordance with an exemplary embodiment of the disclosure.

FIG. 2S illustrates a mindfulness interface in accordance with an exemplary embodiment of the disclosure.

FIG. 2T illustrates a mindfulness technique data interface in accordance with an exemplary embodiment of the disclosure.

FIG. 2U illustrates a fatigue interface in accordance with an exemplary embodiment of the disclosure.

FIG. 2V illustrates a fatigue type data interface in accordance with an exemplary embodiment of the disclosure.

FIG. 3 is a flowchart illustrating a computerized method for treating depressive symptoms associated with multiple sclerosis in accordance with an exemplary embodiment of the disclosure.

FIG. 4 is a flowchart illustrating another computerized method for treating depressive symptoms associated with multiple sclerosis in accordance with an exemplary embodiment of the disclosure.

FIG. 5 is a schematic view of an example electronic device for treating depressive symptoms associated with multiple sclerosis in accordance with an exemplary embodiment of the disclosure.

FIG. 6 is a functional block diagram illustrating a digital therapeutic for treating depressive symptoms associated with multiple sclerosis in accordance with an exemplary embodiment of the disclosure.

Like reference symbols in the various drawings indicate like elements.

### DETAILED DESCRIPTION OF EMBODIMENTS

In one embodiment, it is found that a combination, as described herein, may be ideal for treating:
depression (i.e. depressive symptoms) associated with multiple sclerosis;
depressive disorder associated with multiple sclerosis;

having therapeutic advantages, such as one or more of the following:
i) it decreases, for example compared to a sham or placebo, severity of depression associated with multiple sclerosis, for example as assessed by the Montgomery-Åsberg Depression Rating Scale [MADRS; e.g. in Br. J. Psychiat. (1979), 134, 382-389] or the Beck Depression Inventory [e.g. BDI-II score; e.g. in Beck, A. T., Brown, G. K. (1996), Manual for the Beck Depression Inventory-II, San Antonio, Tex.: Psychological Corporation];
ii) it alleviates (e.g. by reducing), for example compared to a sham or placebo, depressive symptoms in patients with MS, for example as assessed from a self-report tool, for example, the Beck Depression Inventory (e.g. BDI-II);
iii) it increases retention of patients in treatment, for example, compared to a sham or placebo, for example it increases the rate of patient retention in a treatment program, such as a clinical setting (e.g. as measured by patient attendance at scheduled clinic visits, time to dropout from treatment or % dropout rate);
iv) it improves medication adherence, for example, it Improves adherence to disease-modifying therapy for MS, for example compared to a sham or placebo;
v) it improves cognitive function, for example compared to a sham or placebo, for example as assessed by a standard test;
vi) it decreases hospitalization rate, for example compared to a sham or placebo;
vii) it improves quality of life, for example as assessed by the MS Quality of Life Inventory (MSQLI), for example compared to a sham or placebo:
viii) it has a favorable therapeutic profile, for example compared to a sham or placebo, such as a favorable safety profile, for example a favorable safety profile in relation to adverse events.

Embodiments of the present disclosure include:

### Embodiments (a)-(j), Related to a Disease-Modifying Therapy

### Embodiments (a)

1a. A combination for use in the treatment of depression associated with multiple sclerosis comprising a disease-modifying therapy for multiple sclerosis and a computer-implemented device, wherein the computer-implemented device is an electronic device according to any one of Items A1-A7, A18 or Items B1-B8, B22-B25.
2a. A combination for use in reducing depressive symptoms associated with multiple sclerosis comprising a disease-modifying therapy for multiple sclerosis and a computer-implemented device, wherein the computer-implemented device is an electronic device according to any one of Items A1-A7, A18 or Items B1-B8, B22-B25.
3a. The combination for use according to embodiment 2a, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4a. The combination for use according to embodiment 1a, wherein depression associated with multiple sclerosis is selected from the group consisting of mild depression associated with multiple sclerosis, moderate depression associated with multiple sclerosis or severe depression associated with multiple sclerosis.
5a. The combination for use according to embodiment 4a, wherein mild depression, moderate depression and severe depression are characterized according to the total score of depressive symptom severity of the Montgomery-Åsberg Depression Rating Scale or of the Beck Depression Inventory.
6a. A combination for use in the treatment of depressive disorder associated with multiple sclerosis comprising a disease-modifying therapy for multiple sclerosis and a computer-implemented device, wherein the computer-implemented device is an electronic device according to any one of Items A1-A7, A18 or Items B1-B8, B22-B25.
7a. The combination for use according to embodiment 6a, wherein the depressive disorder associated with multiple sclerosis is depressive disorder due to a medical condition or major depressive disorder, in particular major depressive disorder.
8a. The combination for use according to any one of embodiments 1a to 7a, wherein the computer-implemented device is a computer-implemented mobile device, for example selected from the group consisting of a smartphone, a laptop computer, a tablet computer and a wearable computer; in particular a smartphone.
9a. The combination for use according to any one of embodiments 1a to 8a, wherein the computer-implemented device is system-prompted or on-demand.
10a. The combination for use according to any one of embodiments 1a to 9a, which comprises one or more further active agent (i.e. at least one further active agent).
11a. The combination for use according to embodiment 10a, wherein the disease-modifying therapy for multiple sclerosis and the one or more active agent (i.e. at least one further active agent) is in the form of a fixed pharmaceutical combination or a non-fixed pharmaceutical combination.
12a. The combination for use according to embodiments 10a or 11a, wherein the further active agent is selected from the group consisting of an antidepressant and an anxiolytic.
13a. The combination for use according to any one of embodiments 1a to 12a, wherein the computer-implemented device delivers behavioral therapy, such as cognitive behavioral therapy.
14a. The combination for use according to embodiment 13a, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
15a. The combination for use according to embodiment 13a, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
16a. The combination for use according to any one of embodiments 1a to 12a, wherein the behavioral therapy is cognitive behavioral therapy for depressive disorder associated with multiple sclerosis.
17a. The combination for use according to embodiment 16a, wherein the cognitive behavioral therapy for depressive disorder associated with multiple sclerosis focuses on mood management and coping with symptoms of multiple sclerosis.
18a. The combination for use according to embodiment 16a, wherein the cognitive behavioral therapy for depressive disorder associated with multiple sclerosis focuses on mood management or coping with symptoms of multiple sclerosis.
19a. The combination for use according to any one of embodiments 1a to 18a, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.
20a. A combination comprising a disease-modifying therapy for multiple sclerosis and a computer-implemented device, wherein the computer-implemented device is an electronic device according to any one of Items A1-A7, A18 or Items B1-B8, B22-B25.
21a. The combination according to embodiment 20a, which comprises one or more further active agent (i.e. at least one further active agent).
22a. The combination according to embodiment 21a, wherein the disease-modifying therapy for multiple sclerosis and the one or more active agent (i.e. at least one further active agent) is in the form of a fixed pharmaceutical combination or a non-fixed pharmaceutical combination.
23a. The combination according to embodiments 21a or 22a, wherein the further active agent is selected from the group consisting of an antidepressant and an anxiolytic.
24a. The combination according to embodiments 20a to 23a, wherein the computer-implemented device is a computer-implemented mobile device, for example selected from the group consisting of a smartphone, a laptop computer, a tablet computer and a wearable computer; in particular a smartphone.

### Embodiments (b)

1b. A combination for use in the treatment of depression associated with multiple sclerosis comprising a disease-modifying therapy for multiple sclerosis and a digital-therapeutic, wherein the digital-therapeutic (e.g., a digital therapeutic according to any one of Items A17-A24 or Items B26-B31) delivers behavioral therapy, such as cognitive behavioral therapy.
2b. A combination for use in reducing depressive symptoms associated with multiple sclerosis comprising a disease-modifying therapy for multiple sclerosis and a digital-therapeutic, wherein the digital-therapeutic (e.g., a digital therapeutic according to any one of Items A17-A24 or Items B26-B31) delivers behavioral therapy, such as cognitive behavioral therapy.
3b. The combination for use according to embodiment 2b, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4b. The combination for use according to embodiment 1b, wherein depression associated with multiple sclerosis is selected from the group consisting of mild depression associated with multiple sclerosis, moderate depression associated with multiple sclerosis or severe depression associated with multiple sclerosis.
5b. The combination for use according to embodiment 4b, wherein mild depression, moderate depression and severe depression are characterized according to the total score of depressive symptom severity of the Montgomery-Åsberg Depression Rating Scale or of the Beck Depression Inventory.
6b. A combination for use in the treatment of depressive disorder associated with multiple sclerosis comprising a disease-modifying therapy for multiple sclerosis and a digital-therapeutic, wherein the digital-therapeutic (e.g., a digital therapeutic according to any one of Items A17-A24 or Items B26-B31) delivers behavioral therapy, such as cognitive behavioral therapy.
7b. The combination for use according to embodiment 6b, wherein the depressive disorder associated with multiple sclerosis is depressive disorder due to a medical condition or major depressive disorder, in particular major depressive disorder.
8b. The combination for use according to any one of embodiments 1b to 7b, wherein the digital-therapeutic is system-prompted or on-demand.
9b. The combination for use according to any one of embodiments 1b to 8b, wherein the digital-therapeutic comprises a mobile device, for example selected from the group consisting of a smartphone, a laptop computer, a tablet computer and a wearable computer; in particular a smartphone.

10b. The combination for use according to any one of embodiments 1b to 9b, which comprises one or more further active agent (i.e. at least one further active agent).

11b. The combination for use according to embodiment 10b, wherein the disease-modifying therapy for multiple sclerosis and the one or more active agent (i.e. at least one further active agent) is in the form of a fixed pharmaceutical combination or a non-fixed pharmaceutical combination.
12b. The combination for use according to embodiments 10b or 11b, wherein the further active agent is selected from the group consisting of an antidepressant and an anxiolytic.
13b. The combination for use according to any one of embodiments 1b to 12b, wherein the behavioral therapy is cognitive behavioral therapy for depression associated with multiple sclerosis.
14b. The combination for use according to embodiment 13b, wherein the cognitive behavioral therapy for depression associated with multiple sclerosis focuses on mood management and coping with symptoms of multiple sclerosis.
15b. The combination for use according to embodiment 13b, wherein the cognitive behavioral therapy for depression associated with multiple sclerosis focuses on mood management or coping with symptoms of multiple sclerosis.
16b. The combination for use according to any one of embodiments 1b to 12b, wherein the behavioral therapy is cognitive behavioral therapy for depressive disorder associated with multiple sclerosis.
17b. The combination for use according to embodiment 16b, wherein the cognitive behavioral therapy for depressive disorder associated with multiple sclerosis focuses on mood management and coping with symptoms of multiple sclerosis.
18b. The combination for use according to embodiment 16b, wherein the cognitive behavioral therapy for depressive disorder associated with multiple sclerosis focuses on mood management or coping with symptoms of multiple sclerosis.
19b. The combination for use according to any one of embodiments 1b to 18b, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (c)

1c. A disease-modifying therapy for multiple sclerosis for use in the treatment of depression associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
2c. A disease-modifying therapy for multiple sclerosis for use in reducing depressive symptoms associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
3c. The disease-modifying therapy for multiple sclerosis for use according to embodiment 2c, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4c. The disease-modifying therapy for multiple sclerosis for use according to embodiment 1c, wherein depression associated with multiple sclerosis is selected from the group consisting of mild depression associated with multiple sclerosis, moderate depression associated with multiple sclerosis or severe depression associated with multiple sclerosis.
5c. The disease-modifying therapy for multiple sclerosis for use according to embodiment 4c, wherein mild depression, moderate depression and severe depression are characterized according to the total score of depressive symptom severity of the Montgomery-Åsberg Depression Rating Scale or of the Beck Depression Inventory.
6c. A disease-modifying therapy for multiple sclerosis for use in the treatment of depressive disorder associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
7c. The disease-modifying therapy for multiple sclerosis for use according to embodiment 6c, wherein the depressive disorder associated with multiple sclerosis is depressive disorder due to a medical condition or major depressive disorder, in particular major depressive disorder.
8c. The disease-modifying therapy for multiple sclerosis for use according to any one of embodiments 1c to 7c, wherein the computer-implemented behavioral therapy, such as computer-implemented cognitive behavioral therapy, is system-prompted or on-demand.
9c. The disease-modifying therapy for multiple sclerosis for use according to any one of embodiments 1c to 8c, wherein the computer-implemented behavioral therapy is provided by a computerized device.
10c. The disease-modifying therapy for multiple sclerosis for use according to embodiment 9c, wherein the computerized device is a mobile device.
11c. The disease-modifying therapy for multiple sclerosis for use according to embodiment 10c, wherein the mobile device is selected from the group consisting of a smartphone, a laptop computer, a tablet computer, and a wearable computer; in particular a smartphone.
12c. The disease-modifying therapy for multiple sclerosis for use according to any one of embodiments 1c to 11c, wherein the disease-modifying therapy is in the form of a pharmaceutical combination comprising one or more further active agent (i.e. at least one further active agent).
13c. The disease-modifying therapy for multiple sclerosis for use according to embodiment 12c, wherein the pharmaceutical combination is a fixed pharmaceutical combination or a non-fixed pharmaceutical combination.
14c. The disease-modifying therapy for multiple sclerosis for use according to embodiments 12c or 13c, wherein the one or more active agent (i.e. at least one further active agent) is selected from the group consisting of an antidepressant and an anxiolytic.
15c. The disease-modifying therapy for multiple sclerosis for use according to any one of embodiments 1c to 14c, wherein the behavioral therapy is cognitive behavioral therapy for depression associated with multiple sclerosis.
16c. The disease-modifying therapy for multiple sclerosis for use according to embodiment 15c, wherein the cognitive behavioral therapy for depression associated with multiple sclerosis focuses on mood management and coping with symptoms of multiple sclerosis.
17c. The disease-modifying therapy for multiple sclerosis for use according to embodiment 15c, wherein the cognitive behavioral therapy for depression associated with multiple sclerosis focuses on mood management or coping with symptoms of multiple sclerosis.
18c. The disease-modifying therapy for multiple sclerosis for use according to any one of embodiments 1c to 14c, wherein the behavioral therapy is cognitive behavioral therapy for depressive disorder associated with multiple sclerosis.
19c. The disease-modifying therapy for multiple sclerosis for use according to embodiment 18c, wherein the cognitive behavioral therapy for depressive disorder associated with multiple sclerosis focuses on mood management and coping with symptoms of multiple sclerosis.
20c. The disease-modifying therapy for multiple sclerosis for use according to embodiment 18c, wherein the cognitive behavioral therapy for depressive disorder associated with multiple sclerosis focuses on mood management or coping with symptoms of multiple sclerosis.
21c. The disease-modifying therapy for multiple sclerosis for use according to any one of embodiments 1c to 20c, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (d)

1d. Use of a disease-modifying therapy for multiple sclerosis for the manufacture of a medicament for the treatment of depression associated with multiple sclerosis, wherein
i) the medicament is provided in combination with a computer-implemented device; and
ii) the computer-implemented device is an electronic device according to any one of Items A1-A7, A18 or Items B1-B8, B22-B25.
   2d. Use of a disease-modifying therapy for multiple sclerosis for the manufacture of a medicament for a treatment to reduce depressive symptoms associated with multiple sclerosis, wherein
i) the medicament is provided in combination with a computer-implemented device; and
ii) the computer-implemented device is an electronic device according to any one of Items A1-A7, A18 or Items B1-B8, B22-B25.
   3d. The use according to embodiment 2d, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4d. The use according to embodiment 1d, wherein depression associated with multiple sclerosis is selected from the group consisting of mild depression associated with multiple sclerosis, moderate depression associated with multiple sclerosis or severe depression associated with multiple sclerosis.
   5d. The use according to embodiment 4d, wherein mild depression, moderate depression and severe depression are characterized according to the total score of depressive symptom severity of the Montgomery-Åsberg Depression Rating Scale or of the Beck Depression Inventory.
   6d. Use of a disease-modifying therapy for multiple sclerosis for the manufacture of a medicament for the treatment of depressive disorder associated with multiple sclerosis, wherein
i) the medicament is provided in combination with a computer-implemented device; and
ii) the computer-implemented device is an electronic device according to any one of Items A1-A7, A18 or Items B1-B8, B22-B25.
   7d. The use according to embodiment 6d, wherein the depressive disorder associated with multiple sclerosis is depressive disorder due to a medical condition or major depressive disorder, in particular major depressive disorder.
   8d. The use according to any one of embodiments 1d to 7d, wherein the computer-implemented behavioral therapy, such as computer-implemented cognitive behavioral therapy, is system-prompted or on-demand.
   9d. The use according to any one of embodiments 1d to 8d, wherein the computer-implemented device is a computer-implemented mobile device, for example selected from the group consisting of a smartphone, a laptop computer, a tablet computer and a wearable computer; in particular a smartphone.
   10d. The use according to any one of embodiments 1d to 9d, which comprises one or more further active agent (i.e. at least one further active agent).
   11d. The use according to embodiment 10d, wherein the disease-modifying therapy for multiple sclerosis and the one or more active agent (i.e. at least one further active agent) is in the form of a fixed pharmaceutical combination or a non-fixed pharmaceutical combination.
   12d. The use according to embodiments 10d or 11d, wherein the further active agent is selected from the group consisting of an antidepressant and an anxiolytic.
   13d. The use according to any one of embodiments 1d to 11d, wherein the computer-implemented device delivers behavioral therapy, such as cognitive behavioral therapy.
   14d. The use according to embodiment 13d, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
   15d. The use according to embodiment 13d, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
   16d. The use according to any one of embodiments 1d to 12d, wherein the behavioral therapy is cognitive behavioral therapy for depressive disorder associated with multiple sclerosis.
   17d. The use according to embodiment 16d, wherein the cognitive behavioral therapy for depressive disorder associated with multiple sclerosis focuses on mood management and coping with symptoms of multiple sclerosis.
   18d. The use according to embodiment 16d, wherein the cognitive behavioral therapy for depressive disorder associated with multiple sclerosis focuses on mood management or coping with symptoms of multiple sclerosis.
   19d. The use according to any one of embodiments 1d to 18d, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (e)

1e. Use of a disease-modifying therapy for multiple sclerosis for the manufacture of a medicament for the treatment of depression associated with multiple sclerosis, wherein
i) the medicament is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   2e. Use of a disease-modifying therapy for multiple sclerosis for the manufacture of a medicament for a treatment to reduce depressive symptoms associated with multiple sclerosis, wherein
i) the medicament is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   3e. The use according to embodiment 2e, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4e. The use according to embodiment 1e, wherein depression associated with multiple sclerosis is selected from the group consisting of mild depression associated with multiple sclerosis, moderate depression associated with multiple sclerosis or severe depression associated with multiple sclerosis.
   5e. The use according to embodiment 4e, wherein mild depression, moderate depression and severe depression are characterized according to the total score of depressive symptom severity of the Montgomery-Åsberg Depression Rating Scale or of the Beck Depression Inventory.
   6e. Use of a disease-modifying therapy for multiple sclerosis for the manufacture of a medicament for the treatment of depressive disorder associated with multiple sclerosis, wherein
i) the medicament is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   7e. The use according to embodiment 6e, wherein the depressive disorder associated with multiple sclerosis is depressive disorder due to a medical condition or major depressive disorder, in particular major depressive disorder.
   8e. The use according to any one of embodiments 1e to 7e, wherein the digital-therapeutic is system-prompted or on-demand.
   9e. The use according to any one of embodiments 1e to 8e, wherein the digital-therapeutic comprises a mobile device, for example selected from the group consisting of a smartphone, a laptop computer, a tablet computer, and a wearable computer; in particular a smartphone.
   10e. The use according to any one of embodiments 1e to 9e, wherein the disease-modifying therapy for multiple sclerosis is in the form of a pharmaceutical combination comprising one or more further active agent (i.e. at least one further active agent).
   11e. The use according to embodiment 10e, wherein the pharmaceutical combination is a fixed pharmaceutical combination or a non-fixed pharmaceutical combination.
   12e. The use according to embodiments 10e or 11e, wherein the one or more active agent (i.e. at least one further active agent) is selected from the group consisting of an antidepressant and an anxiolytic.
   13e. The use according to any one of embodiments 1e to 12e, wherein the behavioral therapy is cognitive behavioral therapy for depression associated with multiple sclerosis.
   14e. The use according to embodiment 13e, wherein the cognitive behavioral therapy for depression associated with multiple sclerosis focuses on mood management and coping with symptoms of multiple sclerosis.
   15e. The use according to embodiment 13e, wherein the cognitive behavioral therapy for depression associated with multiple sclerosis focuses on mood management or coping with symptoms of multiple sclerosis.
   16e. The use according to any one of embodiments 1e to 12e, wherein the behavioral therapy is cognitive behavioral therapy for depressive disorder associated with multiple sclerosis.
   17e. The use according to embodiment 16e, wherein the cognitive behavioral therapy for depressive disorder associated with multiple sclerosis focuses on mood management and coping with symptoms of multiple sclerosis.
   18e. The use according to embodiment 16e, wherein the cognitive behavioral therapy for depressive disorder associated with multiple sclerosis focuses on mood management or coping with symptoms of multiple sclerosis.
   19e. The use according to any one of embodiments 1e to 18e, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (f)

1f. Use of a disease-modifying therapy for multiple sclerosis for the manufacture of a medicament for the treatment of depression associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
2f. Use of a disease-modifying therapy for multiple sclerosis for the manufacture of a medicament for a treatment to reduce depressive symptoms associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
3f. The use according to embodiment 2f, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4f. The use according to embodiment 1f, wherein depression associated with multiple sclerosis is selected from the group consisting of mild depression associated with multiple sclerosis, moderate depression associated with multiple sclerosis or severe depression associated with multiple sclerosis.
5f. The use according to embodiment 4f, wherein mild depression, moderate depression and severe depression are characterized according to the total score of depressive symptom severity of the Montgomery-Åsberg Depression Rating Scale or of the Beck Depression Inventory.
6f. Use of a disease-modifying therapy for multiple sclerosis for the manufacture of a medicament for the treatment of depressive disorder associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
7f. The use according to embodiment 6f, wherein the depressive disorder associated with multiple sclerosis is depressive disorder due to a medical condition or major depressive disorder, in particular major depressive disorder.
8f. The use according to any one of embodiments 1f to 7f, wherein the computer-implemented behavioral therapy, such as computer-implemented cognitive behavioral therapy, is system-prompted or on-demand.
9f. The use according to any one of embodiments 1f to 8f, wherein the computer-implemented behavioral therapy is provided by a computerized device.
10f. The use according to embodiment 9f, wherein the computerized device is a mobile device.
11f. The use according to embodiment 10f, wherein the mobile device is selected from the group consisting of a smartphone, a laptop computer, a tablet computer, and a wearable computer; in particular a smartphone.
12f. The use according to any one of embodiments 1f to 11f, wherein the disease-modifying therapy for multiple sclerosis is in the form of a pharmaceutical combination comprising one or more further active agent (i.e. at least one further active agent).
13f. The use according to embodiment 12f, wherein the pharmaceutical combination is a fixed pharmaceutical combination or a non-fixed pharmaceutical combination.
14f. The use according to embodiments 12f or 13f, wherein the one or more active agent (i.e. at least one further active agent) is selected from the group consisting of an antidepressant and an anxiolytic.
15f. The use according to any one of embodiments 1f to 14f, wherein the behavioral therapy is cognitive behavioral therapy for depression associated with multiple sclerosis.
16f. The use according to embodiment 15f, wherein the cognitive behavioral therapy for depression associated with multiple sclerosis focuses on mood management and coping with symptoms of multiple sclerosis.
17f. The use according to embodiment 15f, wherein the cognitive behavioral therapy for depression associated with multiple sclerosis focuses on mood management or coping with symptoms of multiple sclerosis.
18f. The use according to any one of embodiments 1f to 14f, wherein the behavioral therapy is cognitive behavioral therapy for depressive disorder associated with multiple sclerosis.
19f. The use according to embodiment 18f, wherein the cognitive behavioral therapy for depressive disorder associated with multiple sclerosis focuses on mood management and coping with symptoms of multiple sclerosis.
20f. The use according to embodiment 18f, wherein the cognitive behavioral therapy for depressive disorder associated with multiple sclerosis focuses on mood management or coping with symptoms of multiple sclerosis.
21f. The use according to any one of embodiments 1f to 20f, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (g)

1g. A method for the treatment of depression associated with multiple sclerosis, in a subject, in need thereof, comprising administering to said subject an effective amount of a disease-modifying therapy for multiple sclerosis, wherein
i) the disease-modifying therapy for multiple sclerosis is provided in combination with a computer-implemented device; and
ii) the computer-implemented device is an electronic device according to any one of Items A1-A7, A18 or Items B1-B8, B22-B25.
   2g. A method for a treatment to reduce depressive symptoms associated with multiple sclerosis, in a subject, in need thereof, comprising administering to said subject an effective amount of a disease-modifying therapy for multiple sclerosis, wherein
i) the disease-modifying therapy for multiple sclerosis is provided in combination with a computer-implemented device; and
ii) the computer-implemented device is an electronic device according to any one of Items A1-A7, A18 or Items B1-B8, B22-B25.
   3g. The method according to embodiment 2g, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4g. The method according to embodiment 1g, wherein depression associated with multiple sclerosis is selected from the group consisting of mild depression associated with multiple sclerosis, moderate depression associated with multiple sclerosis or severe depression associated with multiple sclerosis.
   5g. The method according to embodiment 4g, wherein mild depression, moderate depression and severe depression are characterized according to the total score of depressive symptom severity of the Montgomery-Åsberg Depression Rating Scale or of the Beck Depression Inventory.
   6g. A method for the treatment of depressive disorder associated with multiple sclerosis, in a subject, in need thereof, comprising administering to said subject an effective amount of a disease-modifying therapy for multiple sclerosis, wherein
i) the disease-modifying therapy for multiple sclerosis is provided in combination with a computer-implemented device; and
ii) the computer-implemented device is an electronic device according to any one of Items A1-A7, A18 or Items B1-B8, B22-B25.
   7g. The method according to embodiment 6g, wherein the depressive disorder associated with multiple sclerosis is depressive disorder due to a medical condition or major depressive disorder, in particular major depressive disorder.
   8g. The method according to any one of embodiments 1g to 7g, wherein the computer-implemented behavioral therapy, such as computer-implemented cognitive behavioral therapy, is system-prompted or on-demand.
   9g. The method according to any one of embodiments 1g to 8g, wherein the computer-implemented device is a computer-implemented mobile device, for example selected from the group consisting of a smartphone, a laptop computer, a tablet computer and a wearable computer; in particular a smartphone.
   10g. The method according to any one of embodiments 1g to 9g, which comprises one or more further active agent (i.e. at least one further active agent).
   11g. The method according to embodiment 10g, wherein the disease-modifying therapy for multiple sclerosis and the one or more active agent (i.e. at least one further active agent) is in the form of a fixed pharmaceutical combination or a non-fixed pharmaceutical combination.
   12g. The method according to embodiments 10g or 11g, wherein the further active agent is selected from the group consisting of an antidepressant and an anxiolytic.
   13g. The method according to any one of embodiments 1g to 11g, wherein the computer-implemented device delivers behavioral therapy, such as cognitive behavioral therapy.
   14g. The method according to embodiment 13g, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
   15g. The method according to embodiment 13g, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
   16g. The method according to any one of embodiments 1g to 12g, wherein the behavioral therapy is cognitive behavioral therapy for depressive disorder associated with multiple sclerosis.
   17g. The method according to embodiment 16g, wherein the cognitive behavioral therapy for depressive disorder associated with multiple sclerosis focuses on mood management and coping with symptoms of multiple sclerosis.
   18g. The method according to embodiment 16g, wherein the cognitive behavioral therapy for depressive disorder associated with multiple sclerosis focuses on mood management or coping with symptoms of multiple sclerosis.
   19g. The method according to any one of embodiments 1g to 18g, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (h)

1h. A method for the treatment of depression associated with multiple sclerosis, in a subject, in need thereof, comprising administering to said subject an effective amount of a disease-modifying therapy for multiple sclerosis, wherein
i) the disease-modifying therapy for multiple sclerosis is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   2h. A method for a treatment to reduce depressive symptoms associated with multiple sclerosis, in a subject, in need thereof, comprising administering to said subject an effective amount of a disease-modifying therapy for multiple sclerosis, wherein
i) the disease-modifying therapy for multiple sclerosis is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   3h. The method according to embodiment 2h, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4h. The method according to embodiment 1h, wherein depression associated with multiple sclerosis is selected from the group consisting of mild depression associated with multiple sclerosis, moderate depression associated with multiple sclerosis or severe depression associated with multiple sclerosis.
   5h. The method according to embodiment 4h, wherein mild depression, moderate depression and severe depression are characterized according to the total score of depressive symptom severity of the Montgomery-Åsberg Depression Rating Scale or of the Beck Depression Inventory.
   6h. A method for the treatment of depressive disorder associated with multiple sclerosis, in a subject, in need thereof, comprising administering to said subject an effective amount of a disease-modifying therapy for multiple sclerosis, wherein
i) the disease-modifying therapy for multiple sclerosis is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   7h. The method according to embodiment 6h, wherein the depressive disorder associated with multiple sclerosis is depressive disorder due to a medical condition or major depressive disorder, in particular major depressive disorder.
   8h. The method according to any one of embodiments 1h to 7h, wherein the digital-therapeutic is system-prompted or on-demand.
   9h. The method according to any one of embodiments 1h to 8h, wherein the digital-therapeutic comprises a mobile device, for example selected from the group consisting of a smartphone, a laptop computer, a tablet computer, and a wearable computer; in particular a smartphone.
   10h. The method according to any one of embodiments 1h to 9h, wherein the disease-modifying therapy for multiple sclerosis is in the form of a pharmaceutical combination comprising one or more further active agent (i.e. at least one further active agent).
   11h. The method according to embodiment 10h, wherein the pharmaceutical combination is a fixed pharmaceutical combination or a non-fixed pharmaceutical combination.
   12h. The method according to embodiments 10h or 11h, wherein the one or more active agent (i.e. at least one further active agent) is selected from the group consisting of an antidepressant and an anxiolytic.
   13h. The method according to any one of embodiments 1h to 12h, wherein the behavioral therapy is cognitive behavioral therapy for depression associated with multiple sclerosis.
   14h. The method according to embodiment 13h, wherein the cognitive behavioral therapy for depression associated with multiple sclerosis focuses on mood management and coping with symptoms of multiple sclerosis.
   15h. The method according to embodiment 13h, wherein the cognitive behavioral therapy for depression associated with multiple sclerosis focuses on mood management or coping with symptoms of multiple sclerosis.
   16h. The method according to any one of embodiments 1h to 12h, wherein the behavioral therapy is cognitive behavioral therapy for depressive disorder associated with multiple sclerosis.
   17h. The method according to embodiment 16h, wherein the cognitive behavioral therapy for depressive disorder associated with multiple sclerosis focuses on mood management and coping with symptoms of multiple sclerosis.
   18h. The method according to embodiment 16h, wherein the cognitive behavioral therapy for depressive disorder associated with multiple sclerosis focuses on mood management or coping with symptoms of multiple sclerosis.
   19h. The method according to any one of embodiments 1h to 18h, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (j)

1j. A method for the treatment of depression associated with multiple sclerosis, in a subject, in need thereof, comprising administering to said subject an effective amount of a disease-modifying therapy for multiple sclerosis, wherein the method is combined with computer-implemented behavioral therapy (e.g., a method according to any one of Items A8-A17, A29-A31 or Items B9-B21), such as computer-implemented cognitive behavioral therapy.
2j. A method for a treatment to reduce depressive symptoms associated with multiple sclerosis, in a subject, in need thereof, comprising administering to said subject an effective amount of a disease-modifying therapy for multiple sclerosis, wherein the method is combined with computer-implemented behavioral therapy (e.g., a method according to any one of Items A8-A17, A29-A31 or Items B9-B21), such as computer-implemented cognitive behavioral therapy.
3j. The method according to embodiment 2j, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4j. The method according to embodiment 1j, wherein depression associated with multiple sclerosis is selected from the group consisting of mild depression associated with multiple sclerosis, moderate depression associated with multiple sclerosis or severe depression associated with multiple sclerosis.
5j. The method according to embodiment 4j, wherein mild depression, moderate depression and severe depression are characterized according to the total score of depressive symptom severity of the Montgomery-Åsberg Depression Rating Scale or of the Beck Depression Inventory.
6j. A method for the treatment of depressive disorder associated with multiple sclerosis, in a subject, in need thereof, comprising administering to said subject an effective amount of a disease-modifying therapy for multiple sclerosis, wherein the method is combined with computer-implemented behavioral therapy (e.g., a method according to any one of Items A8-A17, A29-A31 or Items B9-B21), such as computer-implemented cognitive behavioral therapy.
7j. The method according to embodiment 6j, wherein the depressive disorder associated with multiple sclerosis is depressive disorder due to a medical condition or major depressive disorder, in particular major depressive disorder.
8j. The method according to any one of embodiments 1j to 7j, wherein the computer-implemented behavioral therapy, such as computer-implemented cognitive behavioral therapy, is system-prompted or on-demand.
9j. The method according to any one of embodiments 1j to 8j, wherein the computer-implemented behavioral therapy is provided by a computerized device.
10j. The method according to embodiment 9j, wherein the computerized device is a mobile device.
11j. The method according to embodiment 10j, wherein the mobile device is selected from the group consisting of a smartphone, a laptop computer, a tablet computer, and a wearable computer; in particular a smartphone.
12j. The method according to any one of embodiments 1j to 11j, wherein the disease-modifying therapy for multiple sclerosis is in the form of a pharmaceutical combination comprising one or more further active agent (i.e. at least one further active agent).
13j. The method according to embodiment 12j, wherein the pharmaceutical combination is a fixed pharmaceutical combination or a non-fixed pharmaceutical combination.
14j. The method according to embodiments 12j or 13j, wherein the one or more active agent (i.e. at least one further active agent) is selected from the group consisting of an antidepressant and an anxiolytic.
15j. The method according to any one of embodiments 1j to 14j, wherein the behavioral therapy is cognitive behavioral therapy for depression associated with multiple sclerosis.
16j. The method according to embodiment 15j, wherein the cognitive behavioral therapy for depression associated with multiple sclerosis focuses on mood management and coping with symptoms of multiple sclerosis.
17j. The method according to embodiment 15j, wherein the cognitive behavioral therapy for depression associated with multiple sclerosis focuses on mood management or coping with symptoms of multiple sclerosis.
18j. The method according to any one of embodiments 1j to 14j, wherein the behavioral therapy is cognitive behavioral therapy for depressive disorder associated with multiple sclerosis.
19j. The method according to embodiment 18j, wherein the cognitive behavioral therapy for depressive disorder associated with multiple sclerosis focuses on mood management and coping with symptoms of multiple sclerosis.
20j. The method according to embodiment 18j, wherein the cognitive behavioral therapy for depressive disorder associated with multiple sclerosis focuses on mood management or coping with symptoms of multiple sclerosis.
21j. The method according to any one of embodiments 1j to 20j, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (a″)-(j″), Related to an Antidepressant

### Embodiments (a″)

1a″. A combination for use in the treatment of depression associated with multiple sclerosis comprising an antidepressant and a computer-implemented device, wherein the computer-implemented device is an electronic device according to any one of Items A1-A7, A18 or Items B1-B8, B22-B25.

2a″. A combination for use in reducing depressive symptoms associated with multiple sclerosis comprising an antidepressant and a computer-implemented device, wherein the computer-implemented device is an electronic device according to any one of Items A1-A7, A18 or Items B1-B8, B22-B25.
3a″. The combination for use according to embodiment 2a″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4a″. The combination for use according to embodiment 1a″, wherein depression associated with multiple sclerosis is selected from the group consisting of mild depression associated with multiple sclerosis, moderate depression associated with multiple sclerosis or severe depression associated with multiple sclerosis.
5a″. The combination for use according to embodiment 4a″, wherein mild depression, moderate depression and severe depression are characterized according to the total score of depressive symptom severity of the Montgomery-Åsberg Depression Rating Scale or of the Beck Depression Inventory.
6a″. A combination for use in the treatment of depressive disorder associated with multiple sclerosis comprising an antidepressant and a computer-implemented device, wherein the computer-implemented device is an electronic device according to any one of Items A1-A7, A18 or Items B1-B8, B22-B25.
7a″. The combination for use according to embodiment 6a″, wherein the depressive disorder associated with multiple sclerosis is depressive disorder due to a medical condition or major depressive disorder, in particular major depressive disorder.
8a″. The combination for use according to any one of embodiments 1a″ to 7a″, wherein the computer-implemented device is a computer-implemented mobile device, for example selected from the group consisting of a smartphone, a laptop computer, a tablet computer and a wearable computer; in particular a smartphone.
9a″. The combination for use according to any one of embodiments 1a″ to 8a″, wherein the computer-implemented device is system-prompted or on-demand.
10a″. The combination for use according to any one of embodiments 1a″ to 9a″, which comprises one or more further active agent (i.e. at least one further active agent).
11a″. The combination for use according to embodiment 10a″, wherein the antidepressant and the one or more active agent (i.e. at least one further active agent) is in the form of a fixed pharmaceutical combination or a non-fixed pharmaceutical combination.
12a″. The combination for use according to embodiments 10a″ or 11a″, wherein the further active agent is selected from the group consisting of a disease-modifying therapy for multiple sclerosis and an anxiolytic.
13a″. The combination for use according to any one of embodiments 1a″ to 11a″, wherein the computer-implemented device delivers behavioral therapy, such as cognitive behavioral therapy.
14a″. The combination for use according to embodiment 13a″, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
15a″. The combination for use according to embodiment 13a″, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
16a″. The combination for use according to any one of embodiments 1a″ to 12a″, wherein the behavioral therapy is cognitive behavioral therapy for depressive disorder associated with multiple sclerosis.
17a″. The combination for use according to embodiment 16a″, wherein the cognitive behavioral therapy for depressive disorder associated with multiple sclerosis focuses on mood management and coping with symptoms of multiple sclerosis.
18a″. The combination for use according to embodiment 16a″, wherein the cognitive behavioral therapy for depressive disorder associated with multiple sclerosis focuses on mood management or coping with symptoms of multiple sclerosis.
19a″. The combination for use according to any one of embodiments 1a″ to 18a″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.
20a″. A combination comprising an antidepressant and a computer-implemented device, wherein the computer-implemented device is an electronic device according to any one of Items A1-A7, A18 or Items B1-B8, B22-B25.
21a″. The combination according to embodiment 20a″, which comprises one or more further active agent (i.e. at least one further active agent).
22a″. The combination according to embodiment 21a″, wherein the antidepressant and the one or more active agent (i.e. at least one further active agent) is in the form of a fixed pharmaceutical combination or a non-fixed pharmaceutical combination.
23a″. The combination according to embodiments 21a″ or 22a″, wherein the further active agent is selected from the group consisting of a disease-modifying therapy for multiple sclerosis and an anxiolytic.
24a″. The combination according to any one of embodiments 20a″ to 23a″, wherein the computer-implemented device is a computer-implemented mobile device, for example selected from the group consisting of a smartphone, a laptop computer, a tablet computer and a wearable computer; in particular a smartphone.

### Embodiments (b″)

1b″. A combination for use in the treatment of depression associated with multiple sclerosis comprising an antidepressant and a digital-therapeutic, wherein the digital-therapeutic (e.g., a digital therapeutic according to any one of Items A19-A26 or Items B26-B31) delivers behavioral therapy, such as cognitive behavioral therapy.
2b″. A combination for use in reducing depressive symptoms associated with multiple sclerosis comprising an antidepressant and a digital-therapeutic, wherein the digital-therapeutic (e.g., a digital therapeutic according to any one of Items A19-A26 or Items B26-B31) delivers behavioral therapy, such as cognitive behavioral therapy.
3b″. The combination for use according to embodiment 2b″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4b″. The combination for use according to embodiment 1b″, wherein depression associated with multiple sclerosis is selected from the group consisting of mild depression associated with multiple sclerosis, moderate depression associated with multiple sclerosis or severe depression associated with multiple sclerosis.
5b″. The combination for use according to embodiment 4b″, wherein mild depression, moderate depression and severe depression are characterized according to the total score of depressive symptom severity of the Montgomery-Åsberg Depression Rating Scale or of the Beck Depression Inventory.
6b″. A combination for use in the treatment of depressive disorder associated with multiple sclerosis comprising an antidepressant and a digital-therapeutic, wherein the digital-therapeutic (e.g., a digital therapeutic according to any one of Items A19-A26 or Items B26-B31) delivers behavioral therapy, such as cognitive behavioral therapy.
7b″. The combination for use according to embodiment 6b″, wherein the depressive disorder associated with multiple sclerosis is depressive disorder due to a medical condition or major depressive disorder, in particular major depressive disorder.
8b″. The combination for use according to any one of embodiments 1b″ to 7b″, wherein the digital-therapeutic is system-prompted or on-demand.
9b″. The combination for use according to any one of embodiments 1b″ to 8b″, wherein the digital-therapeutic comprises a mobile device, for example selected from the group consisting of a smartphone, a laptop computer, a tablet computer and a wearable computer; in particular a smartphone.
10b″. The combination for use according to any one of embodiments 1b″ to 9b″, which comprises one or more further active agent (i.e. at least one further active agent).
11b″. The combination for use according to embodiment 10b″, wherein the antidepressant and the one or more active agent (i.e. at least one further active agent) is in the form of a fixed pharmaceutical combination or a non-fixed pharmaceutical combination.
12b″. The combination for use according to embodiments 10b″ or 11b″, wherein the further active agent is selected from the group consisting of a disease-modifying therapy for multiple sclerosis and an anxiolytic.
13b″. The combination for use according to any one of embodiments 1b″ to 11b″, wherein the behavioral therapy is cognitive behavioral therapy for depression associated with multiple sclerosis.
14b″. The combination for use according to embodiment 13b″, wherein the cognitive behavioral therapy for depression associated with multiple sclerosis focuses on mood management and coping with symptoms of multiple sclerosis.
15b″. The combination for use according to embodiment 13b″, wherein the cognitive behavioral therapy for depression associated with multiple sclerosis focuses on mood management or coping with symptoms of multiple sclerosis.
16b″. The combination for use according to any one of embodiments 1b″ to 12b″, wherein the behavioral therapy is cognitive behavioral therapy for depressive disorder associated with multiple sclerosis.
17b″. The combination for use according to embodiment 16b″, wherein the cognitive behavioral therapy for depressive disorder associated with multiple sclerosis focuses on mood management and coping with symptoms of multiple sclerosis.
18b″. The combination for use according to embodiment 16b″, wherein the cognitive behavioral therapy for depressive disorder associated with multiple sclerosis focuses mood management or coping with symptoms of multiple sclerosis.
19b″. The combination for use according to any one of embodiments 1b″ to 18b″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (c″)

1c″. An antidepressant for use in the treatment of depression associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
2c″. An antidepressant for use in reducing depressive symptoms associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
3c″. The antidepressant for use according to embodiment 2c″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4c″. The antidepressant for use according to embodiment 1c″, wherein depression associated with multiple sclerosis is selected from the group consisting of mild depression associated with multiple sclerosis, moderate depression associated with multiple sclerosis or severe depression associated with multiple sclerosis.
5c″. The antidepressant for use according to embodiment 4c″, wherein mild depression, moderate depression and severe depression are characterized according to the total score of depressive symptom severity of the Montgomery-Åsberg Depression Rating Scale or of the Beck Depression Inventory.
6c″. The antidepressant for multiple sclerosis for use in the treatment of depressive disorder associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
7c″. The antidepressant for use according to embodiment 6c″, wherein the depressive disorder associated with multiple sclerosis is depressive disorder due to a medical condition or major depressive disorder, in particular major depressive disorder.
8c″. The antidepressant for use according to any one of embodiments 1c″ to 7c″, wherein the computer-implemented behavioral therapy, such as computer-implemented cognitive behavioral therapy, is system-prompted or on-demand.
9c″. The antidepressant for use according to any one of embodiments 1c″ to 7c″, wherein the computer-implemented behavioral therapy is provided by a computerized device.
10c″. The antidepressant for use according to embodiment 9c″, wherein the computerized device is a mobile device.
11c″. The antidepressant for use according to embodiment 10c″, wherein the mobile device is selected from the group consisting of a smartphone, a laptop computer, a tablet computer, and a wearable computer; in particular a smartphone.
12c″. The antidepressant for use according to any one of embodiments 1c″ to 11c″, wherein the antidepressant is in the form of a pharmaceutical combination comprising one or more further active agent (i.e. at least one further active agent).
13c″. The antidepressant for use according to embodiment 12c″, wherein the pharmaceutical combination is a fixed pharmaceutical combination or a non-fixed pharmaceutical combination.
14c″. The antidepressant for use according to embodiments 12c″ or 13c″, wherein the one or more active agent (i.e. at least one further active agent) is selected from the group consisting of a disease-modifying therapy for multiple sclerosis and an anxiolytic.
15c″. The antidepressant for use according to any one of embodiments 1c″ to 14c″, wherein the behavioral therapy is cognitive behavioral therapy for depression associated with multiple sclerosis.
16c″. The antidepressant for use according to embodiment 15c″, wherein the cognitive behavioral therapy for depression associated with multiple sclerosis focuses on mood management and coping with symptoms of multiple sclerosis.
17c″. The antidepressant for use according to embodiment 15c″, wherein the cognitive behavioral therapy for depression associated with multiple sclerosis focuses on mood management or coping with symptoms of multiple sclerosis.
18c″. The antidepressant for use according to any one of embodiments 1c″ to 14c″, wherein the behavioral therapy is cognitive behavioral therapy for depressive disorder associated with multiple sclerosis.
19c″. The antidepressant for use according to embodiment 18c″, wherein the cognitive behavioral therapy for depressive disorder associated with multiple sclerosis focuses on mood management and coping with symptoms of multiple sclerosis.
20c″. The antidepressant for use according to embodiment 18c″, wherein the cognitive behavioral therapy for depressive disorder associated with multiple sclerosis focuses on mood management or coping with symptoms of multiple sclerosis.
21c″. The antidepressant for use according to any one of embodiments 1c″ to 20c″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (d″)

1d″. Use of an antidepressant for the manufacture of a medicament for the treatment of depression associated with multiple sclerosis, wherein
i) the medicament is provided in combination with a computer-implemented device; and
ii) the computer-implemented device is an electronic device according to any one of Items A1-A7, A18 or Items B1-B8, B22-B25.
   2d″. Use of an antidepressant for the manufacture of a medicament for a treatment to reduce depressive symptoms associated with multiple sclerosis, wherein
i) the medicament is provided in combination with a computer-implemented device; and ii) the computer-implemented device is an electronic device according to any one of Items A1-A7, A18 or Items B1-B8, B22-B25.
   3d″. The use according to embodiment 2d″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4d″. The use according to embodiment 1d″, wherein depression associated with multiple sclerosis is selected from the group consisting of mild depression associated with multiple sclerosis, moderate depression associated with multiple sclerosis or severe depression associated with multiple sclerosis.
   5d″. The use according to embodiment 4d″, wherein mild depression, moderate depression and severe depression are characterized according to the total score of depressive symptom severity of the Montgomery-Åsberg Depression Rating Scale or of the Beck Depression Inventory.
   6d″. Use of an antidepressant for the manufacture of a medicament for the treatment of depressive disorder associated with multiple sclerosis, wherein
i) the medicament is provided in combination with a computer-implemented device; and
ii) the computer-implemented device is an electronic device according to any one of Items A1-A7, A18 or Items B1-B8, B22-B25.
   7d″. The use according to embodiment 6d″, wherein the depressive disorder associated with multiple sclerosis is depressive disorder due to a medical condition or major depressive disorder, in particular major depressive disorder.
   8d″. The use according to any one of embodiments 1d″ to 7d″, wherein the computer-implemented behavioral therapy, such as computer-implemented cognitive behavioral therapy, is system-prompted or on-demand.
   9d″. The use according to any one of embodiments 1d″ to 8d″, wherein the computer-implemented device is a computer-implemented mobile device, for example selected from the group consisting of a smartphone, a laptop computer, a tablet computer and a wearable computer; in particular a smartphone.
   10d″. The use according to any one of embodiments 1d″ to 9d″, which comprises one or more further active agent (i.e. at least one further active agent).
   11d″. The use according to embodiment 10d″, wherein the antidepressant and the one or more active agent (i.e. at least one further active agent) is in the form of a fixed pharmaceutical combination or a non-fixed pharmaceutical combination.
   12d″. The use according to embodiments 10d″ or 11d″, wherein the further active agent is selected from the group consisting of a disease-modifying therapy for multiple sclerosis and an anxiolytic.
   13d″. The use according to any one of embodiments 1d″ to 11d″, wherein the computer-implemented device delivers behavioral therapy, such as cognitive behavioral therapy.
   14d″. The use according to embodiment 13d″, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
   15d″. The use according to embodiment 13d″, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
   16d″. The use according to any one of embodiments 1d″ to 12d″, wherein the behavioral therapy is cognitive behavioral therapy for depressive disorder associated with multiple sclerosis.
   17d″. The use according to embodiment 16d″, wherein the cognitive behavioral therapy for depressive disorder associated with multiple sclerosis focuses on mood management and coping with symptoms of multiple sclerosis.
   18d″. The use according to embodiment 16d″, wherein the cognitive behavioral therapy for depressive disorder associated with multiple sclerosis focuses on mood management or coping with symptoms of multiple sclerosis.
   19d″. The use according to any one of embodiments 1d″ to 18d″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (e″)

1e″. Use of an antidepressant for the manufacture of a medicament for the treatment of depression associated with multiple sclerosis, wherein
i) the medicament is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   2e″. Use of an antidepressant for the manufacture of a medicament for a treatment to reduce depressive symptoms associated with multiple sclerosis, wherein
i) the medicament is provided in combination with a digital-therapeutic (e.g., a digital therapeutic according to any one of Items A19-A26 or Items B26-B31); and
   the digital-therapeutic delivers behavioral therapy, such as cognitive behavioral therapy.
   3e″. The use according to embodiment 2e″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4e″. The use according to embodiment 1e″, wherein depression associated with multiple sclerosis is selected from the group consisting of mild depression associated with multiple sclerosis, moderate depression associated with multiple sclerosis or severe depression associated with multiple sclerosis.
   5e″. The use according to embodiment 4e″, wherein mild depression, moderate depression and severe depression are characterized according to the total score of depressive symptom severity of the Montgomery-Åsberg Depression Rating Scale or of the Beck Depression Inventory.
   6e″. Use of an antidepressant for the manufacture of a medicament for the treatment of depressive disorder associated with multiple sclerosis, wherein
iii) the medicament is provided in combination with a digital-therapeutic (e.g., a digital therapeutic according to any one of Items A19-A26 or Items B26-B31); and
iv) the digital-therapeutic delivers behavioral therapy, such as cognitive behavioral therapy.
   7e″. The use according to embodiment 6e″, wherein the depressive disorder associated with multiple sclerosis is depressive disorder due to a medical condition or major depressive disorder, in particular major depressive disorder.
   8e″. The use according to any one of embodiments 1e″ to 7e″, wherein the digital-therapeutic is system-prompted or on-demand.
   9e″. The use according to any one of embodiments 1e″ to 10e″, wherein the digital-therapeutic comprises a mobile device, for example selected from the group consisting of a smartphone, a laptop computer, a tablet computer, and a wearable computer; in particular a smartphone.
   10e″. The use according to any one of embodiments 1e″ to 13e″, wherein the antidepressant is in the form of a pharmaceutical combination comprising one or more further active agent (i.e. at least one further active agent).
   11e″. The use according to embodiment 14e″, wherein the pharmaceutical combination is a fixed pharmaceutical combination or a non-fixed pharmaceutical combination.
   12e″. The use according to embodiments 14e″ or 15e″, wherein the one or more active agent (i.e. at least one further active agent) is selected from the group consisting of a disease-modifying therapy for multiple sclerosis and an anxiolytic.
   13e″. The use according to any one of embodiments 1e″ to 12e″, wherein the behavioral therapy is cognitive behavioral therapy for depression associated with multiple sclerosis.
   14e″. The use according to embodiment 13e″, wherein the cognitive behavioral therapy for depression associated with multiple sclerosis focuses on mood management and coping with symptoms of multiple sclerosis.
   15e″. The use according to embodiment 13e″, wherein the cognitive behavioral therapy for depression associated with multiple sclerosis focuses on mood management or coping with symptoms of multiple sclerosis.
   16e″. The use according to any one of embodiments 1e″ to 12e″, wherein the behavioral therapy is cognitive behavioral therapy for depressive disorder associated with multiple sclerosis.
   17e″. The use according to embodiment 16e″, wherein the cognitive behavioral therapy for depressive disorder associated with multiple sclerosis focuses on mood management and coping with symptoms of multiple sclerosis.
   18e″. The use according to embodiment 16e″, wherein the cognitive behavioral therapy for depressive disorder associated with multiple sclerosis focuses on mood management or coping with symptoms of multiple sclerosis.
   19e″. The use according to any one of embodiments 1e″ to 18e″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (f″)

1f″. Use of an antidepressant for the manufacture of a medicament for the treatment of depression associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
2f″. Use of an antidepressant for the manufacture of a medicament for a treatment to reduce depressive symptoms associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
3f″. The use according to embodiment 2f″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4f″. The use according to embodiment 1f″, wherein depression associated with multiple sclerosis is selected from the group consisting of mild depression associated with multiple sclerosis, moderate depression associated with multiple sclerosis or severe depression associated with multiple sclerosis.
5f″. The use according to embodiment 4f″, wherein mild depression, moderate depression and severe depression are characterized according to the total score of depressive symptom severity of the Montgomery-Åsberg Depression Rating Scale or of the Beck Depression Inventory.
6f″. Use of an antidepressant for the manufacture of a medicament for the treatment of depressive disorder associated with multiple sclerosis, wherein the use is combined with computer-implemented behavioral therapy (e.g., a method according to any one of Items A8-A17, A29-A31 or Items B9-B21), such as computer-implemented cognitive behavioral therapy.
7f″. The use according to embodiment 6f″, wherein the depressive disorder associated with multiple sclerosis is depressive disorder due to a medical condition or major depressive disorder, in particular major depressive disorder.
8f″. The use according to any one of embodiments 1f″ to 7f″, wherein the computer-implemented behavioral therapy, such as computer-implemented cognitive behavioral therapy.
9f″. The use according to any one of embodiments 1f″ to 7f″, wherein the computer-implemented behavioral therapy is provided by a computerized device.
10f″. The use according to embodiment 9f″, wherein the computerized device is a mobile device.
11F. The use according to embodiment 10f″, wherein the mobile device is selected from the group consisting of a smartphone, a laptop computer, a tablet computer, and a wearable computer; in particular a smartphone.
12f″. The use according to any one of embodiments 1f″ to 11f″, wherein the antidepressant is in the form of a pharmaceutical combination comprising one or more further active agent (i.e. at least one further active agent).
13f″. The use according to embodiment 12f″, wherein the pharmaceutical combination is a fixed pharmaceutical combination or a non-fixed pharmaceutical combination.
14f″. The use according to embodiments 12f″ or 13f″, wherein the one or more active agent (i.e. at least one further active agent) is selected from the group consisting of a disease-modifying therapy for multiple sclerosis and an anxiolytic.
15f″. The use according to any one of embodiments 1f″ to 14f″, wherein the behavioral therapy is cognitive behavioral therapy for depression associated with multiple sclerosis.
16f″. The use according to embodiment 15f″, wherein the cognitive behavioral therapy for depression associated with multiple sclerosis focuses on mood management and coping with symptoms of multiple sclerosis.
17f″. The use according to embodiment 15f″, wherein the cognitive behavioral therapy for depression associated with multiple sclerosis focuses on mood management or coping with symptoms of multiple sclerosis.
18f″. The use according to any one of embodiments 1f″ to 14f″, wherein the behavioral therapy is cognitive behavioral therapy for depressive disorder associated with multiple sclerosis.
19f″. The use according to embodiment 18f″, wherein the cognitive behavioral therapy for depressive disorder associated with multiple sclerosis focuses on mood management and coping with symptoms of multiple sclerosis.
20f″. The use according to embodiment 18f″, wherein the cognitive behavioral therapy for depressive disorder associated with multiple sclerosis focuses on mood management or coping with symptoms of multiple sclerosis.
21f″. The use according to any one of embodiments 1f″ to 20f″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (g″)

1g″. A method for the treatment of depression associated with multiple sclerosis, in a subject, in need thereof, comprising administering to said subject an effective amount of an antidepressant, wherein
i) the an antidepressant is provided in combination with a computer-implemented device; and
ii) the computer-implemented device is an electronic device according to any one of Items A1-A7, A18 or Items B1-B8, B22-B25.
   2g″. A method for a treatment to reduce depressive symptoms associated with multiple sclerosis, in a subject, in need thereof, comprising administering to said subject an effective amount of an antidepressant, wherein
i) the an antidepressant is provided in combination with a computer-implemented device; and
ii) the computer-implemented device is an electronic device according to any one of Items A1-A7, A18 or Items B1-B8, B22-B25.
   3g″. The method according to embodiment 2g″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4g″. The method according to embodiment 1g″, wherein depression associated with multiple sclerosis is selected from the group consisting of mild depression associated with multiple sclerosis, moderate depression associated with multiple sclerosis or severe depression associated with multiple sclerosis.
   5g″. The method according to embodiment 4g″, wherein mild depression, moderate depression and severe depression are characterized according to the total score of depressive symptom severity of the Montgomery-Åsberg Depression Rating Scale or of the Beck Depression Inventory.
   6g″. A method for the treatment of depressive disorder associated with multiple sclerosis, in a subject, in need thereof, comprising administering to said subject an effective amount of an anxiolytic, wherein
i) the an anxiolytic is provided in combination with a computer-implemented device; and
ii) the computer-implemented device is an electronic device according to any one of Items A1-A7, A18 or Items B1-B8, B22-B25.
   7g″. The method according to embodiment 6g″, wherein the depressive disorder associated with multiple sclerosis is depressive disorder due to a medical condition or major depressive disorder, in particular major depressive disorder.
   8g″. The method according to any one of embodiments 1g″ to 7g″, wherein the computer-implemented behavioral therapy, such as computer-implemented cognitive behavioral therapy, is system-prompted or on-demand.
   9g″. The method according to any one of embodiments 1g″ to 8g″, wherein the computer-implemented device is a computer-implemented mobile device, for example selected from the group consisting of a smartphone, a laptop computer, a tablet computer and a wearable computer; in particular a smartphone.
   10g″. The method according to any one of embodiments 1g″ to 9g″, which comprises one or more further active agent (i.e. at least one further active agent).
   11g″. The method according to embodiment 10g″, wherein the antidepressant and the one or more active agent (i.e. at least one further active agent) is in the form of a fixed pharmaceutical combination or a non-fixed pharmaceutical combination.
   12g″. The method according to embodiments 10g″ or 11g″, wherein the further active agent is selected from the group consisting of a disease-modifying therapy for multiple sclerosis and an anxiolytic.
   13g″. The method according to any one of embodiments 1g″ to 11g″, wherein the computer-implemented device delivers behavioral therapy, such as cognitive behavioral therapy.
   14g″. The method according to embodiment 13g″, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
   15g″. The method according to embodiment 13g″, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
   16g″. The method according to any one of embodiments 1g″ to 12g″, wherein the behavioral therapy is cognitive behavioral therapy for depressive disorder associated with multiple sclerosis.
   17g″. The method according to embodiment 16g″, wherein the cognitive behavioral therapy for depressive disorder associated with multiple sclerosis focuses on mood management and coping with symptoms of multiple sclerosis.
   18g″. The method according to embodiment 16g″, wherein the cognitive behavioral therapy for depressive disorder associated with multiple sclerosis focuses on mood management or coping with symptoms of multiple sclerosis.
   19g″. The method according to any one of embodiments 1g″ to 18g″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (h″)

1h″. A method for the treatment of depression associated with multiple sclerosis, in a subject, in need thereof, comprising administering to said subject an effective amount of an antidepressant, wherein
i) the antidepressant is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   2h″. A method for a treatment to reduce depressive symptoms associated with multiple sclerosis, in a subject, in need thereof, comprising administering to said subject an effective amount of an antidepressant, wherein
i) the antidepressant is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   3h″. The method according to embodiment 2h″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
   4h″. The method according to embodiment 1h″, wherein depression associated with multiple sclerosis is selected from the group consisting of mild depression associated with multiple sclerosis, moderate depression associated with multiple sclerosis or severe depression associated with multiple sclerosis.
   5h″. The method according to embodiment 4h″, wherein mild depression, moderate depression and severe depression are characterized according to the total score of depressive symptom severity of the Montgomery-Åsberg Depression Rating Scale or of the Beck Depression Inventory.
   6h″. A method for the treatment of depressive disorder associated with multiple sclerosis, in a subject, in need thereof, comprising administering to said subject an effective amount of an antidepressant, wherein
i) the antidepressant is provided in combination with a digital-therapeutic (e.g., a digital therapeutic according to any one of Items A19-A26 or Items B26-B31); and
ii) the digital-therapeutic delivers behavioral therapy, such as cognitive behavioral therapy.
   7h″. The method according to embodiment 6h″, wherein the depressive disorder associated with multiple sclerosis is depressive disorder due to a medical condition or major depressive disorder, in particular major depressive disorder.
   8h″. The use according to any one of embodiments 1h″ to 7h″, wherein the digital-therapeutic is system-prompted or on-demand.
   9h″. The method according to any one of embodiments 1h″ to 10h″, wherein the digital-therapeutic comprises a mobile device, for example selected from the group consisting of a smartphone, a laptop computer, a tablet computer, and a wearable computer; in particular a smartphone.
   10h″. The method according to any one of embodiments 1h″ to 13h″, wherein the antidepressant is in the form of a pharmaceutical combination comprising one or more further active agent (i.e. at least one further active agent).
   11h″. The method according to embodiment 14h″, wherein the pharmaceutical combination is a fixed pharmaceutical combination or a non-fixed pharmaceutical combination.
   12h″. The method according to embodiments 14h″ or 15h″, wherein the one or more active agent (i.e. at least one further active agent) is selected from the group consisting of a disease-modifying therapy for multiple sclerosis and an anxiolytic.
   13h″. The method according to any one of embodiments 1h″ to 12h″, wherein the behavioral therapy is cognitive behavioral therapy for depression associated with multiple sclerosis.
   14h″. The method according to embodiment 13h″, wherein the cognitive behavioral therapy for depression associated with multiple sclerosis focuses on mood management and coping with symptoms of multiple sclerosis.
   15h″. The method according to embodiment 13h″, wherein the cognitive behavioral therapy for depression associated with multiple sclerosis focuses on mood management or coping with symptoms of multiple sclerosis.
   16h″. The method according to any one of embodiments 1h″ to 12h″, wherein the behavioral therapy is cognitive behavioral therapy for depressive disorder associated with multiple sclerosis.
   17h″. The method according to embodiment 16h″, wherein the cognitive behavioral therapy for depressive disorder associated with multiple sclerosis focuses on mood management and coping with symptoms of multiple sclerosis.
   18h″. The method according to embodiment 16h″, wherein the cognitive behavioral therapy for depressive disorder associated with multiple sclerosis focuses on mood management or coping with symptoms of multiple sclerosis.
   19h″. The method according to any one of embodiments 1h″ to 18h″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (j″)

1j″. A method for the treatment of depression associated with multiple sclerosis, in a subject, in need thereof, comprising administering to said subject an effective amount of an antidepressant, wherein the method is combined with computer-implemented behavioral therapy (e.g., a method according to any one of Items A8-A17, A29-A31 or Items B9-B21), such as computer-implemented cognitive behavioral therapy.
2j″. A method for a treatment to reduce depressive symptoms/associated with multiple sclerosis, in a subject, in need thereof, comprising administering to said subject an effective amount of an antidepressant, wherein the method is combined with computer-implemented behavioral therapy (e.g., a method according to any one of Items A8-A17, A29-A31 or Items B9-B21), such as computer-implemented cognitive behavioral therapy.
3j″. The method according to embodiment 2j″, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4j″. The method according to embodiment 1j″, wherein depression associated with multiple sclerosis is selected from the group consisting of mild depression associated with multiple sclerosis, moderate depression associated with multiple sclerosis or severe depression associated with multiple sclerosis.
5j″. The method according to embodiment 4j″, wherein mild depression, moderate depression and severe depression are characterized according to the total score of depressive symptom severity of the Montgomery-Åsberg Depression Rating Scale or of the Beck Depression Inventory.
6j″. A method for the treatment of depressive disorder associated with multiple sclerosis, in a subject, in need thereof, comprising administering to said subject an effective amount of an antidepressant, wherein the method is combined with computer-implemented behavioral therapy (e.g., a method according to any one of Items A8-A17, A29-A31 or Items B9-B21), such as computer-implemented cognitive behavioral therapy.
7j″. The method according to embodiment 6j″, wherein the depressive disorder associated with multiple sclerosis is depressive disorder due to a medical condition or major depressive disorder, in particular major depressive disorder.
8j″. The method according to any one of embodiments 1j″ to 7j″, wherein the computer-implemented behavioral therapy, such as computer-implemented cognitive behavioral therapy, is system-prompted or on-demand.
9j″. The method according to any one of embodiments 1j″ to 7j″, wherein the computer-implemented behavioral therapy is provided by a computerized device.
10j″. The method according to embodiment 9j″, wherein the computerized device is a mobile device.
11j″. The method according to embodiment 10j″, wherein the mobile device is selected from the group consisting of a smartphone, a laptop computer, a tablet computer, and a wearable computer; in particular a smartphone.
12j″. The method according to any one of embodiments 1j″ to 11j″, wherein the antidepressant is in the form of a pharmaceutical combination comprising one or more further active agent (i.e. at least one further active agent).
13j″. The method according to embodiment 12j″, wherein the pharmaceutical combination is a fixed pharmaceutical combination or a non-fixed pharmaceutical combination.
14j″. The method according to embodiments 12j″ or 13j″, wherein the one or more active agent (i.e. at least one further active agent) is selected from the group consisting of a disease-modifying therapy for multiple sclerosis and an anxiolytic.
15j″. The method according to any one of embodiments 1j″ to 14j″, wherein the behavioral therapy is cognitive behavioral therapy for depression associated with multiple sclerosis.
16j″. The method according to embodiment 15j″, wherein the cognitive behavioral therapy for depression associated with multiple sclerosis focuses on mood management and coping with symptoms of multiple sclerosis.
17j″. The method according to embodiment 15j″, wherein the cognitive behavioral therapy for depression associated with multiple sclerosis focuses on mood management or coping with symptoms of multiple sclerosis.
18j″. The method according to any one of embodiments 1j″ to 14j″, wherein the behavioral therapy is cognitive behavioral therapy for depressive disorder associated with multiple sclerosis.
19j″. The method according to embodiment 18j″, wherein the cognitive behavioral therapy for depressive disorder associated with multiple sclerosis focuses on mood management and coping with symptoms of multiple sclerosis.
20j″. The method according to embodiment 18j″, wherein the cognitive behavioral therapy for depressive disorder associated with multiple sclerosis focuses on mood management or coping with symptoms of multiple sclerosis.
21j″. The method according to any one of embodiments 1j″ to 20j″, wherein multiple sclerosis is selected from the group consisting of relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

Other embodiments of the present disclosure are:

### Embodiments (a*)-(c*), Related to the Medical Used of the Digital Application

### Embodiments (a*)

1a*. A computer-implemented device as defined herein for use in the treatment of depression associated with multiple sclerosis.
2a*. A computer-implemented device as defined herein for use in reducing depressive symptoms associated with multiple sclerosis.
3a*. The computer-implemented device for use according to embodiment 2a*, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4a*. The computer-implemented device for use according to embodiment 1a*, wherein depression associated with multiple sclerosis is selected from the group consisting of mild depression associated with multiple sclerosis, moderate depression associated with multiple sclerosis or severe depression associated with multiple sclerosis.
5a*. The computer-implemented device for use according to embodiment 4a*, wherein mild depression, moderate depression and severe depression are characterized according to the total score of depressive symptom severity of the Montgomery-Åsberg Depression Rating Scale or of the Beck Depression Inventory.
6a*. A computer-implemented device as defined herein for use in the treatment of depressive disorder associated with multiple sclerosis.
7a*. The computer-implemented device for use according to embodiment 6a*, wherein the depressive disorder associated with multiple sclerosis is depressive disorder due to a medical condition or major depressive disorder, in particular major depressive disorder.
8a*. The computer-implemented device for use according to any one of embodiments 1a* to 7a*, wherein the computer-implemented device is a computer-implemented mobile device, for example selected from the group consisting of a smartphone, a laptop computer, a tablet computer and a wearable computer; in particular a smartphone.
9a*. The computer-implemented device for use according to any one of embodiments 1a* to 8a*, wherein the computer-implemented device is system-prompted or on-demand.
10a*. The computer-implemented device for use according to any one of embodiments 1a* to 9a*, wherein the computer-implemented device delivers behavioral therapy, such as cognitive behavioral therapy.
11a*. The computer-implemented device for use according to embodiment 10a*, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
12a*. The computer-implemented device for use according to embodiment 10a*, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
13a*. The computer-implemented device for use according to any one of embodiments 1a* to 12a*, wherein the behavioral therapy is cognitive behavioral therapy for depressive disorder associated with multiple sclerosis.
14a*. The computer-implemented device for use according to embodiment 13a*, wherein the cognitive behavioral therapy for depressive disorder associated with multiple sclerosis focuses on mood management and coping with symptoms of multiple sclerosis.
15a*. The computer-implemented device for use according to embodiment 13a*, wherein the cognitive behavioral therapy for depressive disorder associated with multiple sclerosis focuses on mood management or coping with symptoms of multiple sclerosis.
16a*. The computer-implemented device for use according to any one of embodiments 1a* to 15a*, wherein multiple sclerosis is selected from relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (b*)

1b*. A method for the treatment of depression associated with multiple sclerosis, in a subject, in need thereof, comprising administering to said subject behavioral therapy provided by a computer-implemented device as defined herein.
2b*. A method for a treatment to reduce depressive symptoms associated with multiple sclerosis, in a subject, in need thereof, comprising administering to said subject behavioral therapy provided by a computer-implemented device as defined herein.
3b*. The method according to embodiment 2b*, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4b*. The method according to embodiment 1b*, wherein depression associated with multiple sclerosis is selected from the group consisting of mild depression associated with multiple sclerosis, moderate depression associated with multiple sclerosis or severe depression associated with multiple sclerosis.
5b*. The method according to embodiment 4b*, wherein mild depression, moderate depression and severe depression are characterized according to the total score of depressive symptom severity of the Montgomery-Åsberg Depression Rating Scale or of the Beck Depression

Inventory.

6b*. A method for the treatment of depressive disorder associated with multiple sclerosis, in a subject, in need thereof, comprising administering to said subject behavioral therapy provided by a computer-implemented device as defined herein.
7b*. The method according to embodiment 6b*, wherein the depressive disorder associated with multiple sclerosis is depressive disorder due to a medical condition or major depressive disorder, in particular major depressive disorder.
8b*. The method according to any one of embodiments 1b* to 7b*, wherein the computer-implemented device is a computer-implemented mobile device, for example selected from the group consisting of a smartphone, a laptop computer, a tablet computer and a wearable computer; in particular a smartphone.
9b*. The method according to any one of embodiments 1b* to 8b*, wherein the computer-implemented device is system-prompted or on-demand.
10b*. The method according to any one of embodiments 1b* to 9b*, wherein the behavioral therapy is cognitive behavioral therapy.
11b*. The method according to embodiment 10b*, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
12b*. The method according to embodiment 10b*, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
13b*. The method according to any one of embodiments 1b* to 12b*, wherein the behavioral therapy is cognitive behavioral therapy for depressive disorder associated with multiple sclerosis.
14b*. The method according to embodiment 13b*, wherein the cognitive behavioral therapy for depressive disorder associated with multiple sclerosis focuses on mood management and coping with symptoms of multiple sclerosis.
15b*. The method according to embodiment 13b*, wherein the cognitive behavioral therapy for depressive disorder associated with multiple sclerosis focuses on mood management or coping with symptoms of multiple sclerosis.
16b*. The method according to any one of embodiments 1b* to 15b*, wherein multiple sclerosis is selected from relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### Embodiments (c*)

1c*. Use of a computer-implemented device as defined herein for the treatment of depression associated with multiple sclerosis.
2c*. Use of a computer-implemented device as defined herein for a treatment to reduce depressive symptoms associated with multiple sclerosis.
3c*. The use according to embodiment 2c*, wherein the depressive symptoms are one or more depressive symptoms selected from the group according to the Montgomery-Åsberg Depression Rating Scale, the Beck Depression Inventory or the Patient Healthy Questionnaire-9 (PHQ-9).
4c*. The use according to embodiment 1c*, wherein depression associated with multiple sclerosis is selected from the group consisting of mild depression associated with multiple sclerosis, moderate depression associated with multiple sclerosis or severe depression associated with multiple sclerosis.
5c*. The use according to embodiment 4c*, wherein mild depression, moderate depression and severe depression are characterized according to the total score of depressive symptom severity of the Montgomery-Åsberg Depression Rating Scale or of the Beck Depression Inventory.
6c*. Use of a computer-implemented device as defined herein for the treatment of depressive disorder associated with multiple sclerosis.
7c*. The use according to embodiment 6c*, wherein the depressive disorder associated with multiple sclerosis is depressive disorder due to a medical condition or major depressive disorder, in particular major depressive disorder.
8c*. The use according to any one of embodiments 1c* to 7c*, wherein the computer-implemented device is a computer-implemented mobile device, for example selected from the group consisting of a smartphone, a laptop computer, a tablet computer and a wearable computer; in particular a smartphone.
9c*. The use according to any one of embodiments 1c* to 8c*, wherein the computer-implemented device is system-prompted or on-demand.
10c*. The use according to any one of embodiments 1c* to 9c*, wherein the computer-implemented device delivers behavioral therapy, such as cognitive behavioral therapy.
11c*. The use according to embodiment 10c*, wherein the behavioral therapy is cognitive behavioral therapy for depression associated with multiple sclerosis, such as cognitive behavioral therapy for depression associated with multiple sclerosis that focuses on mood management and coping with symptoms of multiple sclerosis.
12c*. The use according to embodiment 10c*, wherein the behavioral therapy is cognitive behavioral therapy for depression associated with multiple sclerosis, such as cognitive behavioral therapy for depression associated with multiple sclerosis that focuses on mood management or coping with symptoms of multiple sclerosis.
13c*. The use according to any one of embodiments 1c* to 12c*, wherein the behavioral therapy is cognitive behavioral therapy for depressive disorder associated with multiple sclerosis.
14c*. The use according to embodiment 13c*, wherein the cognitive behavioral therapy for depressive disorder associated with multiple sclerosis focuses on mood management and coping with symptoms of multiple sclerosis.
15c*. The use according to embodiment 13c*, wherein the cognitive behavioral therapy for depressive disorder associated with multiple sclerosis focuses on mood management or coping with symptoms of multiple sclerosis.
16c*. The use according to any one of embodiments 1c* to 15c*, wherein multiple sclerosis is selected from relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

### General Terms

The term “multiple sclerosis” or “MS”, as used herein, refers to an autoimmune disease where lymphocytes travel to the central nervous system and attack the myelin sheath covering nerves. Thus, MS relates to a disease of the central nervous system that causes prolonged and severe disability in a subject suffering therefrom. How to diagnose MS is well known in the art, in particular, MS can be diagnosed, for example, with reference to McDonald diagnostic criteria. These criteria were originally introduced in 2001 [McDonald W I, et al., Recommended diagnostic criteria for multiple sclerosis: guidelines from the International Panel on the diagnosis of multiple sclerosis. (2001) Annals of neurology. 50 (1): 121-7], revised in 2005 [Polman C H, Reingold S C, Edan G, Filippi M, Hartung H P, Kappos L, Lublin F D, Metz L M, McFarland H F, et al., Diagnostic criteria for multiple sclerosis: 2005 revisions to the “McDonald Criteria”. (2005) Annals of neurology. 58 (6): 840-6], in 2010 [Polman C H, et al., Diagnostic criteria for multiple sclerosis: 2010 Revisions to the McDonald criteria. (2011) Annals of Neurology. 69 (2): 292], in 2016 [Filippi M, et al., Mill criteria for the diagnosis of multiple sclerosis: MAGNIMS consensus guidelines. (2016) The Lancet. Neurology. 15 (3): 292-303], and in 2017 [Thompson A J, et al., Diagnosis of multiple sclerosis: 2017 revisions of the McDonald criteria. (2017) The Lancet. Neurology], each of which is incorporated herein by reference. According to the 2017 revision, diagnosis of multiple sclerosis can be made if there is fulfillment of either of these five categories of criteria, depending on how many clinical attacks have occurred:
≥2 clinical attacks
   with ≥2 lesions with objective clinical evidence
   with no additional data needed
≥2 clinical attacks
   with 1 lesion with objective clinical evidence and a clinical history suggestive of a previous lesion
   with no additional data needed
≥2 clinical attacks
   with 1 lesion with objective clinical evidence and no clinical history suggestive of a previous lesion
   with dissemination in space evident on Mill
1 clinical attack (i.e. clinically isolated syndrome)
   with ≥2 lesions with objective clinical evidence
   with dissemination in time evident on MRI or demonstration of CSF-specific oligoclonal bands
1 clinical attack (i.e. clinically isolated syndrome)
   with 1 lesion with objective clinical evidence
   with dissemination in space evident on Mill
   with dissemination in time evident on MRI or demonstration of CSF-specific oligoclonal bands

Any reference to the term “multiple sclerosis”, alone or in any expression as used herein, is to be understood to encompass the different subtypes of multiple sclerosis: relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS). In one embodiment, “multiple sclerosis”, alone or in any expression as used herein, refers to relapsing-remitting multiple sclerosis, primary-progressive multiple sclerosis, secondary-progressive multiple sclerosis (e.g. relapsing SPMS and non-relapsing SPMS), progressive-relapsing multiple sclerosis and relapsing multiple sclerosis. In another embodiment, “multiple sclerosis”, alone or in any expression as used herein, refers to relapsing-remitting multiple sclerosis. In yet another embodiment, “multiple sclerosis”, alone or in any expression as used herein, refers to secondary-progressive multiple sclerosis (e.g. relapsing SPMS and non-relapsing SPMS). RRMS is characterized by phases of relapses (characterized by sudden intensification of symptoms), followed by phases of remission. The duration of the phases of relapse and remission vary from a patient to another, a relapse typically lasting form days to weeks, a remission lasting anywhere from several weeks to several years. PPMS is characterized by gradual progression of the disease from onset with no relapses. Similarly, also SPMS is characterized by steadily worsening of symptoms over time, however all SPMS patients have been RRMS patients at MS onset. At an early stage of SPMS, relapses are still superimposed on the progressive worsening (relapsing SPMS), at a later stage, said relapses disappear (non-relapsing SPMS). PRMS is characterized by gradual progression from MS onset, however also with frequent phases of symptomatic exacerbations (i.e. relapses). PRMS also has a progressive disease course from onset, however (in contrast to PPMS) with clear acute relapses. As relapses occur in RRMS, PRMS, and early SPMS, the MS field also uses the term RMS to refer to and differentiate these disease courses from those without relapses (i.e., late SPMS and PPMS). The term “clinically isolated syndrome” (CIS) refers to a first episode of neurologic symptoms that lasts at least 24 hours and is caused by inflammation or demyelination in the central nervous system (CNS).

The term “multiple sclerosis patient” or “patient with multiple sclerosis” refers to a patient diagnosed with multiple sclerosis, as defined herein.

In one embodiment, “multiple sclerosis patient” or “patient with multiple sclerosis” refers to a patient diagnosed with relapsing-remitting multiple sclerosis (RRMS), with primary-progressive multiple sclerosis (PPMS), with secondary-progressive multiple sclerosis (SPMS), such as relapsing SPMS and non-relapsing SPMS, with progressive-relapsing multiple sclerosis (PRMS), with relapsing multiple sclerosis (RMS) or with clinically isolated syndrome (CIS); in particular a patient diagnosed with relapsing-remitting multiple sclerosis (RRMS) or with clinically isolated syndrome (CIS).

In one embodiment, a patient diagnosed with multiple sclerosis, as defined herein, is also diagnosed with “depressive disorder”, for example, as defined with reference to DSM-5 criteria (i.e. according to the Diagnostic and Statistical Manual of Mental Disorders. 5^{th} Edition, Washington, D.C.: American Psychiatric Association, 2013). In one embodiment, “depressive disorder” is selected from “depressive disorder due to a medical condition” and “major depressive disorder”, for example, as defined with reference to DSM-5 criteria.

The term “depression associated with multiple sclerosis”, as used herein, refers to depressive symptoms associated with multiple sclerosis (i.e. depressive symptoms in a patient with multiple sclerosis). In one embodiment, a patient diagnosed with multiple sclerosis, as defined herein, has moderate depression, for example as measured by the Montgomery-Åsberg Depression Rating Scale (MADRS), for example has a MADRS total score ≥20; in particular a patient diagnosed with relapsing-remitting multiple sclerosis (RRMS) or with clinically isolated syndrome (CIS) having moderate depression, such as a patient diagnosed with relapsing-remitting multiple sclerosis (RRMS) or with clinically isolated syndrome (CIS) having a Montgomery-Åsberg Depression Rating Scale (MADRS) total score ≥20. Depressive symptoms are well known in the art, in particular with reference clinical scales, such as the Montgomery-Åsberg Depression Rating Scale (MADRS) [Original reference: Br. J. Psychiat. (1979), 134, 382-389], the Beck Depression Inventory (e.g. BDI-II) [Beck, A. T., Brown, G. K. (1996), Manual for the Beck Depression Inventory-II, San Antonio, Tex.: Psychological Corporation] or the Patient Healthy Questionnaire-9 (e.g. in Kroenke K., et al., J Gen Intern Med, 2001, 16, 606-613, which are incorporated herein by reference].

In a first embodiment, “depression associated with multiple sclerosis”, as used herein, refers to depressive symptoms selected from the group consisting of sadness, pessimism, failure feelings, loss of pleasure, guilty feelings, punishment feelings, self-dislike, self-criticalness, suicidal thoughts or wishes, crying, agitation, loss of interest, indecisiveness, worthlessness, loss of energy, changes in sleep pattern, irritability, changes in appetite, concentration difficulty, tiredness or fatigue, and loss of interest in sex; in a patient with multiple sclerosis. In a second embodiment, “depression associated with multiple sclerosis”, as used herein, refers to depressive symptoms selected from the group consisting of sadness (e.g. apparent sadness, reported sadness), inner tension, reduced sleep, reduced appetite, concentration difficulties, lassitude, inability to feel, pessimistic thoughts and suicidal thoughts; in a patient with multiple sclerosis. In a third embodiment, “depression associated with multiple sclerosis”, as used herein, refers to depressive symptoms selected from the group consisting of: 1) loss of interest or pleasure; 2) feeling down; 3) changes in sleep pattern; 4) 20) tiredness or little energy 5) change in appetite; 6) failure feelings; 7) concentration difficulties, 8) moving or speaking slowly or restless, and 9) punishment feelings; in a patient with multiple sclerosis.

In another embodiment, the term “depression associated with multiple sclerosis”, as used herein refers to mild depression associated with multiple sclerosis, moderate depression associated with multiple sclerosis and severe depression associated with multiple sclerosis; namely, mild depression, moderate depression and severe depression, in a patient with multiple sclerosis, wherein the terms mild, moderate and severe refer to the severity of depressive symptoms. How to determine the severity of depressive symptoms is well known in the art, in particular with reference to a stablished diagnostic evaluation, that measures the severity of depression according to a total score of depressive symptom severity, such as the Montgomery-Åsberg Depression Rating Scale or the Beck Depression Inventory (e.g. BDI-II).

“BDI-II” is a 21-item self-report instrument for measuring depression severity. It provides a total score index of current depression severity [i.e., minimal depression (0 to 13), mild depression (14 to 19), moderate depression (20 to 28), severe depression (29 to 63)]. It can be administered repeatedly, which can reflect improvement or worsening of depression in response to treatment. Depressive symptoms measured according to this questionnaire are selected from the group consisting of: 1) sadness, 2) pessimism, 3) failure feelings, 4) loss of pleasure, 5) guilty feelings, 6) punishment feelings, 7) self-dislike, 8) self-criticalness, 9) suicidal thoughts or wishes, 10) crying, 11) agitation, 12) loss of interest, 13) indecisiveness, 14) worthlessness, 15) loss of energy, 16) changes in sleep pattern, 17) irritability, 18) changes in appetite, 19) concentration difficulty, 20) tiredness or fatigue, and 21) loss of interest in sex.

“MADRS” is a ten-item structured interview used to measure depression severity. It provides a total score of current depression severity [i.e., normal (0 to 6), mild depression (7 to 19), moderate depression (20 to 34), severe depression (35 to 60)]. It can be administered repeatedly, which can reflect improvement or worsening of depression in response to treatment. Depressive symptoms measured according to this questionnaire are selected from the group consisting of:

1) apparent sadness [e.g., despondency, gloom and despair (more than just ordinary transient low spirits), reflected in speech, facial expression, and posture];

2) reported sadness [e.g., reports of depressed mood, regardless of whether it is reflected in appearance or not. Includes low spirits, despondency or the feeling of being beyond help and without hope];

3) inner tension [e.g., feelings of ill-defined discomfort, edginess, inner turmoil, mental tension mounting to either panic, dread or anguish];

4) reduced sleep [e.g., experience of reduced duration or depth of sleep compared to the subject's own normal pattern when well];

5) reduced appetite [e.g., feeling of a loss of appetite compared with when-well];

6) concentration difficulties [i.e. difficulties in collecting one's thoughts mounting to an incapacitating lack of concentration];

7) lassitude [e.g., difficulty in getting started or slowness in initiating and performing everyday activities];

8) inability to feel [e.g., subjective experience of reduced interest in the surroundings, or activities that normally give pleasure];

9) pessimistic thoughts [e.g., thoughts of guilt, inferiority, self-reproach, sinfulness, remorse and ruin]; and,

10) suicidal thoughts [e.g., the feeling that life is not worth living, that a natural death would be welcome, suicidal thoughts, and preparations for suicide].

The Patient Health Questionnaire-9 “PHQ-9” is a 9-item self-report tool for measuring depression severity. It provides a total score index of current depressive symptom severity [i.e., minimal depression (1 to 4), mild depression (5 to 9), moderate depression (10 to 14), moderately severe depression (15 to 19), severe depression (20 to 27)]. The tool rates the frequency of symptoms which factors into the scoring severity. The PHQ-9 can be administered repeatedly, which can reflect improvement or worsening of depression in response to treatment. Depressive symptoms measured according to this questionnaire are selected, for example, from the group consisting of: 1) loss of interest or pleasure; 2) feeling down; 3) changes in sleep pattern; 4) tiredness or little energy 5) change in appetite; 6) failure feelings; 7) concentration difficulties, 8) moving or speaking slowly or restless, and 9) punishment feelings.

The Multiple Sclerosis Quality of Life-54 (MSQOL-54) is a multidimensional health-related quality of life measure that combines both generic and MS-specific items into a single instrument. (e.g. in Qual Life Res, 1995, 4(3): 187-206).

The term “treatment of depression associated with multiple sclerosis” as used herein, refers, for example, to alleviating depression associated with multiple sclerosis or suppressing depression associated with multiple sclerosis, wherein depression associated with multiple sclerosis is as defined herein. In one embodiment, “treatment of depression associated with multiple sclerosis” refers to decreasing depressive symptoms, such as decreasing intensity, duration or frequency of depressive symptoms, as defined herein, in a multiple sclerosis patient (e.g. in comparison to pre-treatment).

In another embodiment, “treatment of depression associated with multiple sclerosis” as used herein, refers, for example, to “improvement of depression associated with multiple sclerosis” which means “stabilizing the severity of depressive symptoms” or “improving the severity of depressive symptoms”, for example, in comparison to pre-treatment situation. The term “stabilizing the severity of depressive symptoms” refers, for example, to preventing increase in severity of depression (e.g. preventing that mild depression associated with multiple sclerosis, as defined herein, becomes moderate depression associated with multiple sclerosis, as defined herein, or severe depression associated with multiple sclerosis, as defined herein). The term “improving the severity of depressive symptoms” refers to a decrease in severity of depression, as defined herein (e.g. severe depression associated with multiple sclerosis becoming moderate depression associated with multiple sclerosis or moderate depression associated with multiple sclerosis becoming mild depression associated with multiple sclerosis), for example, in comparison to pre-treatment situation. In one embodiment, “treatment of depression associated with multiple sclerosis” refers to decrease of severity of depression associated with multiple sclerosis (e.g. as measured with the Beck Depression Inventory, such as a reduced BDI-II score).

The term “depressive symptom”, as used herein, refers, for example, to one or more (i.e. at least one of) of, sadness, pessimism, failure feelings, loss of pleasure, guilty feelings, punishment feelings, self-dislike, self-criticalness, suicidal thoughts or wishes, crying, agitation, loss of interest, indecisiveness, worthlessness, loss of energy, changes in sleep pattern, irritability, changes in appetite, concentration difficulty, tiredness or fatigue, and loss of interest in sex. Thus, in a first embodiment, a depressive symptom is selected from the group consisting of sadness, pessimism, failure feelings, loss of pleasure, guilty feelings, punishment feelings, self-dislike, self-criticalness, suicidal thoughts or wishes, crying, agitation, loss of interest, indecisiveness, worthlessness, loss of energy, changes in sleep pattern, irritability, changes in appetite, concentration difficulty, tiredness or fatigue, and loss of interest in sex.

In another embodiment, “depressive symptom”, as used herein, refers, for example, to one or more (i.e. at least one of) of, sadness (e.g. apparent sadness, reported sadness), inner tension, reduced sleep, reduced appetite, concentration difficulties, lassitude, inability to feel, pessimistic thoughts and suicidal thoughts. Thus, in a second embodiment, a depressive symptom is selected from the group consisting of sadness (e.g. apparent sadness, reported sadness), inner tension, reduced sleep, reduced appetite, concentration difficulties, lassitude, inability to feel, pessimistic thoughts and suicidal thoughts.

In a further embodiment, “depressive symptom”, as used herein, refers, for example, to one or more of (i.e. at least one of) loss of interest or pleasure, feeling down, changes in sleep pattern, tiredness or little energy, change in appetite, failure feelings, concentration difficulties, moving or speaking slowly or restless, and punishment feelings. Thus, in a further embodiment, a depressive symptom is selected from the group consisting of loss of interest or pleasure, feeling down, changes in sleep pattern, tiredness or little energy, change in appetite, failure feelings, concentration difficulties, moving or speaking slowly or restless, and punishment feelings.

The term “depressive disorder associated with multiple sclerosis”, as used herein refers to depressive disorder in a patient with multiple sclerosis (i.e. a MS with a depressive disorder diagnosis). The term “depressive disorder”, as used herein, is defined, for example, with reference to DSM-5 criteria (i.e. according to the Diagnostic and Statistical Manual of Mental Disorders, 5^{th} Edition, Washington, D.C.: American Psychiatric Association, 2013), the entire contents of which are incorporated herein by reference. The DSM-5 outlines the following criterion to make a diagnosis of depressive disorder [i.e. DMS-5 diagnostic criteria 295.90 (F20.90)]. In one embodiment, the depressive disorder is not caused by MS. In another embodiment, the depressive disorder is caused by MS.

The term “depressive disorder due to a medical condition” as used herein, is defined, for example, with reference to DSM-5 criteria, the entire contents of which are incorporated herein by reference. The DSM-5 outlines the following criterion to make a diagnosis of depressive disorder due to a medical condition [i.e. DMS-5 diagnostic criteria, 293.83]:
A. prominent and persistent period of depressed mood or markedly diminished interest or pleasure in all, or almost all, activities that predominates in the clinical picture.
B. There is evidence from the history, physical examination, or laboratory findings that the disturbance is the direct pathophysiological consequence of another medical condition.
C. The disturbance is not better explained by another mental disorder (e.g., adjustment disorder, with depressed mood, in which the stressor is a serious medical condition).
D. The disturbance does not occur exclusively during the course of a delirium.
E. The disturbance causes clinically significant distress or impairment in social, occupational, or other important areas of functioning.

The term “major depressive disorder” as used herein, is defined, for example, with reference to DSM-5 criteria, the entire contents of which are incorporated herein by reference. The DSM-5 outlines the following criterion to make a diagnosis of depressive disorder due to a medical condition [i.e. DMS-5 diagnostic criteria, 296.20-296.36]:
A. Five (or more) of the following symptoms have been present during the same 2-week period and represent a change from previous functioning; at least one of the symptoms is either (1) depressed mood or (2) loss of interest or pleasure.
   1. Depressed mood most of the day, nearly every day, as indicated by either subjective report (e.g., feels sad, empty, hopeless) or observation made by others (e.g., appears tearful).
   2. Markedly diminished interest or pleasure in all, or almost all, activities most of the day, nearly every day (as indicated by either subjective account or observation).
   3. Significant weight loss when not dieting or weight gain (e.g., a change of more than 5% of body weight in a month), or decrease or increase in appetite nearly every day.
   4. Insomnia or hypersomnia nearly every day.
   5. Psychomotor agitation or retardation nearly every day (observable by others, not merely subjective feelings of restlessness or being slowed down).
   6. Fatigue or loss of energy nearly every day.
   7. Feelings of worthlessness or excessive or inappropriate guilt (which may be delusional) nearly every day (not merely self-reproach or guilt about being sick).
   8. Diminished ability to think or concentrate, or indecisiveness, nearly every day (either by subjective account or as observed by others).
   9. Recurrent thoughts of death (not just fear of dying), recurrent suicidal ideation without a specific plan, or a suicide attempt or a specific plan for committing suicide.
B. The symptoms cause clinically significant distress or impairment in social, occupational, or other important areas of functioning.
C. The episode is not attributable to the physiological effects of a substance or another medical condition.
   Note: Criteria A-C represent a major depressive episode.
D. The occurrence of the major depressive episode is not better explained by schizoaffective disorder, schizophrenia, schizophreniform disorder, delusional disorder, or other specified and unspecified schizophrenia spectrum and other psychotic disorders.
E. There has never been a manic episode or a hypomanic episode.

The following specifiers apply to Major Depressive Disorder:
single episode:
   mild [296.21 (F32.0)]
   moderate [296.22 (F32.1)]
   severe [296.23 (F32.2)]
   with psychotic features [296.24 (F32.3)]
   in partial remission [296.25 (32.4)]
   in full remission [296.26 (F32.5)]
   unspecified [296.20 (F32.9)]
recurrent episode:
   mild [296.31 (F33.0)]
   moderate [296.32 (F33.1)]
   severe [296.33 (F33.2)]
   with psychotic features [296.34 (F33.3)]
   in partial remission [296.35 (33.41)]
   in full remission [296.36 (F33.42)]
   unspecified [296.30 (F33.9)]

The term “disease-modifying therapy for multiple sclerosis”, as used herein, refers to a drug that can modify or change the course of MS (i.e. a disease-modifying drug for multiple sclerosis), such as beta interferons (e.g. interferon beta-1a and interferon beta-1b), monoclonal antibodies (e.g. natalizumab, alemtuzumab, ocrelizumab, rituximab, ofatumumab, ublituximab), immunomodulators (e.g. glatiramer acetate, mitoxantrone, fingolimod, teriflunomide, dimethyl fumarate, masitinib, ozanimod, ponesimod, cladribine, siponimod, ibudilast). In one embodiment, the disease-modifying treatment for multiple sclerosis is selected from the group consisting of a beta interferon (e.g. interferon beta-1a and interferon beta-1b), a monoclonal antibody (e.g. natalizumab, alemtuzumab, ocrelizumab, rituximab, ofatumumab, ublituximab), and an immunomodulator (e.g. glatiramer acetate, mitoxantrone, fingolimod, teriflunomide, dimethyl fumarate, masitinib, ozanimod, ponesimod, cladribine, siponimod, ibudilast). In one embodiment, the disease-modifying treatment for multiple sclerosis is selected from the group consisting of fingolimod, siponimod and ofatumumab. In another embodiment, the disease-modifying treatment for multiple sclerosis is fingolimod. In yet another embodiment, the disease-modifying treatment for multiple sclerosis is siponimod. In a further embodiment, the disease-modifying treatment for multiple sclerosis is In yet another embodiment, the disease-modifying treatment for multiple sclerosis is ofatumumab.

The term “antidepressant”, as used herein, refers to an active ingredient commonly used to treat depression, such as a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g. amitriptyline, nortriptyline, doxepin, desipramine, imipramine, protriptyline, trimipramine, clomipramine), a tetracyclic antidepressant (e.g. maprotiline, mianserin, mirtazapine, setiptiline), or a monoamine oxidase inhibitor (MAOI, e.g. isocarboxazid, phenelzine, selegiline, tranylcypromine). In one embodiment, the antidepressant is selected from the group consisting of a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g. amitriptyline, nortriptyline, doxepin, desipramine, imipramine, protriptyline, trimipramine, clomipramine), a tetracyclic antidepressant (e.g. maprotiline, mianserin, mirtazapine, setiptiline), a monoamine oxidase inhibitor (MAOI, e.g. isocarboxazid, phenelzine, selegiline, tranylcypromine) and hypericum perforatum. In another embodiment, the antidepressant is selected from the group consisting of fluoxetine, citalopram, sertraline, paroxetine, escitalopram, fluvoxamine, vilazodone, vortioxetine, venlafaxine, duloxetine, desvenlafaxine, milnacipran, levomilnacipran, bupropion, amitriptyline, nortriptyline, doxepin, desipramine, imipramine, protriptyline, trimipramine, clomipramine, maprotiline, mianserin, mirtazapine, setiptiline, isocarboxazid, phenelzine, selegiline, tranylcypromine and hypericum perforatum; or salts thereof. In another embodiment, the antidepressant is selected from the group consisting of fluoxetine, citalopram, sertraline, paroxetine, escitalopram, fluvoxamine, vilazodone, vortioxetine, venlafaxine, duloxetine, desvenlafaxine, milnacipran, levomilnacipran, bupropion, amitriptyline, nortriptyline, doxepin, desipramine, imipramine, protriptyline, trimipramine, clomipramine, maprotiline, mianserin, mirtazapine, setiptiline, isocarboxazid, phenelzine, selegiline and tranylcypromine; or salts thereof.

The term “anxiolytic”, as used herein, refers to a drug that inhibits anxiety, such as benzodiazepines (e.g. alprazolam, bromazepam, chlordiazepoxide, clonazepam, clorazepate, diazepam, flurazepam, lorazepam, oxazepam, temazepam, triazolam) or antihistamines (e.g. hydroxyzine). In one embodiment, the anxiolytic is selected from the group consisting of alprazolam, bromazepam, chlordiazepoxide, clonazepam, clorazepate, diazepam, flurazepam, lorazepam, oxazepam, temazepam, triazolam, and hydroxyzine; or salts thereof.

The term “behavioral therapy”, as used herein, refers to, but is not limited to, cognitive behavioral therapy, in particular cognitive behavioral therapy features such as checking unhelpful thoughts, analyzing depressive thinking, analyzing cognitive distortions, scheduling positive behaviors, developing helpful thoughts to improve depressive symptoms and coping with MS symptoms such as fatigue or brain fog. In one embodiment, term “behavioral therapy”, as used herein, refers to cognitive behavioral therapy focused on coping with symptoms of multiple sclerosis and mood management. In one embodiment, term “behavioral therapy”, as used herein, refers to cognitive behavioral therapy focused on coping with symptoms of multiple sclerosis or mood management. In a further embodiment, term “behavioral therapy”, as used herein, refers to cognitive behavioral therapy focused on coping with symptoms of multiple sclerosis. In another embodiment, term “behavioral therapy”, as used herein, refers to cognitive behavioral therapy focused on mood management. In one embodiment, the behavioral therapy is cognitive behavioral therapy for depressive disorder associated with multiple sclerosis, as defined herein. In another embodiment, the behavioral therapy is cognitive behavioral therapy for depression associated with multiple sclerosis, as defined herein.

The term “cognitive behavioral therapy for depression associated with multiple sclerosis”, as used herein, refers to cognitive and behavioral techniques for the treatment of depressive symptoms in patients with MS, such as cognitive behavioral techniques that support management of depressive symptoms in patients with MS [e.g. Kidd T., et al., in PLOS ONE, 2017, 1-16; Hind D., et al., in BMC psychiatry, 2014, 14(1):5].

The term “cognitive behavioral therapy for depressive disorder associated with multiple sclerosis”, as used herein, refers to cognitive and behavioral techniques for the treatment of depressive disorder (e.g. major depressive disorder) in patients with MS [e.g. Mohr D., et al., in J Consult Clin Psychol, 2001, Vol. 69, No. 6, 942-949].

The term “computer-implemented device”, as used herein, refers to a device that can provide computerized therapy, such as computerized behavioral therapy, in particular computerized cognitive behavioral therapy.

The term “computer-implemented” in the expression “computer-implemented behavioral therapy”, as used herein, refers to behavioral therapy implemented by the use of electronic tools such as an online tool, a mobile device or an App.

The term “computer-implemented mobile device”, as used herein, refers to a portable device that can provide computerized therapy, such as computerized behavioral therapy, in particular computerized cognitive behavioral therapy.

The term “on demand”, as used herein, refers to the ability to allow a user (e.g. a patient) to initiate a computer-implemented device (e.g. a computerized behavioral therapy device), or digital therapeutic, at any desired time.

The term “system-prompted”, as used herein, refers to the ability, for example, of a computer-implemented device (e.g. computerized behavioral therapy device) to prompt a user (e.g. patient) to initiate the therapy provided by such a device at any time, for example once a day.

The term “mobile device”, as used herein, refers to a small, handheld computing device that can run applications software. Examples, not limited to, include smartphones and mobile computers (e.g. tablet computer, wearable computer).

The term “digital-therapeutic”, as used herein, is to be understood in the context of “software as a medical device” (SaMD), for example, as defined by the International Medical Device Regulation Forum (i.e. IMDRF SaMD Working Group in “Software as a Medical Device (SaMD): Key Definitions, 9 Dec. 2013). In one embodiment, SaMD refers to software intended to be used for one or more medical purposes that perform these purpose without being part of a hardware medical device (i.e. its intended purpose is not to drive a hardware medical device). In one embodiment, SaMD provides means and suggestions for mitigation of a disease. In a further embodiment, SaMD, as used herein, refers to software intended to be used for one or more medical purposes, that perform these purpose without being part of a hardware medical device (i.e. without being part of a medical device intended for dedicated medical use, but instead being capable of running on a general purpose computing device), and it provides means and suggestions for mitigation of a disease (i.e. provides therapy tools, for example psychosocial tools, such as behavioral therapy, for example cognitive behavioral therapy, for example, by providing skill building to aid treatment e.g. to aid copying with disease's symptoms or prevent relapse). The term “digital-therapeutics application”, as used herein, refers to a software as a medical device (i.e. a digital-therapeutic), as defined herein. In one embodiment, the term “digital-therapeutic”, as used herein, refers to digiceutical and thus both terms are interchangeable herein above and below.

The term “computerized behavioral therapy”, as used herein, refers to the delivery of behavioral therapy, such as cognitive behavioral therapy (CBT), via an interactive computer interface (e.g. delivered by a smartphone) instead of a face-to-face with a human therapist, namely the computer-based delivery of CBT without therapist.

The term “mood management”, as used herein, refers to building skills to effectively manage difficult emotions such as, for example, anger (i.e. anger management), anxiety (i.e. anxiety management) or depression.

The term “coping with symptoms of multiple sclerosis”, as used herein, refers to strategies, such as behavioral efforts, to manage symptoms of multiple sclerosis.

The term “symptoms of multiple sclerosis”, as used herein, refers to one or more (i.e. at least one) of symptoms associated with multiple sclerosis, for example, fatigue, brain fog, chronic pain, motor paralysis or clumsiness.

The term “treat” “treating” “treatment” or “therapy”, as used herein, means obtaining beneficial or desired results, for example, clinical results. Beneficial or desired results can include, but are not limited to, alleviation of one or more symptoms. One aspect of the treatment is, for example, that said treatment should have a minimal adverse effect on the patient, e.g. it should have a high level of safety. The term “alleviation”, for example in reference to a symptom of a condition, as used herein, refers to reducing at least one of the frequency and amplitude of a symptom of a condition in a patient. In one embodiment, the term “method for the treatment” or “method for a treatment”, as used herein, refers to “method to treat”.

The term “a therapeutically effective amount” or “an effective amount” of a compound of the present disclosure refers to an amount of a compound of the present disclosure that will elicit the biological or medical response of a subject, for example, ameliorate symptoms, alleviate conditions, slow or delay disease progression, etc. In another embodiment, the term refers to the amount of the compound of the present disclosure that, when administered to a subject, is effective to at least partially alleviate and/or ameliorate a condition, or a disorder or a disease.

The term “concomitant”, as used herein, refers to both simultaneous and sequential administration.

As used herein, the term “subject” refers to a mammalian organism, preferably a human being (male or female).

As used herein, the term “patient” refers to a subject who is diseased and would benefit from the treatment.

As used herein, a subject is “in need of” a treatment if such subject (patient) would benefit biologically, medically or in quality of life from such treatment.

The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.

The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein. In particular, reference to, for example, disease-modifying therapy for multiple sclerosis in combination with a further active agent, as used herein (e.g. in any of embodiments herein above, or in any of the claims, herein below), refers to a disease-modifying therapy for multiple sclerosis (e.g. in free form or in a pharmaceutical salt form) in combination with at least one further active agent, for example selected from the group consisting of a disease-modifying therapy for multiple sclerosis, (e.g. in free form or in a pharmaceutical salt form) and an anxiolytic (e.g. in free form or in a pharmaceutical salt form) or an antidepressant anxiolytic (e.g. in free form or in a pharmaceutical salt form).

The term “combination”, as used herein, in relation to an active ingredient (e.g. a disease-modifying therapy for multiple sclerosis) and a computer-implemented device or a software as medical device (i.e. digital-therapeutic), refers to a non-fixed combination or a kit of parts for the combined treatment, wherein the active ingredient [e.g. a disease-modifying therapy for multiple sclerosis, and optionally one or more drug partner (e.g. another drug as specified herein, also referred to as further “pharmaceutical active ingredient”, “therapeutic agent” or “co-agent”, such as an antidepressant or an anxiolytic) as a “fixed pharmaceutical combination” or as a “non-fixed pharmaceutical combination”], may be administered independently at the same time or separately within time intervals, especially where these time intervals allow a cooperative effect, e.g. synergistic effect. The terms “co-administration” or “combined administration” or the like as utilized herein are meant to encompass administration of the selected combination to a single subject in need thereof (e.g. a patient).

The term “pharmaceutical combination” refers to either a “fixed pharmaceutical combination” in one unit dosage form (e.g., capsule, tablet), a “non-fixed pharmaceutical combination”, or a kit of parts for the combined administration where the disease-modifying therapy for multiple sclerosis and one or more drug partner (e.g. another drug as specified herein, also referred to as further “pharmaceutical active ingredient”, “therapeutic agent”, “combination partner” or “co-agent”) may be administered independently at the same time or separately within time intervals. The term “fixed pharmaceutical combination” means that the active ingredients, e.g. the disease-modifying therapy for multiple sclerosis and one or more combination partners, are both administered to a patient simultaneously in the form of a single entity or dosage. The term “non-fixed pharmaceutical combination” means that the active ingredients, e.g. disease-modifying therapy for multiple sclerosis sand one or more combination partners, are both administered to a patient as separate entities either simultaneously or sequentially with no specific time limits, wherein such administration provides therapeutically effective levels of the two compounds in the body of the patient.

As used herein, the terms “free form” or “free forms” refers to the compound in non-salt form, such as the base free form or the acid free form of a respective compound, e.g. the compounds specified herein.

As used herein, the terms “salt”, “salts” or “salt form” refers to an acid addition or base addition salt of a respective compound, e.g. the compounds specified herein. “Salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein, may be capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

The compounds specified herein can be administered by conventional route.

The pharmaceutical composition or combination of the present disclosure can be in a unit dosage form (e.g. tablet), wherein the appropriate dosage of the active ingredient may vary depending upon a variety of factors, such as, for example, the age, weight, sex, the route of administration or salt employed.

### EXAMPLES

The following Examples serve to illustrate the disclosure without limiting the scope thereof.

### Example 1: The Digital-Therapeutics Application and User Interfaces

Some implementations of the disclosed technology will be described more fully with reference to the accompanying drawings. This disclosed technology may, however, be embodied in many different forms and should not be construed as limited to the implementations set forth herein.

Example implementations of the disclosed technology provide electronic devices, methods, and digital therapeutics for treating depressive symptoms associated with multiple sclerosis.

Example implementations of the disclosed technology will now be described with reference to the accompanying figures.

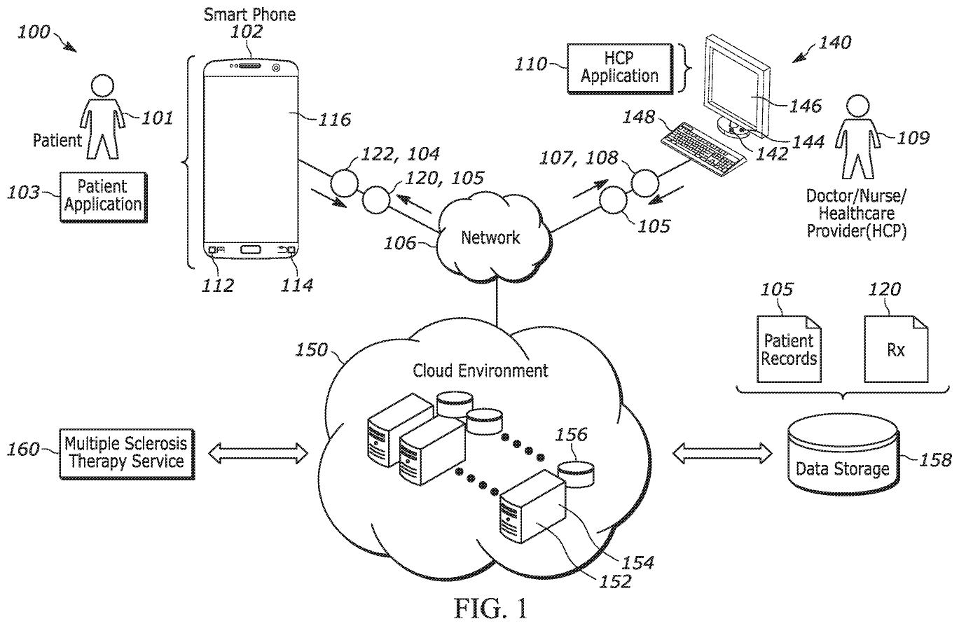

Referring to FIG. 1, in some implementations, a therapy prescription system 100 provides a patient 101 access to a prescription digital therapeutic 120 prescribed to the patient 101 and monitors events associated with the patient's 101 interaction with the prescription digital therapeutic 120. Although the digital therapeutic 120 is described herein as being a “prescription” digital therapeutic, it is understood that, according to some implementations, the digital therapeutic 120 will not require a prescription from a clinician. Rather, in such implementations, the digital therapeutic 120 may be available to a patient without a prescription, and the digital therapeutic 120 nonetheless otherwise functions in accordance with the description of the prescription digital therapeutic 120 described herein. According to implementations in which the digital therapeutic 120 is not prescribed, the person using or being administered the digital therapeutic may be referred to as a “user.” A “user” may include a patient 101 or any other person using or being administered the digital therapeutic 120, irrespective of whether the digital therapeutic 120 was prescribed to that person.

As used herein, a digital therapy may also be referred to as a digital-therapeutic configured to deliver evidence-based psychosocial intervention techniques for treating a patient with a particular disease or disorder, as well as symptoms and/or behaviors associated with the particular disease or disorder. In the instant case, the patient 101 is diagnosed with multiple sclerosis (MS) and the prescription digital therapeutic 120 is specifically tailored for addressing one or more depressive symptoms associated with MS that the patient 101 may experience. An authorized healthcare provider (HCP) 109 (e.g., a doctor, nurse, etc.) supervising the patient 101 diagnosed with MS may prescribe the patient 101 the prescription digital therapeutic 120 designed to help the patient 101 identify feelings the patient 101 is experiencing and modify dysfunction emotions, behaviors, and thoughts in order to treat depressive symptoms in the patient 101. The HCP 109 may include a physician, nurse, clinician, or other health professional qualified for treating patients diagnosed with multiple sclerosis (“MS”).

In some examples, the system 100 includes a network 106, a patient device 102, an HCP system 140, and a multiple sclerosis therapy service 160. The network 106 provides access to cloud computing resources 150 (e.g., distributed system) that execute the multiple sclerosis therapy service 160 to provide for the performance of services on remote devices. Accordingly, the network 106 allows for interaction between patients 101 and HCPs 109 with the multiple sclerosis therapy service 160. For instance, the multiple sclerosis therapy service 160 may provide the patient 101 access to the prescription digital therapeutic 120 and receive event data 122 inputted by the patient 101 associated with the patient's 101 interaction with the prescription digital therapeutic 120. In turn, the multiple sclerosis therapy service 160 may store the event data 122 on a storage resource 156.

The network 106 may include any type of network that allows sending and receiving communication signals, such as a wireless telecommunication network, a cellular telephone network, a time division multiple access (TDMA) network, a code division multiple access (CDMA) network, Global system for mobile communications (GSM), a third generation (3G) network, fourth generation (4G) network, a satellite communications network, and other communication networks. The network 106 may include one or more of a Wide Area Network (WAN), a Local Area Network (LAN), and a Personal Area Network (PAN). In some examples, the network 106 includes a combination of data networks, telecommunication networks, and a combination of data and telecommunication networks. The patient device 102, the HCP system 140, and the multiple sclerosis therapy service 160 communicate with each other by sending and receiving signals (wired or wireless) via the network 106. In some examples, the network 106 provides access to cloud computing resources, which may be elastic/on-demand computing and/or storage resources 156 available over the network 106. The term ‘cloud’ services generally refers to a service performed not locally on a user's device, but rather delivered from one or more remote devices accessible via one or more networks 106.

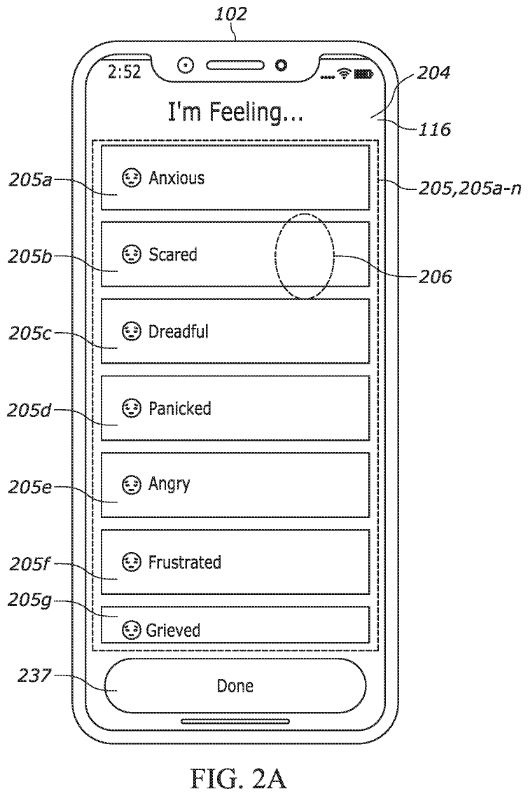

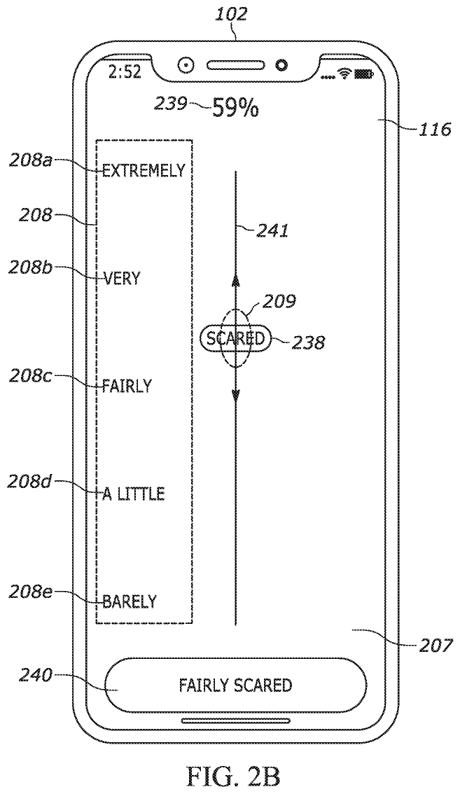

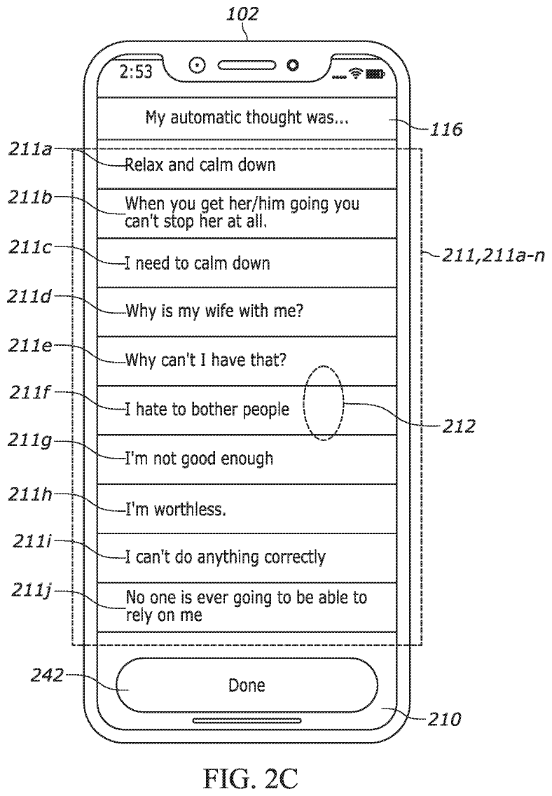

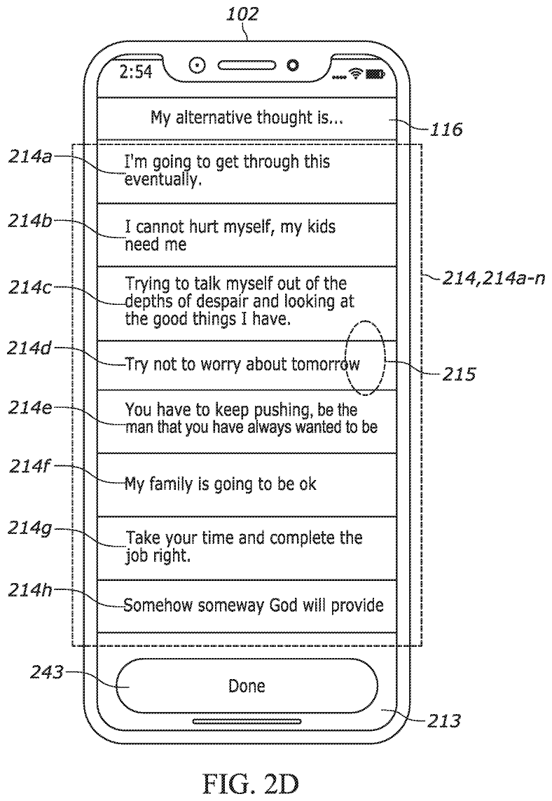

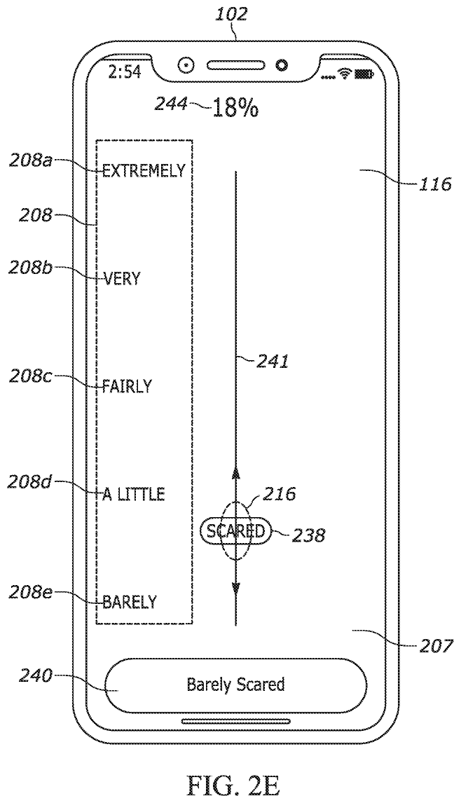

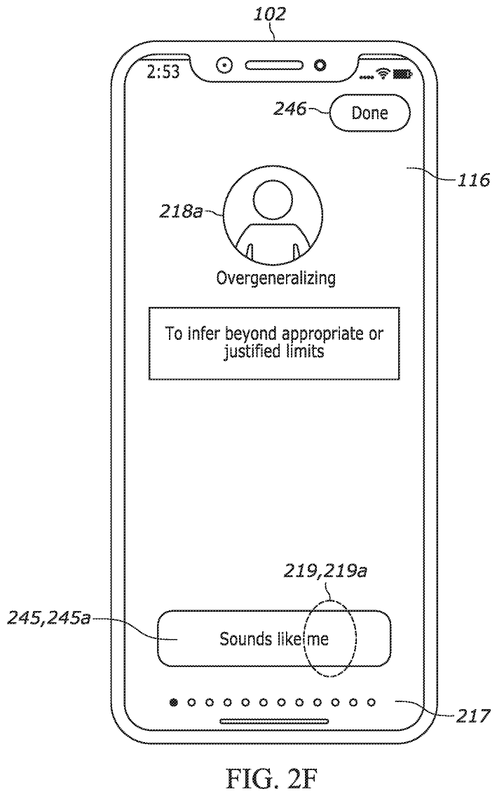

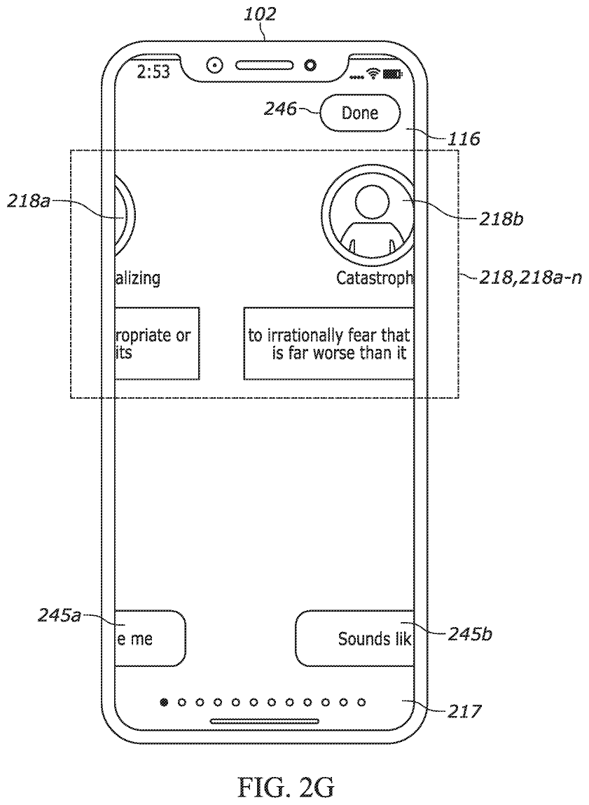

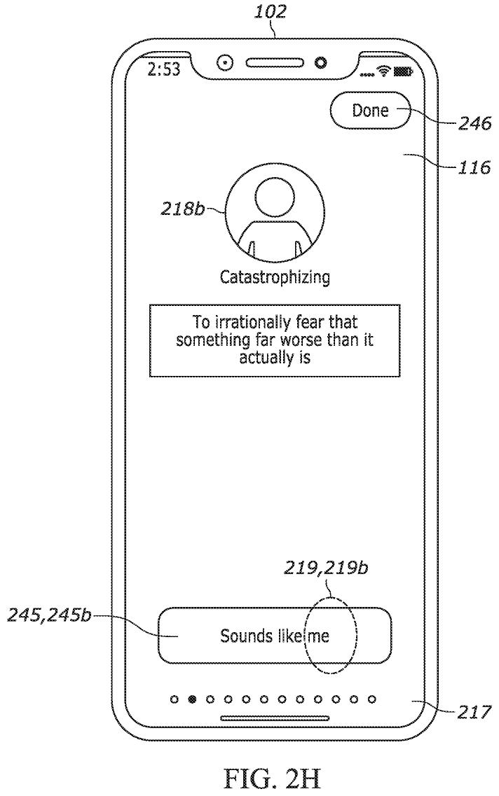

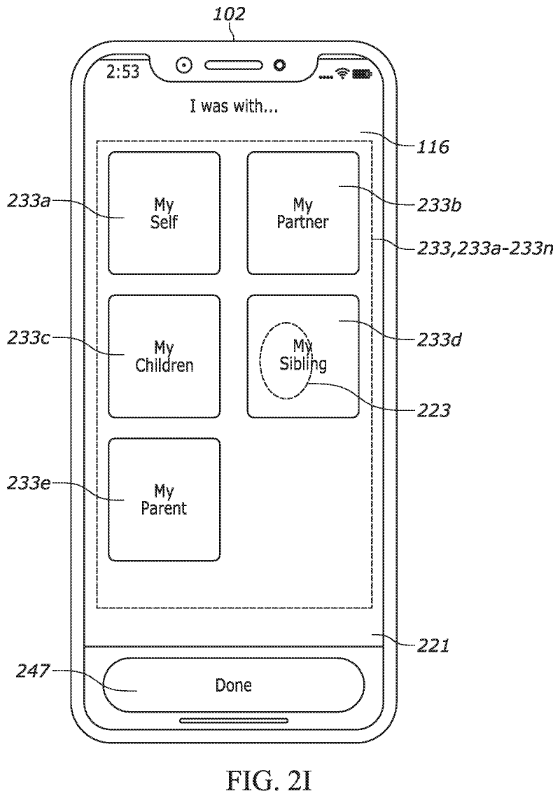

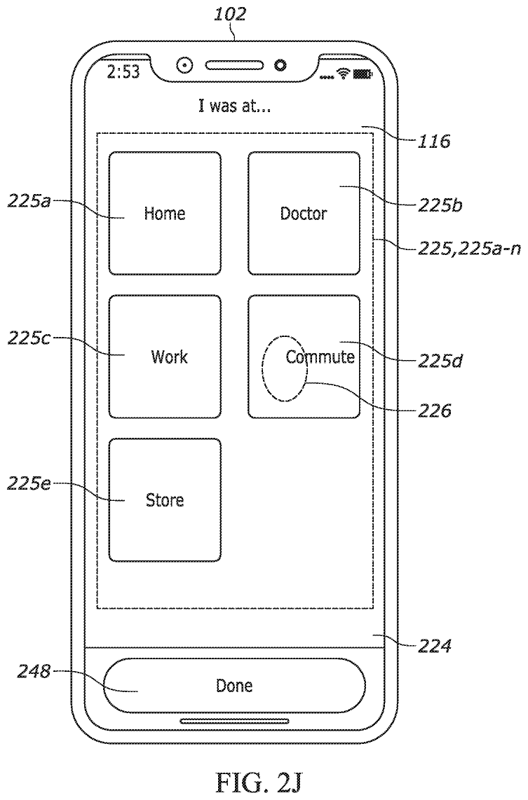

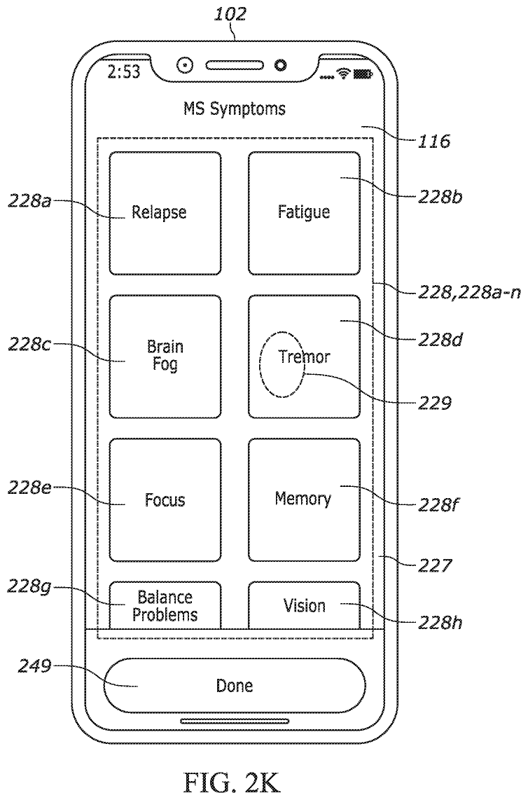

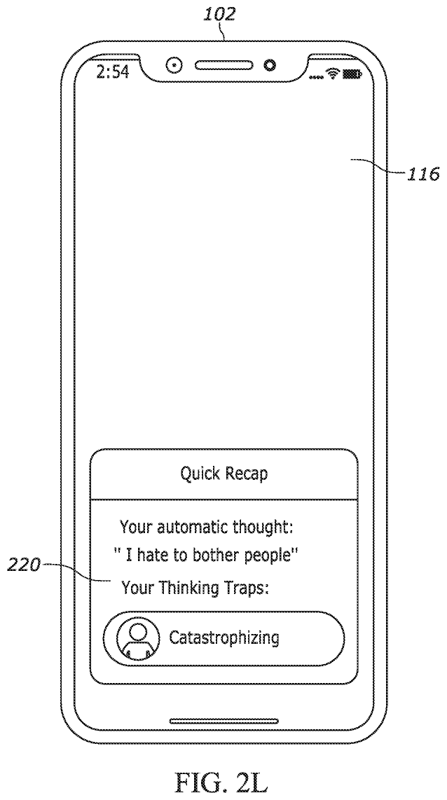

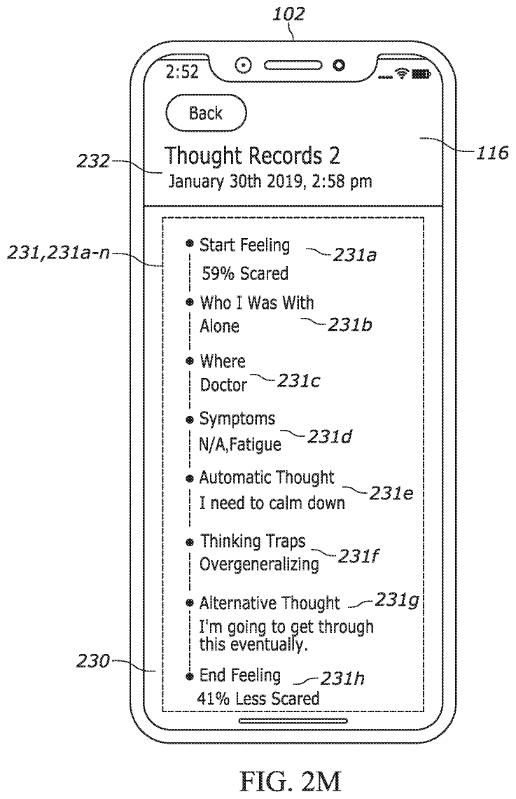

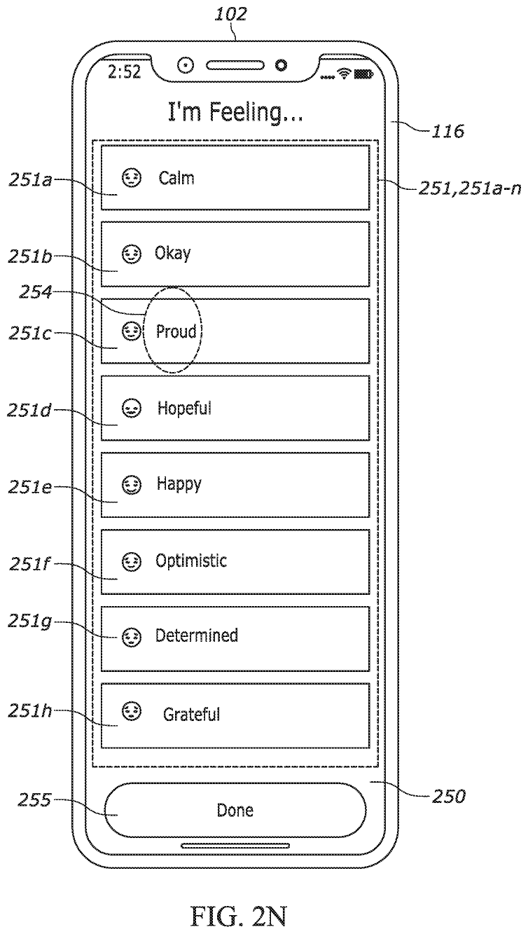

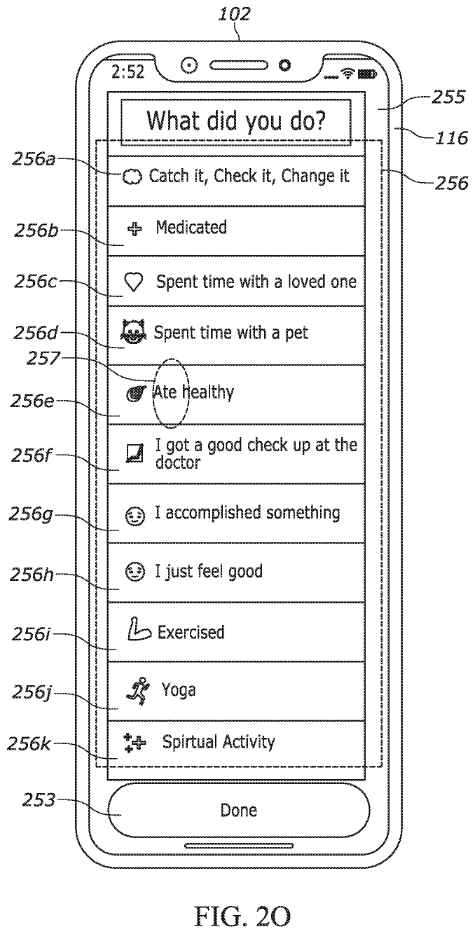

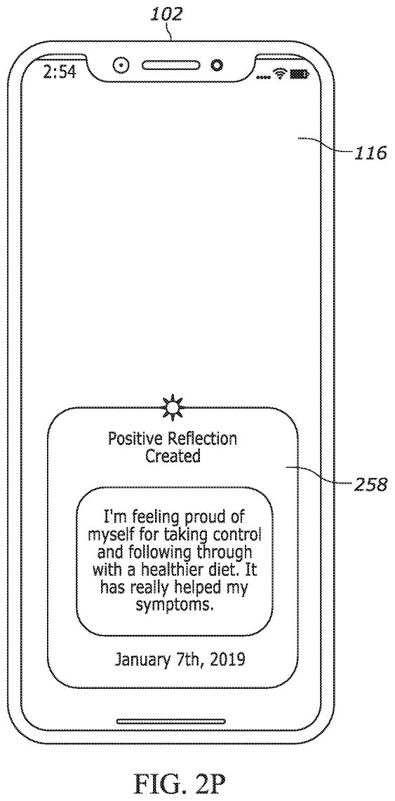

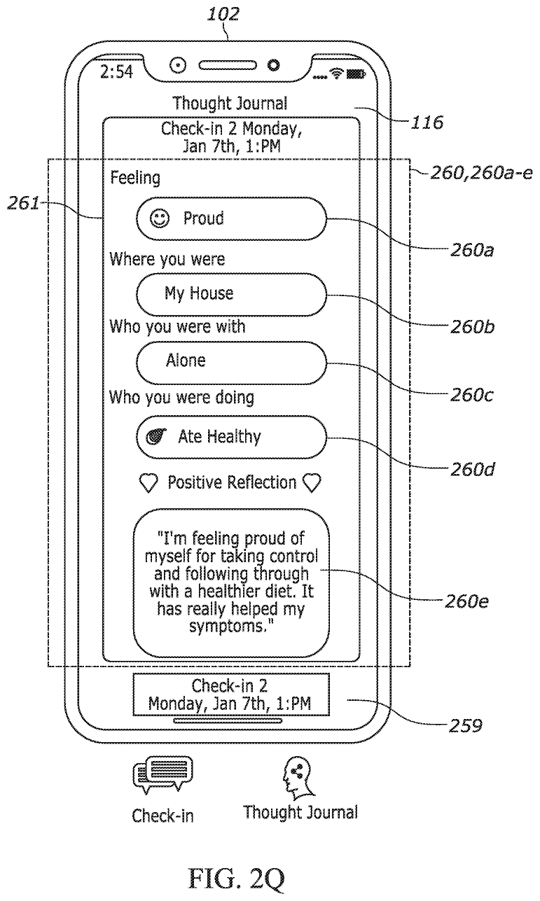

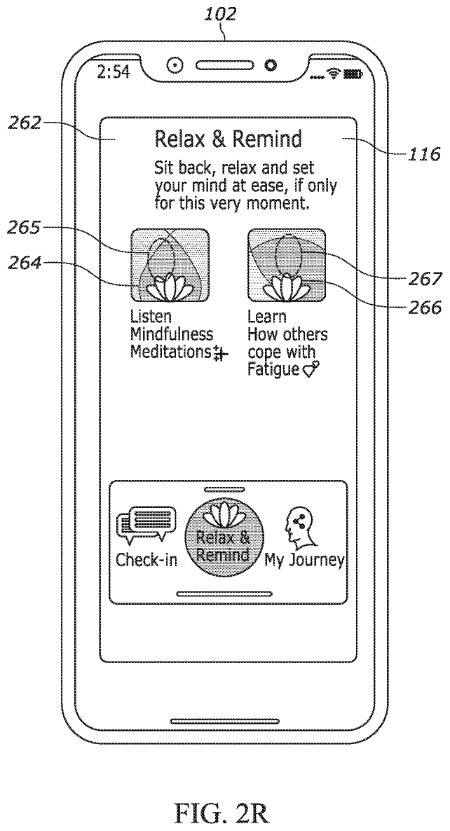

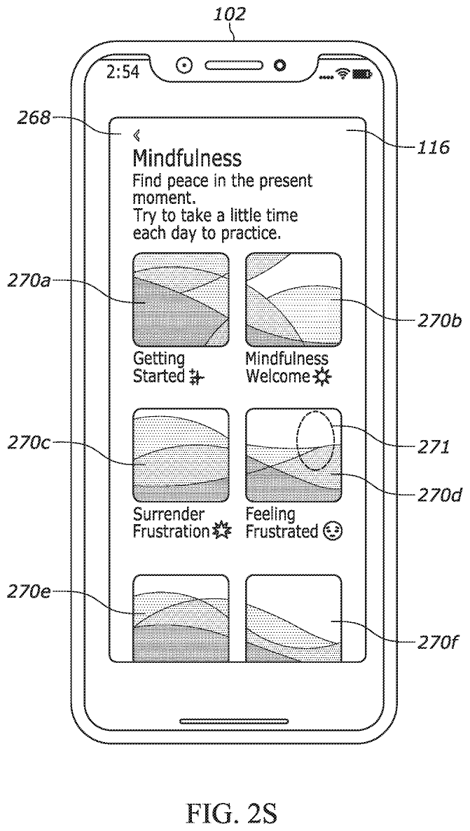

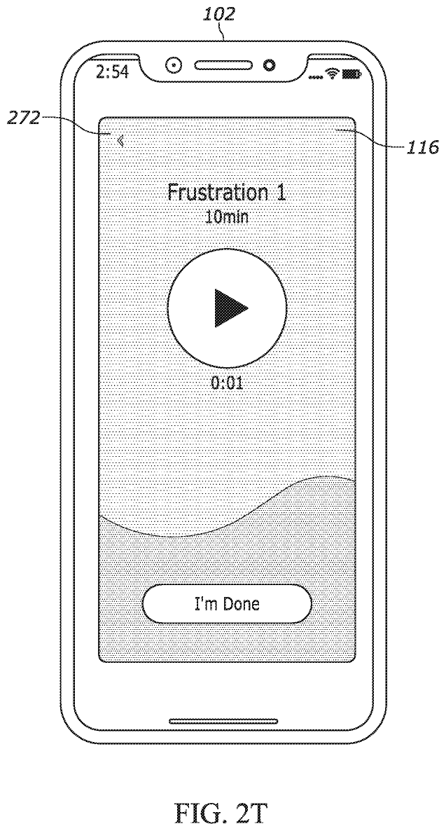

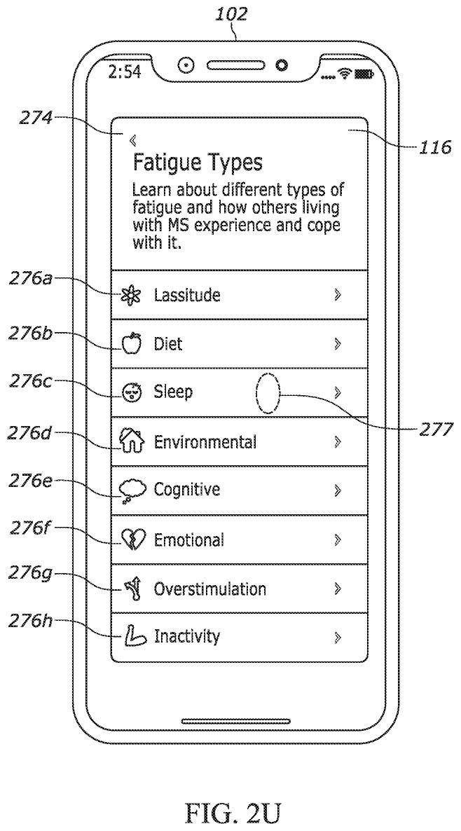

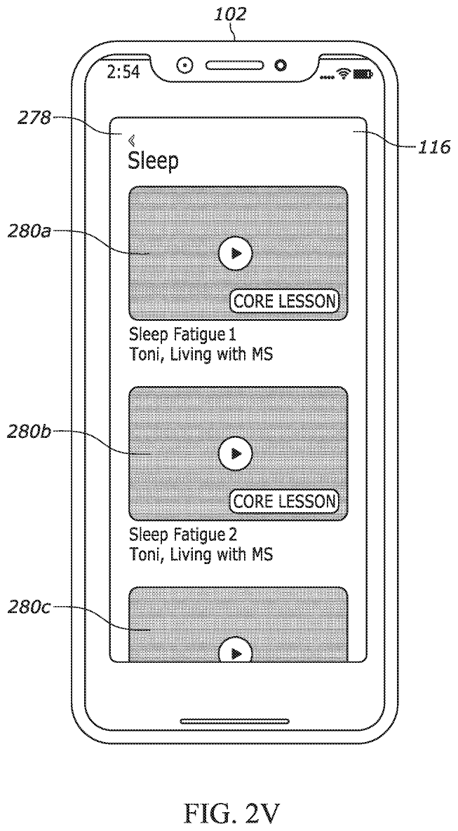

The patient device 102 may include, but is not limited to, a portable electronic device (e.g., smartphone, cellular phone, personal digital assistant, personal computer, or wireless tablet device), a desktop computer, or any other electronic device capable of sending and receiving information via the network 106. The patient device 102 includes data processing hardware 112 (a computing device that executes instructions), memory hardware 114, and a display 116 in communication with the data processing hardware 112. In some examples, the patient device 102 includes a keyboard 148, mouse, microphones, and/or a camera for allowing the patient 101 to input data. In addition to or in lieu of the display 116, the patient device 102 may include one or more speakers to output audio data to the patient 101. For instance, audible alerts may be output by the speaker to notify the patient 101 about some time sensitive event associated with the prescription digital therapeutic 120. In some implementations, the patient device 102 executes a patient application 103 (or accesses a web-based patient application) for establishing a connection with the multiple sclerosis therapy service 160 to access the prescription digital therapeutic 120. For instance, the patient 101 may have access to the patient application 103 for a duration (e.g., 3 months) of the prescription digital therapeutic 120 prescribed to the patient 101. Here, the patient device 102 may launch the patient application 103 by initially providing an access code 104 when the prescription digital therapeutic 120 is prescribed by the HCP 109 that allows the patient 101 to access content associated with the prescription digital therapeutic 120 from the multiple sclerosis therapy service 160 that is specifically tailored for treating/addressing one or more symptoms associated with MS that the patient 101 may be experiencing. The patient application 103, when executing on the data processing hardware 112 of the patient device 102, is configured to display a variety of graphical user interfaces (GUIs) (e.g., the feeling selection GUI 204 shown at FIG. 2A) on the display 116 of the patient device 102 that, among other things, allow the patient 101 to input event data 122 associated particular feelings the patient is experiencing, solicit information from the patient 101, and present journal entries for the patient 101 to view. The GUIs operate generally to guide the patient 101 through the use of the patient application 103, requiring an operator input to progress through the variety of GUIs in a continued and guided manner, as will become more apparent from the following.

The patient application 120 may send notifications to the patient device 102. In some embodiments, the patient application 120 may send notifications to the patient device 102 even when the application is not running on the patient device. The notifications may be sent to the notification center of the patient device 102. The notifications may remind the patient 101, daily, weekly, or otherwise periodically to run and engage with the patient application 103. For example, the patient application 120 may cause a notification to be sent to the patient device 102 every evening to remind the patient 101 to open the patient application 102. The notifications may require explicit acknowledgement by the patient 101, for example by the patient 101 operating the patient device 102 and opening the patient application 103 to acknowledge the notification, for example by pressing an appropriate button or operating another GUI feature on the patient application 103.

The storage resources 156 may provide data storage 158 for storing the event data 122 received from the patient 101 in a corresponding patient record 105 as well as the prescription digital therapeutic 120 prescribed to the patient 101. The patient record 105 may be encrypted while stored on in the data storage 158 so that any information identifying patient 101 is anonymized, but may later be de-crypted when the patient 101 or supervising HCP 109 requests the patient record 105 (assuming the requester is authorized/authenticated to access the patient record 105). All data transmitted over the network 106 between the patient device 102 and the cloud computing system 150 may be encrypted and sent over secure communication channels. For instance, the patient application 103 may encrypt the event data 122 before transmitting to the multiple sclerosis therapy service 160 via the HTTPS protocol and decrypt a patient record 105 received from the multiple sclerosis therapy service 160. When network connectivity is not available, the patient application 103 may store the event data 122 in an encrypted queue within the memory hardware 114 until network connectivity is available.

The HCP system 140 may be located at a clinic, doctor's office, or facility administered by the HCP 109 and includes data processing hardware 142, memory hardware 144, and a display 146. The memory hardware 144 and the display 146 are in communication with the data processing hardware 142. For instance, the data processing hardware 142 may reside on a desktop computer or portable electronic device for allowing the HCP 109 to input and retrieve data to and from the multiple sclerosis therapy service 160. In some examples, the HCP 109 may initially onboard some or all of patient data 107 at the time of prescribing the prescription digital therapeutic 120 to the patient 101. The HCP system 140 includes a keyboard 148, mouse, microphones, speakers and/or a camera. In some implementations, the HCP system 140 (i.e., via the data processing hardware 142) executes a HCP application 110 (or accesses a web-based patient application) for establishing a connection with the multiple sclerosis therapy service 160 to input and retrieve data therefrom. For instance, the HCP system 140 may be able to access the anonymized patient record 105 securely stored by the multiple sclerosis therapy service 160 on the storage resources 156 by providing an authentication token 108 validating that the HCP 109 is supervising the patient 101 and authorized to access the corresponding patient record 105. The authentication token 108 may identify the particular patient 101 associated with the patient record 105 that the HCP system 140 is permitted to obtain from the multiple sclerosis therapy service 160. The patient record 105 may include time-stamped event data 122 indicating the patient's interaction with the prescription digital therapeutic 120 through the patient application 103 executing on the patient device 102.

The cloud computing resources 150 may be a distributed system (e.g., remote environment) having scalable/elastic resources 152. The resources 152 include computing resources 154 (e.g., data processing hardware) and/or the storage resources 156 (e.g., memory hardware). The cloud computing resources 150 execute the multiple sclerosis therapy service 160 for facilitating communications with the patient device 102 and the HCP system 140 and storing data on the storage resources 156 within the data storage 158. In some examples, multiple sclerosis therapy service 160 and the data storage 158 reside on a standalone computing device. The multiple sclerosis therapy service 160 may provide the patient 101 with the patient application 103 (e.g., a mobile application, a web-site application, or a downloadable program that includes a set of instructions) executable on the data processing hardware 112 and accessible through the network 106 via the patient device 102 when the patient 101 provides a valid access code 104. Similarly, the multiple sclerosis therapy service 160 may provide the HCP 109 with the HCP application 110 (e.g., a mobile application, a web-site application, or a downloadable program that includes a set of instructions) executable on the data processing hardware 142 and accessible through the network 106 via the HCP system 140.

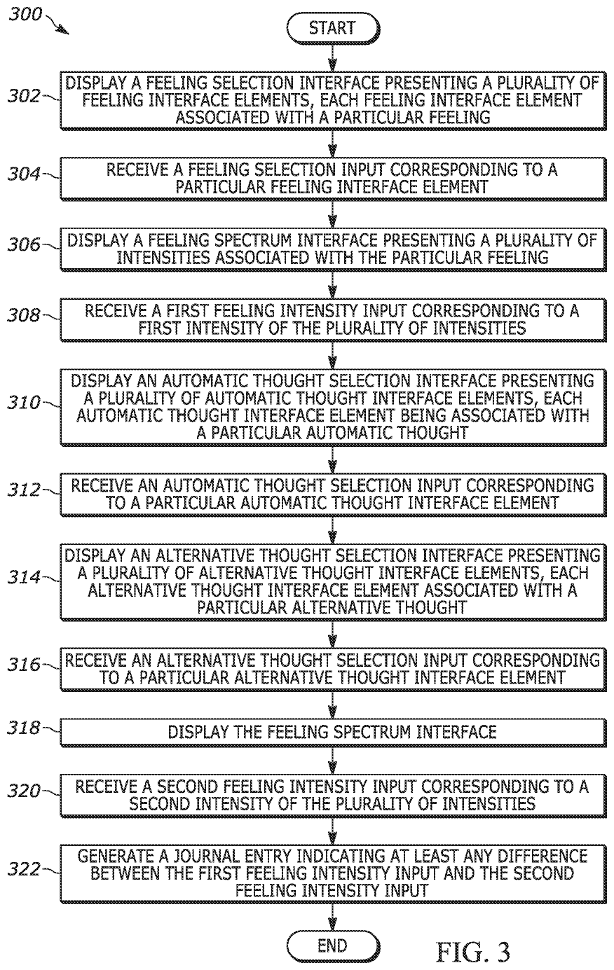

FIGS. 2A-2Q illustrate schematic views of exemplary GUIs of the prescription digital therapeutic 120 (e.g., by execution of the patient application 103) displayed on the display 116 of the patient device 102 for treating depressive symptoms associated with MS. The example GUIs are configured to display graphical elements (e.g., buttons) that the patient 101 may select via user inputs such as touch inputs, speech inputs, or other input techniques such as via a mouse, stylus, keyboard, gesture, or eye gaze. Generally, and as will be apparent in more detail from the following description, and from the flow diagram in FIG. 3, the GUIs are interactive user interfaces in that they present to the user user-selectable graphical elements that allow the user to interact with, select items on, or input information into the patient application 103, and the patient application 103—representative of the digital therapeutic 120—to then respond by the presentation to the user of a next, different, GUI selected to be displayed as part of the digital therapeutic 120 in dependence on the input into, selection, or interaction with the previously-displayed GUI by the user. This two-way man-machine interaction process of GUI display, user selection, data input, or interaction with the displayed GUI, and then display of a next, different, GUI in response to the user selection, input, or interaction with the previously displayed GUI continues until the operation of the digital therapeutic 120 is complete. Such operation therefore provides a continued and guided patient/user-machine interaction to provide the therapeutic effect.

In addition, the patient application 103 also provides a patient use logging tool, which keeps track of the patient's 101 interactions with the patient device 102 whilst using the digital therapeutic 120, data representative of the interactions is stored, including a time-stamp, and may be collated and sent to the multiple sclerosis therapy service 160. Such a patient usage logging tool can assist in tracking the progress and efficacy of the digital therapeutic 120 in treating the patient 101.

Referring to FIG. 2A, in some implementations, upon launching the patient application 103 associated with the prescription digital therapeutic 120 prescribed to the patient 101, the patient application 103 displays a feeling selection GUI 204 that allows the patient 101 to input a particular feeling they are presently experiencing, or has recently experienced. In the example shown, the feeling selection GUI 204 provides a plurality of feeling interface elements 205, each 205a-n associated with a corresponding feeling the patient 101 is experiencing or has recently experienced. While the example shown depicts interface elements 205a-205g, the patient 101 may view additional interface elements 205n by scrolling (e.g., via a swipe gesture). The plurality of feeling interface elements 205 may be prepopulated based on common feelings a typical patient diagnosed with MS may be experiencing. The patient 101 may indicate their current feelings by selecting the corresponding feeling interface element 205 displayed in the feeling selection GUI 204. In the example shown, a first feeling interface element 205a (“Anxious”) indicates that the patient 101 is feeling anxious, a second feeling interface element 205b (“Scared”) indicates that the patient 101 is feeling scared, a third feeling interface element 205c (“Dreadful”) indicates that the patient 101 is feeling dreadful, a fourth feeling interface element 205d (“Panicked”) indicates that the patient 101 is feeling panicked, a fifth feeling interface element 205e (“Angry”) indicates that the patient 101 is feeling angry, a sixth feeling interface element 205f (“Frustrated”) indicates that the patient 101 is feeling frustrated, and a seventh feeling interface element 205g (“Grieved”) indicates that the patient 101 is feeling grieved.

The feeling interface elements 205a-205g do not represent an exhaustive list of all feeling interface elements, but rather an exemplary list of feeling interface elements that may be included as part of the feeling selection GUI 204. Furthermore, the feeling selection GUI 204 may include other feeling interface elements in addition to feeling interface elements 205a-205g, or may omit one or more of feeling interface elements 205a-205g, without departing from the teachings herein. In some implementations, each of the plurality of feeling interface elements 205 is categorized as being associated with one of “Negative” feelings or “Positive” feelings, such that additional feeling interface elements 205 within the Positive category (e.g., FIG. 2N) may be associated with feelings such as calm (“Calm”), neutral (“Okay”), prideful (“Proud”), optimistic (“Hopeful”), or content (“Happy”).

In the example shown, the patient device 102 detects a first sequence of inputs, the first sequence of inputs including a feeling selection input 206 (e.g., touch or spoken) corresponding to the feeling element interface 205b (“Scared”) indicating they are feeling scared. As used herein, a sequence of inputs can be a single input. In some implementations, the feeling selection input 206 causes the patient application 103 to transmit time-stamped event data 122 to the multiple sclerosis therapy service 160 (FIG. 1) that includes a selection indication indicating that the patient is presently feeling scared. By sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the feeling selection input 206 causes the patient application 103 to generate, for display on the patient device 102, a journal interface element of the plurality of journal interface elements 231 (FIG. 2M), the journal interface element indicating the selected feeling. In other examples, the feeling selection input 206 causes the patient application 103 to modify the already-generated plurality of journal interface elements 231 to indicate the selected feeling.

After detecting selection of a feeling interface element 205, the patient application 103 advances to display a feeling spectrum GUI 207 (FIG. 2B) on the display 116 of the patient device 102. In some configurations, the feeling selection input 206 selecting the feeling interface element 205 causes the patient application 103 to automatically display the feeling spectrum GUI 207. In other configurations, the patient application 103 requires the patient 101 to first confirm the selected feeling interface element 205 by selecting a Feeling Selection Done Button 237 (e.g., as shown in FIG. 2A). In these configurations, the patient application 103 displays the feeling spectrum GUI 207 in response to a selection indication indicating selection of the Feeling Selection Done Button 237.

At FIG. 2B, in some configurations, the patient application 103 causes the patient device 102 to display the feeling spectrum GUI 207 that allows the patient 101 to input a feeling intensity of the particular feeling that the they are presently experiencing. In the example shown, the feeling spectrum GUI 207 provides a plurality of intensities 208, each individual intensity 208a-208e being associated with a corresponding intensity of the particular feeling the patient 101 may be presently experiencing. The patient 101 may indicate the present intensity of their current feelings by moving a Slider button 238 to select a corresponding intensity. In some configurations, Slider button 238 translates up and down a Scale 241, and the position of Slider button 238 relative to the Scale 241 indicates a particular intensity. For example, the location of the Slider button 238 relative to the Scale 241 is reflected in an intensity value 239. The intensity value 239 will provide the patient 101 with a numerical percentage value of their intensity of their current feeling. For example, if the patient 101 translates the Slider button 238 more than half way up the Scale 241, the intensity value 239 will reflect a higher percentage value. As seen in FIG. 2B, the location of the Slider button 238 relative to the Scale indicates the intensity of the feeling scared that the patient 101 is feeling, and the intensity value 239 indicates that the patient 101 is 59% scared.

With continued reference to FIG. 2B, in some configurations, the location of Slider button 238 relative to the Scale 241 will correspond to one of the plurality of intensities 208. The patient 101 may indicate a feeling intensity of the particular feeling that they are currently feeling by translating the Slider button 238 relative to the Scale 241 to correspond to one of the plurality of intensities 208 displayed in the feeling spectrum GUI 207. The plurality of intensities 208 correspond to the feeling selection input 206 that was selected in the prior GUI, feeling selection GUI 204. In the example shown, the plurality of intensities 208 correspond to the feeling of “scared”; a first intensity 208a (“Extremely”) indicates that the patient is feeling extremely scared, a second intensity 208b (“Very”) indicates that the patient is feeling very scared, a third intensity 208c (“Fairly”) indicates that the patient is feeling fairly scared, a fourth intensity 208d (“A little”) indicates that the patient is feeling a little scared, and the fifth intensity 208e (“Barely”) indicates that the patient is feeling barely scared. The intensities 208a-208e do not represent an exhaustive list of all intensities, but rather an exemplary list of feeling interface elements that may be included on the feeling spectrum GUI 207. Furthermore, feeling spectrum GUI 207 may include other intensities in addition to the intensities 208a-208e, or may omit one or more intensities 208a-208e.

In the example shown, the patient device 102 detects a second sequence of inputs, the second sequence of inputs including a first feeling intensity input 209 (e.g., touch or spoken) that selects the intensity 208c, corresponding to the intensity value 239, indicating that they are feeling fairly scared. In some implementations, the first feeling intensity input 209 causes the patient application 103 to transmit time-stamped event data 122 to the multiple sclerosis therapy service 160 (FIG. 1) that includes a selection indication indicating that the patient is presently feeling fairly scared. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the first feeling intensity input 209 causes the patient application 103 to generate, for display on the patient device 102, a journal interface element of the plurality of journal interface elements 231 (FIG. 2M), the journal interface element indicating the selected feeling intensity. In other examples, the first feeling intensity input 209 causes the patient application 103 to modify the already-generated plurality of journal interface elements 231 to indicate the selected feeling intensity.

After detecting selection of the plurality of intensities 208, the patient application 103 advances to display an automatic thought selection GUI 210 (FIG. 2C) on the display 116 of the patient device 102. In some configurations, the first feeling intensity input 209 selecting one of the plurality of intensities 208 causes the patient application 103 to automatically display the automatic thought selection GUI 210. In other configurations, the patient application 103 requires the patient 101 to first confirm the selected one of the plurality of intensities 208 by selecting a Feeling Spectrum Done Button 240 (e.g., as shown in FIG. 2B). In these configurations, the patient application 103 displays the automatic thought selection interface GUI 210 in response to a selection indication indicating selection of the Feeling Spectrum Done Button 240. According to some examples, and as shown in FIG. 2B, the text included within the Feeling Spectrum Done Button 240 may be based on the selected feeling intensity.

At FIG. 2C, in some configurations, the patient application 103 causes the patient device 102 to display the automatic thought selection GUI 210 that allows the patient 101 to input a particular automatic thought corresponding to their thoughts. In the example shown, the automatic thought selection GUI 210 provides a plurality of automatic thought interface elements 211, each individual automatic thought interface element 211a-211n being associated with a corresponding automatic thought that the patient 101 may have recently had, or currently has. While the example shown depicts automatic thought interface elements 211a-211j, the patient 101 may view additional interface elements 211n by scrolling (e.g., via a swipe gesture). The automatic thoughts represent thoughts that are common in patients with MS. As depicted in FIG. 2C, in the example shown, the particular thoughts are negative thoughts that users with MS experience that can cause depressive symptoms. Displaying common automatic thoughts advantageously allows the patient 101 to identify a particular thought that the patient has that may be associated with one or more depressive symptoms. The plurality of automatic thought interface elements 211 may be prepopulated based on common automatic thoughts a typical patient diagnosed with MS may have had or currently has. The patient 101 may indicate the automatic thought associated with them by selecting the corresponding automatic thought interface element 211 displayed in the automatic thought selection GUI 210. In the example shown, a first automatic thought interface element 211a (“Relax and calm down”) indicates that the patient 101 has or had the thought to relax and calm down, a second automatic thought interface element 211b (“When you get her/him going you can't stop her at all.”) indicates that the patient 101 has or had the thought that when you get him/her going you can't stop her at all, a third automatic thought interface element 211c (“I need to calm down”) indicates that the patient 101 has or had the thought that they need to calm down, a fourth automatic thought interface element 211d (“Why is my wife with me?”) indicates that the patient 101 has or had the thought asking why their wife is still with them, a fifth automatic thought interface element 211e (“Why can't I have that?”) indicates that the patient 101 has or had the thought asking why they can't have that, a sixth automatic thought interface element 211f (“I hate to bother people”) indicates that the patient 101 has or had the thought that they hate to bother people, a seventh automatic thought interface element 211g (“I'm not good enough”) indicates that the patient 101 has or had the thought that they are not good enough, an eighth automatic thought interface element 211h (“I'm worthless”) indicates that the patient 101 has or had the thought that they are worthless, a ninth automatic thought interface element 211i (“I can't do anything correctly”) indicates that the patient 101 has or had the thought that they can't do anything correctly, and a tenth automatic thought interface element 211j (“No one is ever going to be able to rely on me”) indicates that the patient 101 has or had the thought that no one is ever going to be able to rely on them.

The automatic thought interface elements 211a-211j do not represent an exhaustive list of all automatic thought interface elements, but rather an exemplary list of automatic thought interface elements that may be included on the automatic thought selection GUI 210. Furthermore, the automatic thought selection GUI 210 may include other automatic thought interface elements in addition to automatic thought interface elements 211a-211j, or may omit one or more automatic thought interface elements 211a-211j.

In the example shown, the patient device 102 detects a third sequence of inputs, the third sequence of inputs including an automatic thought selection input 212 (e.g., touch or spoken) corresponding to the automatic thought interface element 211f (“I hate to bother people”) indicating that the patient 101 has or has recently had the thought that they hate to bother people. In some implementations, the automatic thought selection input 212 causes the patient application 103 to transmit time-stamped event data 122 to the multiple sclerosis therapy service 160 (FIG. 1) that includes a selection indication indicating that the patient 101 has or had the thought that they hate to bother people. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the automatic thought selection input 212 causes the patient application 103 to generate, for display on the patient device 102, a journal interface element of the plurality of journal interface elements 231 (FIG. 2M), the journal interface element indicating the selected automatic thought. In other examples, the automatic thought selection input 212 causes the patient application 103 to modify the already-generated plurality of journal interface elements 231 to indicate the selected automatic thought.

After detecting selection of an automatic thought interface element 211, the patient application 103 advances to display an alternative thought selection GUI 213 (FIG. 2D) on the display 116 of the patient device 102. In some configurations, the automatic thought selection input 212 selecting the automatic thought interface element 211 causes the patient application 103 to automatically display the alternative thought selection GUI 213. In other configurations, the patient application 103 requires the patient 101 to first confirm the selected automatic thought interface element 211 by selecting an Automatic Thought Selection Done Button 242. In these configurations, the patient application 103 displays the alternative thought selection GUI 213 in response to a selection indication indicating selection of the Automatic Thought Selection Done Button 242.

At FIG. 2D, in some configurations, the patient application 103 causes the patient device 102 to display the alternative thought selection GUI 213 that allows a patient 101 to input a particular alternative thought corresponding to their thoughts. In the example shown, the alternative thought selection GUI 213 provides a plurality of alternative thought interface elements 214, each individual alternative thought interface element 214a-214n being associated with a corresponding alternative thought that the patient 101 can use to modify their thoughts and feelings. While the example shown depicts alternative thought interface elements 214a-214h, the patient 101 may view additional interface elements 214n by scrolling (e.g., via a swipe gesture). The alternative thoughts represent thoughts that can help users with MS modify their automatic thoughts by changing the distortion of their thoughts. The alternative thoughts reflect positive thoughts that patients with depressive symptoms associated with MS can think about to modify their automatic thought(s) that are related to their depressive symptoms. The plurality of alternative thought interface elements 214 may be prepopulated based on recommended alternative thoughts a typical patient diagnosed with MS would find beneficial to think about in order to modify automatic thought(s). The patient 101 may indicate the alternative thought that they would like to use to modify their feelings and thoughts by selecting the corresponding alternative thought interface element 214 displayed in the alternative thought selection GUI 213. In the example shown, a first alternative thought interface element 214a (“I'm going to get through this eventually”) indicates that the patient 101 would like to modify their thoughts to thinking that they are going to get through this eventually, a second alternative thought interface element 214b (“I cannot hurt myself, my kids need me”) indicates that the patient 101 would like to modify their thoughts to thinking that they cannot hurt themselves and their kids need them, a third alternative thought interface element 214c (“Trying to talk myself out of the depths of despair and looking at the good things I have”) indicates that the patient 101 would like to modify their thoughts to thinking to trying to talk himself or herself out of the depths of despair and to look at the good things they have, a fourth alternative thought interface element 214d (“Try not to worry about tomorrow”) indicates that the patient 101 would like to modify their thoughts to try not to worry about tomorrow, a fifth alternative thought interface element 214e (“You have to keep pushing, be the man that you have always wanted to be”) indicates that the patient 101 would like to modify their thoughts to thinking that they have to keep pushing to be the person that they have always wanted to be, a sixth alternative thought interface element 214f (“My family is going to be ok”) indicates that the patient 101 would like to modify their thoughts to thinking that their family is going to be ok, a seventh alternative thought interface element 214g (“Take your time and complete the job right”) indicates that the patient 101 would like to modify their thoughts to thinking about taking their time and to complete the job right, and an eighth alternative thought interface element 214h (“Somehow someway God will provide”) indicates that the patient 101 would like to modify their thoughts to thinking that somehow someway God will provide.

The alternative thought interface elements 214a-214h do not represent an exhaustive list of all alternative thought interface elements, but rather an exemplary list of alternative thought interface elements that may be included on the alternative thought selection GUI 213. Furthermore, the alternative thought selection GUI 213 may include other alternative thought interface elements in addition to alternative thought interface elements 214a-214h, or may omit one or more alternative thought interface elements 214a-214h.

In the example shown, the patient device 102 detects a fourth sequence of inputs, the fourth sequence of inputs including an alternative thought selection input 215 (e.g., touch or spoken) corresponding to the alternative thought interface element 214d (“Try not to worry about tomorrow”) indicating that the patient 101 would like to modify their thoughts to try not to worry about tomorrow. In some implementations, the alternative thought selection input 215 causes the patient application 103 to transmit time-stamped event data 122 to the multiple sclerosis therapy service 160 (FIG. 1) that includes a selection indication indicating that the patient would like to modify their thoughts to try not to worry about tomorrow. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the alternative thought selection input 215 causes the patient application 103 to generate, for display on the patient device 102, a journal interface element of the plurality of journal interface elements 231 (FIG. 2M), the journal interface element indicating the selected alternative thought. In other examples, the alternative thought selection input 215 causes the patient application 103 to modify the already-generated plurality of journal interface elements 231 to indicate the selected alternative thought.

After detecting selection of an alternative thought interface element 214, the patient application 103 advances to display the feeling spectrum GUI 207 (FIG. 2E) on the display 116 of the patient device 102. In some configurations, the alternative thought selection input 215 selecting the alternative thought interface element 214 causes the patient application 103 to automatically display the feeling spectrum GUI 207. In other configurations, the patient application 103 requires the patient 101 to first confirm the selected alternative thought interface element 214 by selecting an Alternative Thought Selection Done Button 243 (e.g., as shown in FIG. 2D). In these configurations, the patient application 103 displays the feeling spectrum GUI 207 in response to a selection indication indicating selection of the Alternative Thought Selection Done Button 243.

At FIG. 2E, in some configurations, the patient application 103 causes the patient device 102 to display again the feeling spectrum GUI 207 that allows a patient 101 to, again, input a feeling intensity of the particular feeling that they are presently experiencing or recently felt. In the example shown, the patient device 102 detects a fifth sequence of inputs, the fifth sequence of inputs including a second feeling intensity input 216 (e.g., touch or spoken) that selects the fifth intensity 208e, corresponding to an updated intensity value 244, indicating that they are feeling barely scared. In some implementations, the second feeling intensity input 216 causes the patient application 103 to transmit time-stamped event data 122 to the multiple sclerosis therapy service 160 (FIG. 1) that includes a selection indication indicating that the patient is presently feeling barely scared. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the second feeling intensity input 216 causes the patient application 103 to generate, for display on the patient device 102, a journal interface element of the plurality of journal interface elements 231 (FIG. 2M), the journal interface element indicating at least any difference between the first feeling intensity input 209 and the second feeling intensity input 216 (e.g., as reflected through a percentage decrease or the like). In other examples, the second feeling intensity input 216 causes the patient application 103 to modify the already-generated plurality of journal interface elements 231 to indicate at least any difference between the first feeling intensity input 209 and the second feeling intensity input 216.

After detecting selection of one of the plurality of intensities 208, the patient application 103 advances to display a next GUI on the display 116 of the patient device 102. In some configurations, the second feeling intensity input 216 selecting one of the plurality of intensities 208 causes the patient application 103 to automatically display the next GUI. In other configurations, the patient application 103 requires the patient 101 to first confirm the selected one of the plurality of intensities 208 by selecting the Feeling Spectrum Done Button 240. In these configurations, the patient application 103 displays the next GUI in response to a selection indication indicating selection of the Feeling Spectrum Done Button 240.

Referring now to FIGS. 2F-2M, the patient application 103 may display some or all of the GUIs corresponding to the figures. The GUIs corresponding to FIGS. 2F-2M may be displayed, if at all, in any particular order at any time the patient 101 interacts with the patient application 103.

At FIGS. 2F-2H, in some configurations, the patient application 103 causes the patient device to display a thinking traps GUI 217 that allows the patient 101 to input a thinking trap associated with the particular thoughts they are having. In the examples shown, the thinking traps GUI 217 provides a plurality of thinking trap interface elements 218, each individual think trap interface element 218a-218n being associated with a corresponding thinking trap the patient 101 may be presently thinking or has recently thought. It should be noted that while the example shown depicts the thinking traps GUI 217 displaying the plurality of thinking trap interface elements 218, in other examples, thinking traps GUI 217 can display any other type of cognitive distortions other than thinking traps. While the example shown depicts thinking trap interface elements 218a-218b, the patient 101 may view additional thinking trap interface elements 218n by scrolling (e.g., via a swipe gesture). The plurality of thinking trap interface elements 218 may be prepopulated based on thinking traps a typical patient diagnosed with MS may be thinking. In some examples, the particular thinking trap interface elements 218a-218b identified for presentation via the GUI 217 may be based on the feeling selected by the patient 101 via, for example, GUI 204 (see FIG. 2A). The patient 101 may indicate their thinking by selecting one or more corresponding thinking trap interface elements 218a-218b displayed in the thinking traps GUI 217. In the examples shown (e.g., as shown in FIGS. 2F-2H), a first thinking trap interface element 218a (“Overgeneralizing”) indicates that the patient 101 is overgeneralizing, and a second thinking trap interface element 218b (“Catastrophizing”) indicates that the patient 101 is catastrophizing. The thinking trap interface elements 218a-218b do not represent an exhaustive list of all thinking traps interface elements, but rather an exemplary list of thinking trap interface elements that may be included as part of the thinking traps GUI 217. Furthermore, the thinking traps GUI 217 may include other thinking trap interface elements in addition to thinking trap interface elements 218a-218b, or may omit one or more of thinking trap interface elements 218a-218b.

In the example shown, the patient device 102 detects a sixth sequence of inputs, the sixth sequence of inputs including a thinking trap selection input 219a (e.g., touch or spoken) corresponding to a Sounds Like Me Button 245a that corresponds to the thinking trap interface element 218a (“Overgeneralizing”) indicating that the patient 101 is overgeneralizing. In some implementations, the patient 101 can select one or more thinking trap interface elements by selecting more than one Sounds Like Me Buttons 245, each Sounds Like Me Button 245 corresponding to a thinking trap interface element 218. In other implementations, the patient 101 may opt not to select any thinking trap interface elements. In an example in which the patient opts to select one or more thinking trap interface elements, the patient 101 could select the Sounds Like Me Button 245a that corresponds to the thinking trap interface element 218a and a Sounds Like Me Button 245b that corresponds to the thinking trap interface element 218b, indicating that the patient 101 is both overgeneralizing and catastrophizing.

In some implementations, the thinking trap selection input 219a causes the patient application 103 to transmit time-stamped event data 122 to the multiple sclerosis therapy service 160 (FIG. 1) that includes a selection indication indicating that the patient is presently overgeneralizing. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the thinking trap selection input 219 causes the patient application 103 to generate, for display on the patient device 102, a journal interface element of the plurality of journal interface elements 231 (FIG. 2M), the journal interface element indicating the selected thinking trap. In other examples, the thinking trap selection input 219 causes the patient application 103 to modify the already-generated plurality of journal interface elements 231 to indicate the selected thinking trap.

In some examples, a company selection GUI 221 (FIG. 2I) is provided on the display 116 of the patient device 102. The patient application 103 may advance to the company selection GUI 221, according to one example, in response to the patient 101 selecting one or more thinking trap interface elements 218a-218b. In some configurations, the thinking trap selection input 219 selecting the Sounds Like Me button 245 causes the patient application 103 to automatically display the company selection GUI 221. In other configurations, the patient application 103 requires the patient 101 to first confirm the selected thinking trap interface element 218 by selecting a Done button 246 (e.g., as shown in FIG. 2F). In these configurations, the patient application 103 displays the company selection GUI 221 in response to a selection indication indicating selection of the Done button 246.

At FIG. 2I, in some configurations, the patient application 103 causes the patient device 102 to display the company selection GUI 221 that allows a patient 101 to input the company that they were with when they felt the particular feeling. In the examples shown, the company selection GUI 221 provides a plurality of company interface elements 233, each individual company interface element 233a-n being associated with a corresponding person (as identified by relationship type) that the patient 101 may have been with prior to, or when experiencing, the particular feeling. While the example shown depicts interface elements 233a-233e, the patient 101 may view additional company interface elements 233n by scrolling (e.g., via a swipe gesture). The plurality of company interface elements 233 may be prepopulated based on company a typical patient diagnosed with MS may be with when they experience a particular feeling. The patient 101 may indicate the company that they were with when they experienced the particular feeling by selecting the corresponding company interface element 233 displayed in the company selection GUI 221. In the example shown, a first company interface element 233a (“My Self”) indicates that the patient 101 was alone when they experienced the particular feeling, a second company interface element 233b (“My Partner”) indicates that the patient 101 was with their partner when they experienced the particular feeling, a third company interface element 233c (“My Children”) indicates that the patient 101 was with their children when they experienced the particular feeling, a fourth company interface element 233d (“My Sibling”) indicates that the patient 101 was with their sibling when they experienced the particular feeling, and a fifth company interface element 233e (“My Parent”) indicates that the patient 101 was with their parent when they experienced the particular feeling.

The company interface elements 233a-e do not represent an exhaustive list of all company interface elements, but rather an exemplary list of company interface elements that may be included on company selection GUI 221. Furthermore, company selection GUI 221 may include other company interface elements in addition to company interface elements 233a-233e, or may omit one or more of company interface elements 233a-233e.

In the example shown, the patient device 102 detects a seventh sequence of inputs, the seventh sequence of inputs including a company selection input 223 (e.g., touch or spoken) corresponding to the company interface element 223d (“My Sibling”) indicating that the patient 101 was with their sibling when they felt the particular feeling. In some implementations, the company selection input 223 causes the patient application 103 to transmit time-stamped event data 122 to the multiple sclerosis therapy service 160 (FIG. 1) that includes a selection indication indicating that the patient was with their sibling when they felt the particular feeling. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the company selection input 223 causes the patient application 103 to generate, for display on the patient device 102, a journal interface element of the plurality of journal interface elements 231 (FIG. 2M), the journal interface element indicating the selected company. In other examples, the company selection input 223 causes the patient application 103 to modify the already-generated plurality of journal interface elements 231 to indicate the selected company.

In some examples, a location selection GUI 224 (FIG. 2J) is provided on the display 116 of the patient device 102. The patient application 103 may advance to the location selection GUI 224, according to one example, in response to the patient 101 selecting a company interface element 233. In some configurations, the company selection input 223 selecting the company interface element 233 causes the patient application 103 to automatically display the location selection GUI 224. In other configurations, the patient application 103 requires the patient 101 to first confirm the selected company interface element 233 by selecting a Company Selection Done Button 247 (e.g., as shown in FIG. 2I). In these configurations, the patient application 103 displays the location selection GUI 224 in response to a selection indication indicating selection of the Company Selection Done Button 247.

At FIG. 2J, in some configurations, the patient application 103 causes the patient device 102 to display the location selection GUI 224 that allows a patient 101 to input the location that patient 101 was at prior to, or when, the patient 101 felt the particular feeling. In the examples shown, the location selection GUI 224 provides a plurality of location interface elements 225, each individual location interface element 225a-n being associated with a corresponding location that the patient 101 may have been at prior to, or when, experiencing the particular feeling. While the example shown depicts location interface elements 225a-225e, the patient 101 may view additional location interface elements 225n by scrolling (e.g., via a swipe gesture). The plurality of location interface elements 225 may be prepopulated based on locations commonly frequented by patients diagnosed with MS. The patient 101 may indicate the location that they were at prior to, or when, they experienced the particular feeling by selecting the corresponding location interface element 225 displayed in the location selection GUI 224. In the example shown, a first location interface element 225a (“Home”) indicates that the patient 101 was at home when they experienced the particular feeling, a second location interface element 225b (“Doctor”) indicates that the patient 101 was at their doctor's office when they experienced the particular feeling, a third location interface element 225c (“Work”) indicates that the patient 101 was at their work or place of employment when they experienced the particular feeling, a fourth location interface element 225d (“Commute”) indicates that the patient 101 was commuting to and/or from a location when they experienced the particular feeling, and a fifth location interface element 225e (“Store”) indicates that the patient 101 was at a store when they experienced the particular feeling.

The location interface elements 225a-e do not represent an exhaustive list of all location interface elements, but rather an exemplary list of location interface elements that may be included on location selection GUI 224. Furthermore, location selection GUI 224 may include other location interface elements in addition to location interface elements 225a-225e, or may omit one or more of location interface elements 225a-225e.

In the example shown, the patient device 102 detects an eighth sequence of inputs, the eighth sequence of inputs including a location selection input 226 (e.g., touch or spoken) corresponding to the feeling interface element 225d (“Commute”) indicating that the patient 101 was commuting to or from a location when they felt the particular feeling. In some implementations, the location selection input 226 causes the patient application 103 to transmit time-stamped event data 122 to the multiple sclerosis therapy service 160 (FIG. 1) that includes a selection indication indicating that the patient was commuting to or from a location when they experienced the particular feeling. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the location selection input 226 causes the patient application 103 to generate, for display on the patient device 102, a journal interface element of the plurality of journal interface elements 231 (FIG. 2M), the journal interface element indicating the selected location. In other examples, the location selection input 226 causes the patient application 103 to modify the already-generated plurality of journal interface elements 231 to indicate the selected location.

In some examples, a MS symptoms selection GUI 227 (FIG. 2K) is provided on the display 116 of the patient device 102. The patient application 103 may advance to the MS symptoms selection GUI 227, according to one example, in response to the patient 101 selecting a location interface element 225. In some configurations, the location selection input 226 selecting the location interface element 225 causes the patient application 103 to automatically display the MS symptoms selection GUI 227. In other configurations, the patient application 103 requires the patient 101 to first confirm the selected location interface element 225 by selecting a Location Selection Done Button 248. In these configurations, the patient application 103 displays the MS symptom selection GUI 227 in response to a selection indication indicating selection of the Location Selection Done Button 248.

At FIG. 2K, in some configurations, the patient application 103 causes the patient device 102 to display the MS symptom selection GUI 227 that allows a patient 101 to input one or more MS symptoms that they experienced associated with the particular feeling. In the examples shown, the MS symptom selection GUI 227 provides a plurality of MS symptom interface elements 228, each individual MS symptom interface element 228a-n being associated with a corresponding symptom that the patient 101 may have experienced associated with the particular feeling. While the example shown depicts MS symptom interface elements 228a-228h, the patient 101 may view additional MS symptom interface elements 228n by scrolling (e.g., via a swipe gesture). The plurality of MS symptom interface elements 228 may be prepopulated based on MS symptom a patient diagnosed with MS may experience related to the selected feeling (e.g., as selected through GUI 204 shown at FIG. 2A). The patient 101 may indicate the MS symptom that they experienced associated with the particular feeling by selecting the corresponding MS symptom interface element 228 displayed in the MS symptom selection GUI 228. In the example shown, a first MS symptom interface element 228a (“Relapse”) indicates that the patient 101 had a relapse associated with the particular feeling, a second MS symptom interface element 228b (“Fatigue”) indicates that the patient 101 experienced fatigue associated with the particular feeling, a third MS symptom interface element 228c (“Brain Fog”) indicates that the patient 101 experienced brain fog associated with the particular feeling, a fourth MS symptom interface element 228d (“Tremor”) indicates that the patient 101 experienced at least one tremor associated with the particular feeling, a fifth MS symptom interface element 228e (“Focus”) indicates that the patient 101 experienced difficulty focusing associated with the particular feeling, a sixth MS symptom interface element 228f (“Memory”) indicates that the patient 101 experienced memory problems associated with the particular feeling, a seventh MS symptom interface element 228g (“Balance Problems”) indicates that the patient 101 experienced balance problems associated with the particular feeling, and an eighth MS symptom interface element 228h (“Vision”) indicates that the patient 101 experienced vision problems associated with the particular feeling.

The MS symptoms interface elements 228a-h do not represent an exhaustive list of all MS symptom interface elements, but rather an exemplary list of symptom interface elements that may be included on MS symptom selection GUI 227. Furthermore, MS symptom selection GUI 227 may include other symptom interface elements in addition to symptom interface elements 228a-228h, or may omit one or more of MS symptom interface elements 228a-228h.

In the example shown, the patient device 102 detects a ninth sequence of inputs, the ninth sequence of inputs including a MS symptom selection input 229 (e.g., touch or spoken) corresponding to the MS symptom interface element 228d (“Tremor”) indicating that the patient 101 felt one or more tremors when they experienced the particular feeling. In some implementations, the MS symptom selection input 229 causes the patient application 103 to transmit time-stamped event data 122 to the multiple sclerosis therapy service 160 (FIG. 1) that includes a selection indication indicating that the patient felt tremors when they experienced the particular feeling. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the MS symptom selection input 229 causes the patient application 103 to generate, for display on the patient device 102, a journal interface element of the plurality of journal interface elements 231 (FIG. 2M), the journal interface element indicating the selected MS symptom. In other examples, the MS symptom selection input 229 causes the patient application 103 to modify the already-generated plurality of journal interface elements 231 to indicate the selected MS symptom.

In some examples, a journal GUI 230 (FIG. 2M) is provided on the display 116 of the patient device 102. The patient application 103 may advance to the journal GUI 230, according to one example, in response to the patient 101 selecting a MS symptom interface element 228. In some configurations, the MS symptom selection input 229 selecting the MS symptom interface element 228 causes the patient application 103 to automatically display the journal GUI 230. In other configurations, the patient application 103 requires the patient 101 to first confirm the selected MS interface element 228 by selecting a MS Symptoms Selection Done Button 249. In these configurations, the patient application 103 displays the journal GUI 230 in response to a selection indication indicating selection of the MS Symptoms Selection Done Button 249.

At FIG. 2M, in some configurations, the patient application 103 causes the patient device 102 to display the journal GUI 230 that allows a patient 101 to view information corresponding to a history of past interactions between the patient 101 and the patient application 103. In the examples shown, the journal GUI 230 provides a timestamp interface element 232 associated with a particular time and date that the patient application recorded the interaction between the patient 101 and the patient application 103, a plurality of journal interface elements 231, each individual journal interface element being associated with corresponding journal information that the patient 101 may have entered in while interacting with the patient application 103. While the example shown depicts journal interface elements 231a-231h, the patient 101 may view additional journal interface elements 231n by scrolling (e.g., via a swipe gesture). The plurality of journal interface elements 231 may be prepopulated based on interactions between the patient 101 and the patient application 103 at the time and day corresponding to the timestamp interface element 232. The patient 101 may view past interactions between the patient 101 and the patient application 103. In the example shown, at a time and day corresponding to the timestamp interface element 232 (“January 30th 2019, 2:58 pm”), a first journal interface element 231a (“Start Feeling”) indicates that the patient 101 first selected the scared feeling and a feeling intensity of 59%, a second journal interface element 231b (“Who I Was With”) indicates that the patient 101 was alone when they interacted with the patient application 103 at the time and day corresponding to the timestamp interface element 232, a third journal interface element 231c (“Where”) indicates that the patient 101 was at the doctor's office when they interacted with the patient application 103 at the time and day corresponding to the timestamp interface element 232, a fourth journal interface element 231d (“MS Symptoms”) indicates that the patient 101 felt fatigue when they interacted with the patient application 103 at the time and day corresponding to the timestamp interface element 232, a fifth journal interface element 231e (“Automatic Thought”) indicates that the patient 101 had the automatic thought that the patient needs to calm down when they interacted with the patient application 103 at the time and day corresponding to the timestamp interface element 232, a sixth journal interface element 231f (“Thinking Traps”) indicates that the patient 101 overgeneralized when they interacted with the patient application 103 at the time and day corresponding to the timestamp interface element 232, a seventh journal interface element 231g (“Alternative Thought”) indicates that the patient 101 chose the alternative thought that the patient 101 is going to get through this eventually when they interacted with the patient application 103 at the time and day corresponding to the timestamp interface element 232, and an eighth journal interface element 231h (“End Feeling”) indicates the patient feels 41% less scared at the ending of the interaction between the patient 101 and the patient application 103 at the time and day corresponding to the timestamp interface element 232.

The journal interface elements 231a-h do not represent an exhaustive list of all journal interface elements, but rather an exemplary list of journal interface elements that may be included on journal GUI 230. Furthermore, journal GUI 230 may include other journal interface elements in addition to journal interface elements 231a-231h, or may omit one or more of journal interface elements 231a-231h.

At FIG. 2L, in some configurations, the patient application 103 causes the patient device 102 to display a recap interface element 220. This may occur at any point during the interaction between the patient 101 and the patient application 103, but in the example shown, occurs at least after the patient 101 has selected an automatic thought and one or more thinking traps. In the example shown, the recap interface element 220 provides information to patient 101 corresponding to an automatic thought and a thinking trap selected by the patient 101 while the patient 101 interacted with the patient application 103. The information in the recap interface element 220 does not represent an exhaustive list of all information capable of representation in the recap interface element 220, but rather an example of the type of information that can be presented in the recap interface 220. Furthermore, the recap interface 220 may include other information in addition to the information depicted in the example in FIG. 2L, or may omit information depicted in the example in FIG. 2L.

At FIG. 2N, in some configurations, the patient application 103 causes the patient device 102 to display a positive feeling selection GUI 250 that allows the patient 101 to input a particular feeling they are presently experiencing, or has recently experienced. In the example shown, the positive feeling selection GUI 250 provides a plurality of positive feeling interface elements 251, each 251a-n associated with a corresponding feeling the patient 101 is experiencing or has recently experienced. While the example shown depicts interface elements 251a-251h, the patient 101 may view additional interface elements 251n by scrolling (e.g., via a swipe gesture). The plurality of positive feelings interface elements 251 may be prepopulated based on common feelings a typical patient with MS may be experiencing. The patient 101 may indicate their current feelings by selecting the corresponding positive feeling interface element 251 displayed in the positive feeling selection GUI 250. In the example shown, a first positive feeling interface element 251a (“Calm”) indicates that the patient 101 is feeling calm, a second positive feeling interface element 251b (“Okay”) indicates that the patient 101 is feeling okay, a third positive feeling interface element 251c (“Proud”) indicates that the patient 101 is feeling proud, a fourth positive feeling interface element 251d (“Hopeful”) indicates that the patient 101 is feeling hopeful, a fifth positive feeling interface element 251e (“Happy”) indicates that the patient 101 is feeling happy, a sixth positive feeling interface element 251f (“Optimistic”) indicates that the patient 101 is feeling optimistic, a seventh positive feeling interface element 251g (“Determined”) indicates that the patient 101 is feeling determined, and an eighth positive feeling interface element 251h (“Grateful”) indicates that the patient 101 is feeling grateful.

The positive feeling interface elements 251a-251h do not represent an exhaustive list of all positive feeling interface elements, but rather an exemplary list of positive feeling interface elements that may be included as part of the positive feeling selection GUI 250. Furthermore, the positive feeling selection GUI 250 may include other positive feeling interface elements in addition to positive feeling interface elements 251a-251h, or may omit one or more of positive feeling interface elements 251a-251h, without departing from the teachings herein. In some implementations, each of the plurality of positive feeling interface elements 251 is categorized as being associated with one of “Negative” feelings (e.g., FIG. 2A) or “Positive” feelings.

In the example shown, the patient device 102 detects a tenth sequence of inputs, the tenth sequence of inputs including a positive feeling selection input 254 (e.g., touch or spoken) corresponding to the positive feeling element interface 251c (“Proud”) indicating they are feeling proud. In some implementations, the positive feeling selection input 254 causes the patient application 103 to transmit time-stamped event data 122 to the multiple sclerosis therapy service 160 (FIG. 1) that includes a selection indication indicating that the patient is presently feeling proud. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In some examples, the positive feeling selection input 254 causes the patient application 103 to generate, for display on the patient device 102, a positive journal interface element of a plurality of journal interface elements 260 (FIG. 2Q), the positive journal interface element indicating the selected feeling. In other examples, the positive feeling selection input 254 causes the patient application 103 to modify the already-generated plurality of positive journal interface elements 260 to indicate the selected feeling.

After detecting selection of a positive feeling interface element 251, in some embodiments, the patient application 103 advances to display a situation selection GUI 255 (FIG. 2O) on the display 116 of the patient device 102. In some configurations, the positive feeling selection input 254 selecting the positive feeling interface element 251 causes the patient application 103 to automatically display the situation selection GUI 255. In other configurations, the patient application 103 requires the patient 101 to first confirm the selected positive feeling interface element 251 by selecting a Positive Feeling Selection Done Button 253 (e.g., as shown in FIG. 2O). In these configurations, the patient application 103 displays the feeling spectrum GUI 207 in response to a selection indication indicating selection of the Feeling Selection Done Button 237.

At FIG. 2O, in some configurations, the patient application 103 causes the patient device 102 to display the situation selection GUI 255 that allows the patient 101 to input a situation corresponding to what they did. The situation may correspond to an activity the patient 101 did recently. The situation may also correspond to an activity the patient 101 engaged in when the patient 101 felt the selected positive feeling, or when the patient 101 felt a positive feeling. In the example shown, the situation selection GUI 255 provides a plurality of situation interface elements 256, each situation interface element 256a-256n being associated with a corresponding situation that the patient 101 may have recently been involved in, or currently is involved in. While the example shown depicts situation interface elements 256a-256j, the patient 101 may view additional interface elements 256n by scrolling (e.g., via a swipe gesture). The plurality of situation interface elements 256 may be prepopulated based on situations that patients with MS are commonly involved with, or activities that patients with MS commonly partake in. The patient 101 may indicate the situation associated with them by selecting the corresponding situation interface element 256 displayed in the situation selection GUI 255. In the example shown, a first situation interface element 256a (“Catch it, Check it, Change it”) indicates that the patient 101 engaged in the activity of Catch it, Check it, Change it, a second situation interface element 256b (“Meditated”) indicates that the patient 101 meditated, a third situation interface element 256c (“Spent time with a loved one”) indicates that the patient 101 spent time with a loved one, a fourth situation interface element 256d (“Spent time with a pet”) indicates that the patient 101 spent time with a pet, a fifth situation interface element 256e (“Ate healthy”) indicates that the patient 101 ate healthy, a sixth situation interface element 256f (“I got a good check up at the doctor”) indicates that the patient 101 got a good check up at the doctor, a seventh situation interface element 256g (“I accomplished something”) indicates that the patient 101 accomplished something, an eighth situation interface element 256h (“I just feel good”) indicates that the patient 101 just feels good, a ninth situation interface element 256i (“Exercised”) indicates that the patient 101 exercised, a tenth situation interface element 256j (“Yoga”) indicates that the patient 101 did yoga, and an eleventh situation interface element 256k (“Spiritual Activity”) indicates that the patient 101 engaged in a spiritual activity.

The situation interface elements 256a-256k do not represent an exhaustive list of all situation interface elements, but rather an exemplary list of situation interface elements that may be included on the situation selection GUI 255. Furthermore, the situation selection GUI 255 may include other situation interface elements in addition to situation interface elements 256a-256k, or may omit one or more situation interface elements 256a-256k.

In the example shown, the patient device 102 detects an eleventh sequence of inputs, the eleventh sequence of inputs including a situation selection input 257 (e.g., touch or spoken) corresponding to the situation interface 256e (“Ate healthy”) indicating that the patient 101 ate healthy. In some implementations, the situation selection input 257 causes the patient application 103 to transmit time-stamped event data 122 to the multiple sclerosis therapy service 160 (FIG. 1) that includes a selection indication indicating that the patient 101 ate healthy.

In some examples, the situation selection input 257 causes the patient application 103 to generate, for display on the patient device 102, a positive journal interface element of a plurality of journal interface elements 260 (FIG. 2Q), the positive journal interface element indicating the selected situation. In other examples, the situation selection input 257 causes the patient application 103 to modify the already-generated plurality of positive journal interface elements 260 to indicate the selected situation.

At FIG. 2P, in some configurations, the patient application 103 causes the patient device 102 to display a positive reflection element 258. This may occur at any point during the interaction between the patient 101 and the patient application 103, but in the example shown, occurs at least after the patient 101 has selected a positive feeling and a situation. In the example shown, the positive reflection element 258 provides information to patient 101 corresponding to a positive feeling and a situation selected by the patient 101 while the patient 101 interacted with the patient application 103. The information in the positive reflection element 258 does not represent an exhaustive list of all information capable of representation in the positive reflection element 258, but rather an example of the type of information that can be presented in the positive reflection element 258. Furthermore, the positive reflection element 258 may include other information in addition to the information depicted in the example in FIG. 2P, or may omit information depicted in the example in FIG. 2P.

At FIG. 2Q, in some configurations, the patient application 103 causes the patient device 102 to display a positive journal GUI 259 that allows a patient 101 to view information corresponding to a history of past interactions between the patient 101 and the patient application 103. In the examples shown, the positive journal GUI 259 provides a timestamp interface element 261 associated with a particular time and date that the patient application recorded the interaction between the patient 101 and the patient application 103, the plurality of positive journal interface elements 260, each individual positive journal interface element being associated with corresponding journal information that the patient 101 may have entered in while interacting with the patient application 103. While the example shown depicts positive journal interface elements 260a-260e, the patient 101 may view additional positive journal interface elements 260n by scrolling (e.g., via a swipe gesture). The plurality of journal interface elements 260 may be prepopulated based on interactions between the patient 101 and the patient application 103 at the time and day corresponding to the timestamp interface element 261. The patient 101 may view past interactions between the patient 101 and the patient application 103. In the example shown, at a time and day corresponding to the timestamp interface element 261 (“Monday, Jan 7th, 1:00 PM”), a first positive journal interface element 260a (“Feeling”) indicates that the patient 101 felt proud at the time and day corresponding to the timestamp interface element 261, a second positive journal interface element 260b (“Where you were”) indicates that the patient 101 was at their house when they interacted with the patient application 103 at the time and day corresponding to the timestamp interface element 261, a third positive journal interface element 260c (“Who you were with”) indicates that the patient 101 was alone when they interacted with the patient application 103 at the time and day corresponding to the timestamp interface element 261, a fourth positive journal interface element 260d (“Ate Healthy”) indicates that the patient 101 ate healthy when they interacted with the patient application 103 at the time and day corresponding to the timestamp interface element 261, and a fifth positive journal interface element 260e (“Positive Reflection”) indicates that the patient 101 felt proud of themselves for taking control and following through with a healthier diet and believed that it really helped with their symptoms when they interacted with the patient application 103 at the time and day corresponding to the timestamp interface element 261.

The positive journal interface elements 260a-e do not represent an exhaustive list of all journal interface elements, but rather an exemplary list of positive journal interface elements that may be included on the positive journal GUI 259. Furthermore, the positive journal GUI 259 may include other positive journal interface elements in addition to positive journal interface elements 260a-260e, or may omit one or more of positive journal interface elements 260a-260e.

At FIG. 2R, in some configurations, the patient application 103 causes the patient device 102 to display a relax-and-remind GUI 262 that provides a mindfulness interface element 264 and a fatigue interface element 266. This may occur at any point during the interaction between the patient 101 and the patient application 103. In some examples, the patient device 102 displays the relax-and-remind GUI 262 at least after the patient 101 has selected a relax-and-remind selection input.

At FIG. 2S, upon the patient application 103 detecting a mindfulness selection input 265 selecting the mindfulness interface element 264, the patient application 103 is configured to display a mindfulness GUI 268 that provides a plurality of mindfulness technique interface elements 270a-f, each mindfulness technique interface element 270 being associated with a particular mindfulness technique. In some implementations, the mindfulness techniques correspond to current thoughts or emotions experienced by the patient 101. For example, the mindfulness techniques may correspond to stress relief, feeling stressed, resolving shame, going through shame, less lonely now, lingering loneliness, clearing depression, lingering depression, resolving grief, still grieving, surrender frustration, feeling frustrated, goodbye anger, anger persists, less anxious, more anxious, letting go of panic, panic stricken, etc.

The mindfulness technique interface elements 270a-f do not represent an exhaustive list of all mindfulness technique interface elements, but rather an exemplary list that may be included on the mindfulness GUI 268. Furthermore, the mindfulness GUI 268 may include other mindfulness technique interface elements in addition to the mindfulness technique interface elements 270a-f, or may omit one or more of the mindfulness technique interface elements 270a-f.

At FIG. 2T, upon the patient application 103 detecting a mindfulness technique selection input 271 selecting one of the mindfulness technique interface elements 270, e.g., the mindfulness technique interface element 270d corresponding to “Feeling Frustrated,” the patient application 103 is configured to display a mindfulness technique data GUI 272 that provides the data corresponding to the selected mindfulness technique. The plurality of mindfulness techniques may include audio data, video data, audio/video data, interactive data, etc. The mindfulness technique data GUI 272 may provide other interface elements, such as a play/pause button, an “I'm Done” button, etc. While FIG. 2T illustrates a single audio and/or video display, it should be understood that multiple selectable presentations may be presented. In some implementations, the mindfulness technique selection input 271 causes the patient application 103 to transmit time-stamped event data 122 to the multiple sclerosis therapy service 160 (FIG. 1) that includes a selection indication indicating that the patient 101 has or had feelings of being frustrated. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

At FIG. 2U, upon the patient application 103 detecting a fatigue selection input 267 selecting the fatigue interface element 266 (FIG. 2R), the patient application 103 is configured to display a fatigue GUI 274 providing a plurality of fatigue type interface elements 276a-h, each fatigue type interface element 276 being associated with a particular fatigue type that may be experienced by patients suffering from multiple sclerosis. The plurality of fatigue types may correspond to lassitude, diet, sleep, environment, cognitive, emotional, overstimulation, inactivity, heat, etc.

The fatigue type interface elements 276a-h do not represent an exhaustive list of all fatigue type interface elements, but rather an exemplary list that may be included on the fatigue GUI 274. Furthermore, the fatigue GUI 274 may include other fatigue type interface elements in addition to the fatigue type interface elements 276a-h, or may omit one or more of the fatigue type interface elements 276a-h.

At FIG. 2V, upon the patient application 103 detecting a fatigue type selection input 277 selecting one of the fatigue type interface elements 276, e.g., the fatigue type interface element 276c corresponding to “Sleep,” the patient application 103 is configured to display a fatigue type data GUI 278 that provides the data corresponding to the selected fatigue type. In some examples, the data corresponding to the selected fatigue type includes a plurality of presentations 280a-c. The plurality of fatigue types may include audio data, video data, audio/video data, interactive data, etc. The fatigue type data GUI 278 may provide other interface elements, such as a play/pause button, an “I'm Done” button, etc. In some implementations, the fatigue type selection input 277 causes the patient application 103 to transmit time-stamped event data 122 to the multiple sclerosis therapy service 160 (FIG. 1) that includes a selection indication indicating that the patient 101 has or had feelings of being frustrated. As noted previously, by sending the time-stamped event data 122 to the multiple sclerosis therapy service 160, then a log of the patient's inputs into the interface can be maintained, for example for diagnostic or research purposes, or to allow tracking of the progress of the digital therapy.

In implementations where the mindfulness techniques provide audio and/or video data and the fatigue types provide audio and/or video data, the audio and/or video data may be presented by a patient suffering from multiple sclerosis to provide a sense of community and empathy that may not be exhibited through use of, e.g., a paid actor.

FIG. 3 is a flow chart illustrating a method 300 for treating depressive symptoms associated with multiple sclerosis in accordance with an example implementation of the disclosed technology. According to one example, the method 300 may be performed by an electronic device, such as the patient device 102. The method 300 begins at block 302 where a feeling selection interface (e.g., the feeling selection GUI 204) is displayed. The feeling selection interface presents a plurality of feeling interface elements (e.g., the plurality of feeling interface elements 205), each feeling interface element being associated with a particular feeling. At block 304, a first sequence of inputs including a feeling selection input (e.g., the feeling selection input 206) is received. The feeling selection input corresponds to a particular feeling interface element (e.g., the second feeling interface element 205b). At block 306, the electronic device displays a feeling spectrum interface (e.g., the feeling spectrum GUI 207). The feeling spectrum interface presents a plurality of intensities (e.g., the plurality of intensities 208) associated with the particular feeling.

At block 308, the electronic device receives a second sequence of inputs including a first feeling intensity input (e.g., the first feeling intensity input 209). The first feeling intensity input corresponds to a first intensity (e.g., the third intensity 208c) of the plurality of intensities. At block 310, the electronic device displays an automatic thought selection interface (e.g., the automatic thought selection GUI 210). The automatic thought selection interface presenting a plurality of automatic thought interface elements (e.g., the plurality of automatic thought interface elements 211). Each automatic thought interface element is associated with a particular automatic thought. At block 312, the electronic device receives a third sequence of inputs including an automatic thought selection input (e.g., the automatic thought selection input 212). The automatic thought selection input corresponds to a particular automatic thought interface element. At block 314, the electronic device displays an alternative thought selection interface (e.g., the alternative thought selection GUI 213). The alternative thought selection interface presents a plurality of alternative thought interface elements (e.g., the plurality of alternative thought interface elements 214). Each alternative thought interface element is associated with a particular alternative thought.

At block 316, the electronic device receives a fourth sequence of inputs including an alternative thought selection input (e.g., the alternative thought selection input 215). The alternative thought selection input corresponds to a particular alternative thought interface element. At block 318, the electronic device displays the feeling spectrum interface. At block 320, the electronic device receives a fifth sequence of inputs including a second feeling intensity input (e.g., the second feeling intensity input 216). The second feeling intensity input corresponds to a second intensity (e.g., the fifth intensity 208e) of the plurality of intensities. At block 322, the electronic device generates a journal entry (e.g., the eighth journal interface element 231h). The journal entry indicates at least any difference between the first feeling intensity input and the second feeling intensity input. Following block 322, the method 300 concludes.

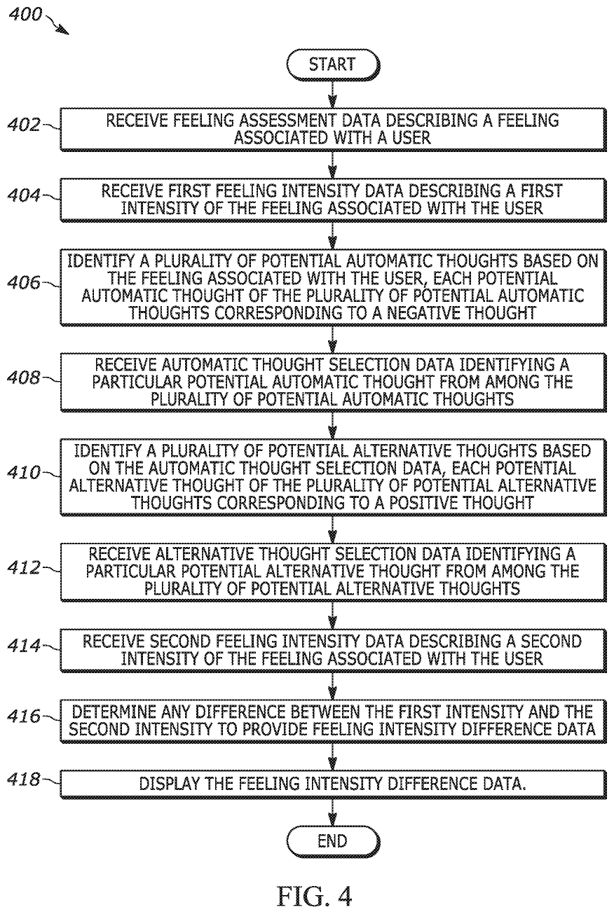

FIG. 4 is a flow chart illustrating another method 400 for treating depressive symptoms associated with multiple sclerosis in accordance with an example implementation of the disclosed technology. According to one example, the method 400 may be performed by an electronic device, such as the patient device 102. The method 400 begins at block 402 where the electronic device receives feeling assessment data describing a feeling associated with a user (e.g., as shown in FIG. 2A). At block 404, the electronic device receives first feeling intensity data describing a first intensity of the feeling associated with the user (e.g., as shown in FIG. 2B).

At block 406, the electronic device identifies a plurality of potential automatic thoughts based on the feeling associated with the user (e.g., as shown in FIG. 2C). Each potential automatic thought of the plurality of potential automatic thoughts correspond to a negative thought. At block 408, the electronic device receives automatic thought selection data identifying a particular potential automatic thought from among the plurality of potential automatic thoughts (e.g., as shown in FIG. 2C).

At block 410, the electronic device identifies a plurality of potential alternative thoughts based on the automatic thought selection data (e.g., as shown in FIG. 2D). Each potential alternative thought of the plurality of potential alternative thoughts correspond to a positive thought. At block 412, the electronic device receives alternative thought selection data identifying a particular potential alternative thought from among the plurality of potential alternative thoughts (e.g., as shown in FIG. 2D).

At block 414, the electronic device receives second feeling intensity data describing a second intensity of the feeling associated with the user (e.g., as shown in FIG. 2E). At block 416, the electronic device determines any difference between the first intensity and the second intensity to provide feeling intensity difference data. At block 418, the electronic device displays the feeling intensity difference data (e.g., as shown in FIG. 2M). Following block 418, the method 400 concludes.

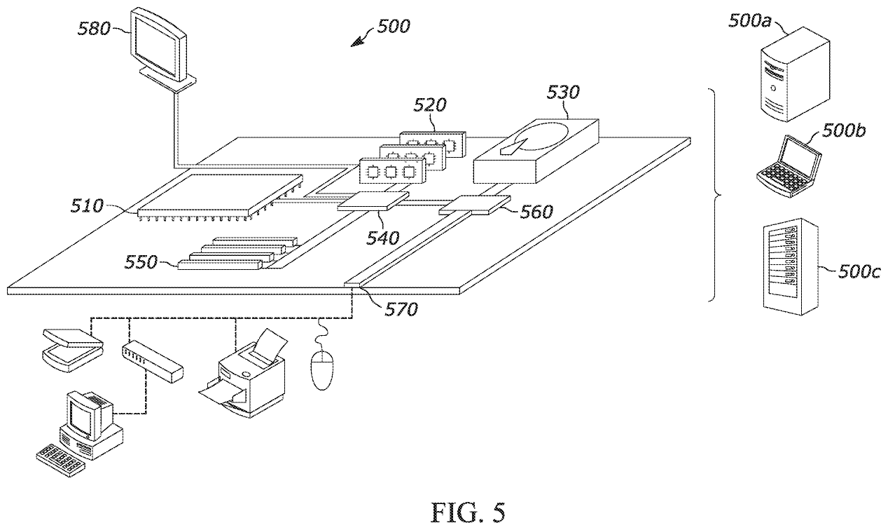

FIG. 5 is schematic view of an example electronic device 500 (e.g., a computing device) that may be used to implement the systems and methods described in this document. The electronic device 500 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The electronic device 500 includes a processor 510, memory 520, a storage device 530, a high-speed interface/controller 540 connecting to the memory 520 and high-speed expansion ports 550, and a low speed interface/controller 560 connecting to a low speed bus 570 and a storage device 530. Each of the components 510, 520, 530, 540, 550, and 560, is interconnected using various busses, and may be mounted on a common motherboard or in other manners as appropriate. The processor 510 can process instructions for execution within the electronic device 500, including instructions stored in the memory 520 or on the storage device 530 to display graphical information for a graphical user interface (GUI) on an external input/output device, such as display 580 coupled to high speed interface 540. In other implementations, multiple processors and/or multiple buses may be used, as appropriate, along with multiple memories and types of memory. Also, multiple electronic device 500 may be connected, with each device providing portions of the necessary operations (e.g., as a server bank, a group of blade servers, or a multi-processor system).

The memory 520 stores information non-transitorily within the electronic device 500. The memory 520 may be a computer-readable medium, a volatile memory unit(s), or non-volatile memory unit(s). The non-transitory memory 520 may be physical devices used to store programs (e.g., sequences of instructions) or data (e.g., program state information) on a temporary or permanent basis for use by the electronic device 500. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 530 is capable of providing mass storage for the electronic device 500. In some implementations, the storage device 530 is a computer-readable medium. In various different implementations, the storage device 530 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 520, the storage device 530, or memory on processor 510.

The high speed controller 540 manages bandwidth-intensive operations for the electronic device 500, while the low speed controller 560 manages lower bandwidth-intensive operations. Such allocation of duties is exemplary only. In some implementations, the high-speed controller 540 is coupled to the memory 520, the display 580 (e.g., through a graphics processor or accelerator), and to the high-speed expansion ports 550, which may accept various expansion cards (not shown).

The electronic device 500 may be implemented in a number of different forms, as shown in the figure. For example, it may be implemented as a standard server 500a or multiple times in a group of such servers 500a, as a laptop computer 500b, or as part of a rack server system 500c.

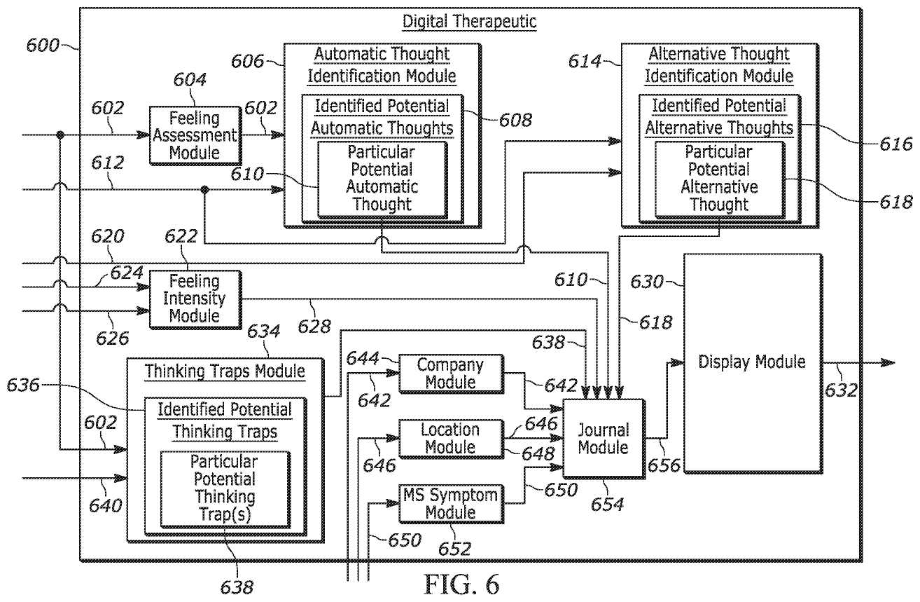

Referring now to FIG. 6, one example of a digital therapeutic 600 for treating depressive symptoms associated with multiple sclerosis is illustrated, in functional block form. As shown, the digital therapeutic 600 includes a feeling assessment module 604, an automatic thought identification module 606, an alternative thought identification module 614, a feeling intensity module 622, a thinking traps module 634, a company module 644, a location module 648, a multiple sclerosis symptom module 652, a journal module 654, and a display module 630. According to one example, the digital therapeutic 600 may be implemented as a computer program executed on an electronic device, such as device 102. According to this example, executing the computer program on the electronic device may serve to administer therapeutic treatment to a user of the electronic device in a manner designed to mitigate, or alleviate, depressive symptoms associated with multiple sclerosis.

In operation, the digital therapeutic 600 may function as follows. The feeling assessment module 604 is configured to receive feeling assessment data 602 (e.g. input 206; block 304). The feeling assessment data 602 may constitute data describing a feeling associated with a user (e.g., anxious, scared, dreadful, etc.). According to one example, the feeling assessment data 602 may be provided to the feeling assessment module 604 via user input as discussed, for example, with regard to FIG. 2A above.

The automatic thought identification module 606 is configured to receive the feeling assessment data 602 from the feeling assessment module 604. In addition, the automatic thought identification module 606 is configured to identify a plurality of potential automatic thoughts 608 based on the feeling assessment data 602. By way of example and not limitation, the plurality of potential automatic thoughts 608 may be identified from within a database or the like (not shown) storing a variety of automatic thoughts. Each potential automatic thought of the plurality of potential automatic thoughts 608 may correspond to a negative thought (although, according to some examples, one or more potential automatic thoughts may correspond to a positive thought). Further, the automatic thought identification module 606 is configured to receive automatic thought selection data 612 (e.g., input 212; block 312). The automatic thought selection data 612 may identify a particular potential automatic thought 610 from among the plurality of potential automatic thoughts 608. According to one example, the automatic thought selection data 612 may be provided to the automatic thought identification module 606 via user input as discussed, for example, with regard to FIG. 2C above.

The alternative thought identification module 614 is configured to receive the automatic thought selection data 612. In addition, the alternative thought identification module 614 is configured to identify a plurality of potential alternative thoughts 616 based on the automatic thought selection data 612. By way of example and not limitation, the plurality of potential alternative thoughts 616 may be identified from within a database or the like (not shown) storing a variety of alternative thoughts. Each potential alternative thought of the plurality of potential alternative thoughts 616 may correspond to a positive thought. Further, the alternative thought identification module 614 is configured to receive alternative thought selection data 620 (e.g., input 215; block 316). The alternative thought selection data 620 may identify a particular potential alternative thought 618 from among the plurality of potential alternative thoughts 616. According to one example, the alternative thought selection data 620 may be provided to the alternative thought identification module 614 via user input as discussed, for example, with regard to FIG. 2D above.

The feeling intensity module 622 is configured to receive first feeling intensity data 624 and second feeling intensity data 626 (e.g., input 209 and input 216; block 308 and block 320). The first feeling intensity data 624 may describe a first intensity of the feeling associated with the user (e.g., as indicated by the feeling assessment data 602) being treated via the digital therapeutic 600 at a first point in time. The second feeling intensity data 626 may describe a second intensity of the feeling associated with the user at a second point in time. According to one example, the second point in time is later than the first point in time. According to one example, the first feeling intensity data 624 may be provided to the feeling intensity module 622 via user input as discussed, for example, with regard to FIG. 2B above. Similarly, in one example, the second feeling intensity data 626 may be provided to the feeling intensity module 622 via user input as discussed, for example, with regard to FIG. 2E above.

In response to receiving the first feeling intensity data 624 and the second feeling intensity data 626, the feeling intensity module is configured to generate feeling intensity difference data 628 (e.g., interface element 231h of FIG. 2M; block 322). The feeling intensity difference data 628 may indicate any difference (including, in some examples, no difference) between the first feeling intensity data 624 and the second feeling intensity data 626. For example, and as discussed with respect to element 231h of FIG. 2M above, the feeling intensity difference data 628 may indicate a change (e.g., a drop) in the intensity of a particular feeling experienced by the user receiving treatment via the digital therapeutic 600.

The thinking traps module 634 is configured to receive the automatic thought selection data 612. In addition, the thinking traps module 634 is configured to identify a plurality of potential thinking traps 636 based on the feeling assessment data 602. By way of example and not limitation, the plurality of potential thinking traps 636 may be identified from within a database or the like (not shown) storing a variety of thinking traps. Each potential thinking trap of the plurality of potential thinking traps 636 may correspond to a negative emotional tendency, such as overgeneralizing, catastrophizing, etc. Further, the thinking traps module 634 is configured to receive thinking trap selection data 640 (e.g., input 219). The thinking trap selection data 640 may identify a particular potential thinking trap 638 from among the plurality of potential thinking traps 636. According to one example, the thinking trap selection data 640 may be provided to the thinking traps module 634 via user input as discussed, for example, with regard to FIGS. 2F-2H above.

The company module 644 is configured to receive company selection data 642 (e.g., input 223). The company selection data 642 may identify, by relationship type (e.g., partner, children, sibling, parent, friend, co-worker, etc.), a person who accompanied a user of the digital therapeutic 600 at a time in which the user experienced the feeling described by the feeling assessment data 602, or, whether the user was alone when they experienced the feeling described by the feeling assessment data 602. As discussed in additional detail below, in some examples, the company selection data 642 may be provided to the journal module 654 for use in generating a journal entry 656.

The location module 648 is configured to receive location selection data 646 (e.g., input 226). The location selection data 646 may identify a location (e.g., home, doctor, work, commute, store, etc.) of the user at the time in which the user experienced the feeling described by the feeling assessment data 602. As discussed in additional detail below, in some examples, the location selection data 646 may be provided to the journal module 654 for use in generating a journal entry 656.

The multiple sclerosis symptom module 652 is configured to receive multiple sclerosis symptom selection data 650 (e.g., input 229). The multiple sclerosis symptom selection data 650 may identify one or more multiple sclerosis symptoms (e.g., relapse, fatigue, brain fog, tremor, focus, memory, balance problems, vision problems, etc.) associated with the user. As discussed in additional detail below, in some examples, the multiple sclerosis symptom selection data 650 may be provided to the journal module 654 for use in generating a journal entry 656.

The journal module 654 is configured to receive the company selection data 642, location selection data 646, multiple sclerosis symptom selection data 650, the particular potential thinking trap 638, the feeling intensity difference data 628, particular potential automatic thought 610, and the particular potential alternative thought 618. In response to receiving one or more of the foregoing types of data, the journal module 654 is configured to generate a journal entry 656 including some or all of the foregoing types of data. On example of a generated journal entry 656 is shown with regard to FIG. 2M and discussed above.

The display module 630 is configured to receive the generated journal entry 656 and generate display data 632 representing the generated journal entry 656. For example, according to one embodiment, the display module 630 is configured to generate display data 632 representing a generated journal entry 656 that includes all of the following types of data: company selection data 642, location selection data 646, multiple sclerosis symptom selection data 650, particular potential thinking trap 638, feeling intensity difference data 628, particular potential automatic thought 610, and particular potential alternative thought 618, as shown, for example, in FIG. 2M. According to another embodiment, the display module 630 is configured to generate display data 632 representing a generated journal entry 656 that includes some, but not all, of the foregoing types of data. Regardless, the generated display data 632 may take the form of pixel data or the like capable of generating an image on a suitable display device, such as display 116 discussed above with regard to FIG. 1.

Among other advantages, the present disclosure provides electronic devices and methods for implementing a prescription digital therapeutic configured to treat depressive symptoms associated with MS. The digital therapeutic may administer cognitive behavioral therapy (CBT) to treat the depressive symptoms. More specifically, the digital therapeutic may implement both cognitive therapy as well as behavioral activation as part of the administered CBT. Administration of CBT via the digital therapeutics described herein may serve to correct distorted cognitions that can cause patients to have a negative view of themselves, the world, and the future.

The present disclosure also provides a digital therapeutic that includes a plurality of GUIs to help a user/patient understand situations, symptoms, and automatic thoughts related to their negative feelings; check their thoughts against a set of common cognitive distortions or “thinking traps”; and identify alternative thoughts that are more helpful and realistic. The patient/user may be provided with examples of automatic and alternative thoughts that were obtained from a large sample of people with MS.

The present disclosure also provides a digital therapeutic to help patients/users focus on developing skills to cope with MS symptoms, such as brain fog and fatigue, related to depression. The digital therapeutic of the present disclosure provides 24/7 access to support and resources for treating depressive symptoms associated with MS.

The present disclosure also provides a digital therapeutic to reduce depressive symptoms associated with multiple sclerosis according to clinical measurements. For example, the digital therapeutic described herein improves patient condition according to one or more of the following clinical measurements: MADRS, BDI-II, and PHQ-9. For example, the digital therapeutic described herein creates physiological changes in patients.

Certain implementations of the disclosed technology are described above with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some implementations of the disclosed technology.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

Although the following description uses terms “first,” “second,” etc. to describe various elements, these elements should not be limited by the terms. These terms are only used to distinguish one element from another. For example, a first touch could be termed a second touch, and, similarly, a second touch could be termed a first touch, without departing from the scope of the various described embodiments. The first touch and the second touch are both touches, but they are not the same touch.

Various implementations of the electronic devices, systems, techniques, and modules described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage resource, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any to signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations of Example 1 have been described. Nevertheless, it will be understood that various modifications may be made without departing from the spirit and scope of the disclosure. Accordingly, other implementations are within the scope of the above embodiments and following claims, including the following implementations, expressed as interrelated items:

Item A1. An electronic device for treating depressive symptoms associated with multiple sclerosis, the electronic device comprising:
a display;
an input device;
one or more processors; and
memory storing one or more programs configured to be executed by the one or more processors, the one or more programs including instructions for:
   displaying, on the display, a feeling selection interface, the feeling selection interface presenting a plurality of feeling interface elements, each feeling interface element being associated with a particular feeling;
   while displaying the feeling selection interface, receiving, via the input device, a first sequence of inputs, the first sequence of inputs including a feeling selection input, the feeling selection input corresponding to a particular feeling interface element;
   in response to receiving the feeling selection input, displaying, on the display, a feeling spectrum interface, the feeling spectrum interface presenting a plurality of intensities associated with the particular feeling;
   while displaying the feeling spectrum interface, receiving, via the input device, a second sequence of inputs, the second sequence of inputs including a first feeling intensity input, the first feeling intensity input corresponding to a first intensity of the plurality of intensities;
   in response to receiving the first feeling intensity input, displaying, on the display, an automatic thought selection interface, the automatic thought selection interface presenting a plurality of automatic thought interface elements, each automatic thought interface element being associated with a particular automatic thought;
   while displaying the automatic thought selection interface, receiving, via the input device, a third sequence of inputs, the third sequence of inputs including an automatic thought selection input, the automatic thought selection input corresponding to a particular automatic thought interface element;
   in response to receiving the automatic thought selection input, displaying, on the display, an alternative thought selection interface, the alternative thought selection interface presenting a plurality of alternative thought interface elements, each alternative thought interface element being associated with a particular alternative thought;
   while displaying the alternative thought selection interface, receiving, via the input device, a fourth sequence of inputs, the fourth sequence of inputs including an alternative thought selection input, the alternative thought selection input corresponding to a particular alternative thought interface element;
   in response to receiving the alternative thought selection input, displaying, on the display, the feeling spectrum interface;
   while displaying the feeling spectrum interface, receiving, via the input device, a fifth sequence of inputs, the fifth sequence of inputs including a second feeling intensity input, the second feeling intensity input corresponding to a second intensity of the plurality of intensities; and
   generating, for display on the display, a journal entry, the journal entry indicating at least any difference between the first feeling intensity input and the second feeling intensity input.
      Item A2. The electronic device of Item A1, wherein the one or more programs further include instructions for:
in response to receiving the automatic thought selection input:
   displaying, on the display, a thinking traps interface, the thinking traps interface presenting a plurality of thinking trap interface elements associated with the particular automatic thought interface element, each thinking trap interface element being associated with a particular thinking trap;
   while displaying the thinking traps interface, receiving, via the input device, a sixth sequence of inputs, the sixth sequence of inputs including one or more thinking trap selection inputs, the one or more thinking trap selection inputs corresponding to one or more particular thinking trap interface elements; and
   wherein the journal entry is modified to further indicate the one or more particular thinking trap interface elements.
      Item A3. The electronic device of Item A2, wherein the one or more programs further include instructions for:
in response to receiving the one or more thinking trap selection inputs, displaying, on the display, a quick recap interface element, the quick recap interface element indicating the particular automatic thought and the one or more particular thinking trap elements.
   Item A4. The electronic device as in any of Items A1-A3, wherein the journal entry is modified to further indicate the particular alternative thought interface element.
   Item A5. The electronic device as in any of Items A1-A4, wherein the one or more programs further include instructions for:
in response to receiving the feeling selection input:
   displaying, on the display, a company selection interface, the company selection interface presenting a plurality of company interface elements, each company interface element being associated with a particular relationship type;
   while displaying the company selection interface, receiving, via the input device, a seventh sequence of inputs, the seventh sequence of inputs including a company selection input, the company selection input corresponding to a particular company interface element; and
   wherein the journal entry is modified to further indicate the particular company interface element.
      Item A6. The electronic device as in any of Items A1-A5, wherein the one or more programs further include instructions for:
in response to receiving the feeling selection input:
   displaying, on the display, a location selection interface, the location selection interface presenting a plurality of location interface elements, each location interface element being associated with a particular location;
   while displaying the location selection interface, receiving, via the input device, an eighth sequence of inputs, the eighth sequence of inputs including a location selection input, the location selection input corresponding to a particular location interface element; and
   wherein the journal entry is modified to further indicate the particular location interface element.
      Item A7. The electronic device as in any of Items A1-A6, wherein the one or more programs further include instructions for:
in response to receiving the feeling selection input:
   displaying, on the display, a multiple sclerosis symptoms selection interface, the multiple sclerosis symptoms selection interface presenting a plurality of multiple sclerosis symptom interface elements, each multiple sclerosis symptom interface element being associated with a particular multiple sclerosis symptom;
   while displaying the multiple sclerosis symptoms selection interface, receiving, via the input device, a ninth sequence of inputs, the ninth sequence of inputs including one or more multiple sclerosis symptom selection inputs, the one or more multiple sclerosis symptom selection inputs corresponding to one or more particular multiple sclerosis symptom interface elements; and
   wherein the journal entry is modified to further indicate the one or more particular multiple sclerosis symptom interface elements.
      Item A8. A computerized method for treating depressive symptoms associated with multiple sclerosis, the method comprising:
at an electronic device including a display and an input device:
   displaying, on the display, a feeling selection interface, the feeling selection interface presenting a plurality of feeling interface elements, each feeling interface element being associated with a particular feeling;
   while displaying the feeling selection interface, receiving, via the input device, a first sequence of inputs, the first sequence of inputs including a feeling selection input, the feeling selection input corresponding to a particular feeling interface element;
   in response to receiving the feeling selection input, displaying, on the display, a feeling spectrum interface, the feeling spectrum interface presenting a plurality of intensities associated with the particular feeling;
   while displaying the feeling spectrum interface, receiving, via the input device, a second sequence of inputs, the second sequence of inputs including a first feeling intensity input, the first feeling intensity input corresponding to a first intensity of the plurality of intensities;
   in response to receiving the first feeling intensity input, displaying, on the display, an automatic thought selection interface, the automatic thought selection interface presenting a plurality of automatic thought interface elements, each automatic thought interface element being associated with a particular automatic thought;
   while displaying the automatic thought selection interface, receiving, via the input device, a third sequence of inputs, the third sequence of inputs including an automatic thought selection input, the automatic thought selection input corresponding to a particular automatic thought interface element;
   in response to receiving the automatic thought selection input, displaying, on the display, an alternative thought selection interface, the alternative thought selection interface presenting a plurality of alternative thought interface elements, each alternative thought interface element being associated with a particular alternative thought;
   while displaying the alternative thought selection interface, receiving, via the input device, a fourth sequence of inputs, the fourth sequence of inputs including an alternative thought selection input, the alternative thought selection input corresponding to a particular alternative thought interface element;
   in response to receiving the alternative thought selection input, displaying, on the display, the feeling spectrum interface;
   while displaying the feeling spectrum interface, receiving, via the input device, a fifth sequence of inputs, the fifth sequence of inputs including a second feeling intensity input, the second feeling intensity input corresponding to a second intensity of the plurality of intensities; and
   generating, for display on the display, a journal entry, the journal entry indicating at least any difference between the first feeling intensity input and the second feeling intensity input.
      Item A9. The computerized method of Item A8, further comprising:
in response to receiving the automatic thought selection input:
   displaying, on the display, a thinking traps interface, the thinking traps interface presenting a plurality of thinking trap interface elements associated with the particular automatic thought interface element, each thinking trap interface element being associated with a particular thinking trap;
   while displaying the thinking traps interface, receiving, via the input device, a sixth sequence of inputs, the sixth sequence of inputs including one or more thinking trap selection inputs, the one or more thinking trap selection inputs corresponding to one or more particular thinking trap interface elements; and
   wherein the journal entry is modified to further indicate the one or more particular thinking trap interface elements.
      Item A10. The computerized method of Item A9, further comprising:
in response to receiving the one or more thinking trap selection inputs, displaying, on the display, a quick recap interface element, the quick recap interface element indicating the particular automatic thought and the one or more particular thinking trap elements.
   Item A11. The computerized method as in any one of Items A8-A10, wherein the journal entry is modified to further indicate the particular alternative thought interface element.
   Item A12. The computerized method as in any one of Items A8-A11, further comprising:
in response to receiving the feeling selection input:
   displaying, on the display, a company selection interface, the company selection interface presenting a plurality of company interface elements, each company interface element being associated with a particular relationship type;
   while displaying the company selection interface, receiving, via the input device, a seventh sequence of inputs, the seventh sequence of inputs including a company selection input, the company selection input corresponding to a particular company interface element and
   wherein the journal entry is modified to further indicate the particular company interface element.
      Item A13. The computerized method as in any one of Items A8-A12, further comprising:
in response to receiving the feeling selection input:
   displaying, on the display, a location selection interface, the location selection interface presenting a plurality of location interface elements, each location interface element being associated with a particular location;
   while displaying the location selection interface, receiving, via the input device, an eighth sequence of inputs, the eighth sequence of inputs including a location selection input, the location selection input corresponding to a particular location interface element; and
   wherein the journal entry is modified to further indicate the particular location interface element.
      Item A14. The computerized method as in any one of claims Items A8-A13, further comprising:
in response to receiving the feeling selection input:
   displaying, on the display, a multiple sclerosis symptoms selection interface, the multiple sclerosis symptoms selection interface presenting a plurality of multiple sclerosis symptom interface elements, each multiple sclerosis symptom interface element being associated with a particular multiple sclerosis symptom;
   while displaying the multiple sclerosis symptoms selection interface, receiving, via the input device, a ninth sequence of inputs, the ninth sequence of inputs including one or more multiple sclerosis symptom selection inputs, the one or more multiple sclerosis symptom selection inputs corresponding to one or more particular multiple sclerosis symptom interface elements; and
   wherein the journal entry is modified to further indicate the one or more particular multiple sclerosis symptom interface elements.
      Item A15. The computerized method as in any one of claims Items A8-A14, further comprising:
in response to receiving a mindfulness selection input, displaying, on the display, a mindfulness technique interface, the mindfulness technique interface presenting a plurality of mindfulness technique interface elements, each mindfulness technique interface element being associated with a particular mindfulness technique; and
in response to receiving a mindfulness technique selection input indicating selection of a mindfulness technique interface element corresponding to a particular mindfulness technique, displaying, on the display, mindfulness data corresponding to the particular mindfulness technique, the mindfulness data including at least one of audio, video, or interactive data.
   Item A16. The computerized method as in any one of claims Items A8-A15, further comprising:
in response to receiving a fatigue selection input, displaying, on the display, a fatigue type interface, the fatigue type interface presenting a plurality of fatigue type interface elements, each fatigue type interface element being associated with a particular fatigue type; and
in response to receiving a fatigue type selection input indicating selection of a fatigue type interface element corresponding to a particular fatigue type, displaying, on the display, fatigue type data corresponding to the particular fatigue type, the fatigue type data including at least one of audio, video, or interactive data.
   Item A17. A computerized method for treating depressive symptoms associated with multiple sclerosis, the method comprising:
at an electronic device including a display and an input device:
   receiving, via the input device, feeling assessment data, the feeling assessment data describing a feeling associated with a user;
   receiving, via the input device, first feeling intensity data, the first feeling intensity data describing a first intensity of the feeling associated with the user;
   identifying a plurality of potential automatic thoughts based on the feeling associated with the user, each potential automatic thought of the plurality of potential automatic thoughts corresponding to a negative thought;
   receiving, via the input device, automatic thought selection data, the automatic thought selection data identifying a particular potential automatic thought from among the plurality of potential automatic thoughts;
   identifying a plurality of potential alternative thoughts based on the automatic thought selection data, each potential alternative thought of the plurality of potential alternative thoughts corresponding to a positive thought;
   receiving, via the input device, alternative thought selection data, the alternative thought selection data identifying a particular potential alternative thought from among the plurality of potential alternative thoughts;
   receiving, via the input device, second feeling intensity data, the second feeling intensity data describing a second intensity of the feeling associated with the user; and
   determining any difference between the first intensity and the second intensity to provide feeling intensity difference data; and
   displaying, on the display, the feeling intensity difference data.
      Item A18. An electronic device, comprising:
a display;
an input device;
one or more processors; and
   memory storing one or more programs configured to be executed by the one or more processors, the one or more programs including instructions for:
receiving, via the input device, feeling assessment data, the feeling assessment data describing a feeling associated with a user;
receiving, via the input device, first feeling intensity data, the first feeling intensity data describing a first intensity of the feeling associated with the user;
identifying a plurality of potential automatic thoughts based on the feeling associated with the user, each potential automatic thought of the plurality of potential automatic thoughts corresponding to a negative thought;
receiving, via the input device, automatic thought selection data, the automatic thought selection data identifying a particular potential automatic thought from among the plurality of potential automatic thoughts;
identifying a plurality of potential alternative thoughts based on the automatic thought selection data, each potential alternative thought of the plurality of potential alternative thoughts corresponding to a positive thought;
receiving, via the input device, alternative thought selection data, the alternative thought selection data identifying a particular potential alternative thought from among the plurality of potential alternative thoughts;
receiving, via the input device, second feeling intensity data, the second feeling intensity data describing a second intensity of the feeling associated with the user; and
determining any difference between the first intensity and the second intensity to provide feeling intensity difference data; and
displaying, on the display, the feeling intensity difference data.
   Item A19. A digital therapeutic for treating depressive symptoms associated with multiple sclerosis, the digital therapeutic comprising:
an automatic thought identification module, the automatic thought identification module configured to (i) identify a plurality of potential automatic thoughts based on feeling assessment data describing a feeling associated with a user, each potential automatic thought of the plurality of potential automatic thoughts corresponding to a negative thought and (ii) receive automatic thought selection data identifying a particular potential automatic thought from among the plurality of potential automatic thoughts;
an alternative thought identification module, the alternative thought identification module configured to (i) identify a plurality of potential alternative thoughts based on the automatic thought selection data, each potential alternative thought of the plurality of potential alternative thoughts corresponding to a positive thought and (ii) receive alternative thought selection data identifying a particular potential alternative thought from among the plurality of potential alternative thoughts;
a feeling intensity module, the feeling intensity module configured to (i) receive first feeling intensity data describing a first intensity of the feeling associated with the user at a first point in time; (ii) receive second feeling intensity data describing a second intensity of the feeling associated with the user at a second point in time, the second point in time being later than the first point in time; and (iii) generate feeling intensity difference data, the feeling intensity difference data indicating any difference between the first intensity and the second intensity; and
a display module, the display module configured to generate display data representing the feeling intensity difference data.
   Item A20. The digital therapeutic of Item A19, further comprising:
a feeling assessment module, the feeling assessment module configured to receive the feeling assessment data describing the feeling associated with the user.
   Item A21. The digital therapeutic of Item A19, further comprising:
a thinking traps module, the thinking traps module configured to (i) identify a plurality of potential thinking traps based on the feeling assessment data and (ii) receive thinking trap selection data identifying one or more particular potential thinking traps from among the plurality of potential thinking traps.
   Item A22. The digital therapeutic as in any one of Items A19-A21, further comprising:
a journal module, the journal module configured to generate a journal entry comprising at least the feeling intensity difference data.
   Item A23. The digital therapeutic of Item A22, further comprising:
a company module, the company module configured to receive company selection data identifying, by relationship type, a person who accompanied the user at a time in which the user experienced the feeling; and
wherein the journal entry further comprises the company selection data.
   Item A24. The digital therapeutic as in any one of Items A22-A23, further comprising:
a location module, the location module configured to receive location selection data identifying a location of the user at a time in which the user experienced the feeling; and
wherein the journal entry further comprises the location selection data.
   Item A25. The digital therapeutic as in any one of Items A22-A24, further comprising:
a multiple sclerosis symptom module, the multiple sclerosis symptom module configured to receive multiple sclerosis symptom selection data identifying one or more multiple sclerosis symptoms associated with the user; and
wherein the journal entry further comprises the multiple sclerosis symptom selection data.
   Item A26. The digital therapeutic as in any one of Items A22-A25, wherein the journal entry further comprises the thinking trap selection data.
   Item A27. A non-transitory computer-readable storage medium storing one or more programs configured to be executed by one or more processors of an electronic device with a display and input device, the one or more programs including instructions for performing the method of any of Items A8-A16.
   Item A28. A non-transitory computer-readable storage medium storing one or more programs configured to be executed by one or more processors of an electronic device with a display and input device, the one or more programs including instructions for performing the method of Item A17.
   Item A29. A method for treatment of depressive symptoms of multiple sclerosis, in a subject, in need thereof, comprising administering to said subject a computerized method of any of Items A8-A16.
   Item A30. A method for treatment of depressive symptoms of multiple sclerosis, in a subject, in need thereof, comprising administering to said subject a computerized method of Item A17.
   Item A31. A method for treatment of depressive symptoms of multiple sclerosis, in a subject, in need thereof, comprising administering to said subject a digital therapeutic of any of Items A19-A26.
   Item B1. An electronic device for displaying feeling intensity inputs, the electronic device comprising:
a display;
an input device;
one or more processors; and
memory storing one or more programs configured to be executed by the one or more processors, the one or more programs including instructions for:
   generating, for display on the display, a journal entry, the journal entry indicating at least any difference between a first feeling intensity input and a second feeling intensity input.
      Item B2. The electronic device of Item B1, wherein the one or more programs also include instructions for:
displaying, on the display, a feeling selection interface, the feeling selection interface presenting a plurality of feeling interface elements, each feeling interface element being associated with a particular feeling; and
while displaying the feeling selection interface, receiving, via the input device, a first sequence of inputs, the first sequence of inputs including a feeling selection input, the feeling selection input corresponding to a particular feeling interface element.
   Item B3. The electronic device of any of Items B1-B2, wherein the one or more programs also include instructions for:
displaying, on the display, a feeling spectrum interface, the feeling spectrum interface presenting a plurality of intensities associated with the particular feeling; and
while displaying the feeling spectrum interface, receiving, via the input device, a second sequence of inputs, the second sequence of inputs including a first feeling intensity input, the first feeling intensity input corresponding to a first intensity of the plurality of intensities.
   Item B4. The electronic device of any of Items B1-B3, wherein the one or more programs also include instructions for:
displaying, on the display, an automatic thought selection interface, the automatic thought selection interface presenting a plurality of automatic thought interface elements, each automatic thought interface element being associated with a particular automatic thought; and
while displaying the automatic thought selection interface, receiving, via the input device, a third sequence of inputs, the third sequence of inputs including an automatic thought selection input, the automatic thought selection input corresponding to a particular automatic thought interface element.
   Item B5. The electronic device of any of Items B1-B4, wherein the one or more programs also include instructions for:
displaying, on the display, an alternative thought selection interface, the alternative thought selection interface presenting a plurality of alternative thought interface elements, each alternative thought interface element being associated with a particular alternative thought; and
while displaying the alternative thought selection interface, receiving, via the input device, a fourth sequence of inputs, the fourth sequence of inputs including an alternative thought selection input, the alternative thought selection input corresponding to a particular alternative thought interface element.
   Item B6. The electronic device any of Items B1-B5, wherein the journal entry is modified to further indicate one or more particular thinking trap interface elements.
   Item B7. The electronic device of Item B6, wherein the one or more programs also include instructions for:
in response to receiving the one or more thinking trap selection inputs, displaying, on the display, a quick recap interface element, the quick recap interface element indicating the particular automatic thought and the one or more particular thinking trap elements.
   Item B8. The electronic device of Item B7, wherein the journal entry is modified to further indicate the particular alternative thought interface element.
   Item B9. A computerized method for displaying feeling intensity inputs, the method comprising:
at an electronic device including a display and an input device:
   generating, for display on the display, a journal entry, the journal entry indicating at least any difference between the first feeling intensity input and the second feeling intensity input.
      Item B10. The computerized method of Item B9, wherein the method further comprising:
at the electronic device including a display and an input device:
   displaying, on the display, a feeling selection interface, the feeling selection interface presenting a plurality of feeling interface elements, each feeling interface element being associated with a particular feeling; and
   while displaying the feeling selection interface, receiving, via the input device, a first sequence of inputs, the first sequence of inputs including a feeling selection input, the feeling selection input corresponding to a particular feeling interface element.
      Item B11. The computerized method of any of Items B9-B10, wherein the method further comprising:
at the electronic device including a display and an input device:
   displaying, on the display, a feeling spectrum interface, the feeling spectrum interface presenting a plurality of intensities associated with the particular feeling; and
   while displaying the feeling spectrum interface, receiving, via the input device, a second sequence of inputs, the second sequence of inputs including a first feeling intensity input, the first feeling intensity input corresponding to a first intensity of the plurality of intensities.
      Item B12. The computerized method of any of Items B9-B11, wherein the method further comprising:
at the electronic device including a display and an input device:
   displaying, on the display, an automatic thought selection interface, the automatic thought selection interface presenting a plurality of automatic thought interface elements, each automatic thought interface element being associated with a particular automatic thought; and
   while displaying the automatic thought selection interface, receiving, via the input device, a third sequence of inputs, the third sequence of inputs including an automatic thought selection input, the automatic thought selection input corresponding to a particular automatic thought interface element.
      Item B13. The computerized method any of Items B9-B12, wherein the method further comprising:
at the electronic device including a display and an input device:
   displaying, on the display, an alternative thought selection interface, the alternative thought selection interface presenting a plurality of alternative thought interface elements, each alternative thought interface element being associated with a particular alternative thought;
   while displaying the alternative thought selection interface, receiving, via the input device, a fourth sequence of inputs, the fourth sequence of inputs including an alternative thought selection input, the alternative thought selection input corresponding to a particular alternative thought interface element.
      Item B14. The computerized method any of Items B9-B13, wherein the method further comprising:
at the electronic device including a display and an input device:
   displaying, on the display, the feeling spectrum interface; and
   while displaying the feeling spectrum interface, receiving, via the input device, a fifth sequence of inputs, the fifth sequence of inputs including a second feeling intensity input, the second feeling intensity input corresponding to a second intensity of the plurality of intensities.
      Item B15. The computerized method any of Items B9-B14, wherein the journal entry is modified to further indicate one or more particular thinking trap interface elements.
      Item B16. The computerized method any of Items B9-B15, wherein the method further comprising:
at the electronic device including a display and an input device:
   displaying, on the display, a quick recap interface element, the quick recap interface element indicating the particular automatic thought and the one or more particular thinking trap elements.
      Item B17. The computerized method any of Items B9-B16, wherein the journal entry is modified to further indicate the particular alternative thought interface element.
      Item B18. A computerized method for displaying feeling intensity inputs, the method comprising:
at an electronic device including a display and an input device:
   determining any difference between a first intensity and a second intensity to provide feeling intensity difference data; and
   displaying, on the display, the feeling intensity difference data.
      Item B19. The computerized method of Item B18, the method further comprising:
receiving, via the input device, feeling assessment data, the feeling assessment data describing a feeling associated with a user; and
receiving, via the input device, first feeling intensity data, the first feeling intensity data describing a first intensity of the feeling associated with the user.
   Item B20. The computerized method of any of Items B18-B19, the method further comprising:
identifying a plurality of potential automatic thoughts based on the feeling associated with the user, each potential automatic thought of the plurality of potential automatic thoughts corresponding to a negative thought; and
receiving, via the input device, automatic thought selection data, the automatic thought selection data identifying a particular potential automatic thought from among the plurality of potential automatic thoughts.
   Item B21. The computerized method of any of Items B18-B20, the method further comprising:
identifying a plurality of potential alternative thoughts based on the automatic thought selection data, each potential alternative thought of the plurality of potential alternative thoughts corresponding to a positive thought; and
receiving, via the input device, alternative thought selection data, the alternative thought selection data identifying a particular potential alternative thought from among the plurality of potential alternative thoughts.
   Item B22. An electronic device, comprising:
a display;
an input device;
one or more processors; and
   memory storing one or more programs configured to be executed by the one or more processors, the one or more programs including instructions for:
determining any difference between a first intensity and a second intensity to provide feeling intensity difference data; and
displaying, on the display, the feeling intensity difference data.
   Item B23. The electronic device of Item B22, wherein the one or more programs also include instructions for:
receiving, via the input device, feeling assessment data, the feeling assessment data describing a feeling associated with a user; and
receiving, via the input device, first feeling intensity data, the first feeling intensity data describing a first intensity of the feeling associated with the user.
   Item B24. The electronic device of any of Items B22-B23, wherein the one or more programs also include instructions for:
identifying a plurality of potential automatic thoughts based on the feeling associated with the user, each potential automatic thought of the plurality of potential automatic thoughts corresponding to a negative thought; and
receiving, via the input device, automatic thought selection data, the automatic thought selection data identifying a particular potential automatic thought from among the plurality of potential automatic thoughts.
   Item B25. The electronic device of any of Items B22-B24, wherein the one or more programs also include instructions for:
identifying a plurality of potential alternative thoughts based on the automatic thought selection data, each potential alternative thought of the plurality of potential alternative thoughts corresponding to a positive thought; and
receiving, via the input device, alternative thought selection data, the alternative thought selection data identifying a particular potential alternative thought from among the plurality of potential alternative thoughts.
   Item B26. A digital therapeutic for displaying feeling intensity inputs, the digital therapeutic comprising:
a display module, the display module configured to generate display data representing feeling intensity difference data.
   Item B27. The digital therapeutic of Item B26 further comprising:
an automatic thought identification module, the automatic thought identification module configured to (i) identify a plurality of potential automatic thoughts based on feeling assessment data describing a feeling associated with a user, each potential automatic thought of the plurality of potential automatic thoughts corresponding to a negative thought and (ii) receive automatic thought selection data identifying a particular potential automatic thought from among the plurality of potential automatic thoughts.
   Item B28. The digital therapeutic of any of Items B26-B27 further comprising:
an alternative thought identification module, the alternative thought identification module configured to (i) identify a plurality of potential alternative thoughts based on the automatic thought selection data, each potential alternative thought of the plurality of potential alternative thoughts corresponding to a positive thought and (ii) receive alternative thought selection data identifying a particular potential alternative thought from among the plurality of potential alternative thoughts.
   Item B29. The digital therapeutic of Items B26-B28 further comprising:
a feeling intensity module, the feeling intensity module configured to (i) receive first feeling intensity data describing a first intensity of the feeling associated with the user at a first point in time; (ii) receive second feeling intensity data describing a second intensity of the feeling associated with the user at a second point in time, the second point in time being later than the first point in time; and (iii) generate feeling intensity difference data, the feeling intensity difference data indicating any difference between the first intensity and the second intensity.
   Item B30. The digital therapeutic of Items B26-B29 further comprising:
a feeling assessment module, the feeling assessment module configured to receive the feeling assessment data describing the feeling associated with the user
   Item B31. The digital therapeutic of Items B26-B30 further comprising:
a thinking traps module, the thinking traps module configured to (i) identify a plurality of potential thinking traps based on the feeling assessment data and (ii) receive thinking trap selection data identifying one or more particular potential thinking traps from among the plurality of potential thinking traps.
   Item C1. An electronic device for treating depressive symptoms associated with multiple sclerosis, the electronic device comprising:
a display;
an input device;
one or more processors; and
memory storing one or more programs configured to be executed by the one or more processors, the one or more programs including instructions for:
displaying on the display a plurality of patient cognitive behavioral therapy screens that require a patient input, the therapy screens having thereon a patient selection or data input graphical user interface element operable by the patient, the display of a next therapy screen being dependent on the operation of the graphical user interface element on a presently displayed therapy screen.

### Example 2: Translational Study

| Study: | Translational |
|---|---|
| Primary Objectives | Primary Endpoint |
| To assess the feasibility of | Findings from interviews, |
| implementing the digital- | user satisfaction surveys |
| therapeutics application in an | and in-therapeutic software |
| outpatient clinic setting | application data |
| To evaluate ease of use and | Findings from interviews, |
| user satisfaction with the | user satisfaction surveys |
| digital-therapeutics | and in-therapeutic software |
| application | application data |
| To assess dosing of and | Patient use patterns of the digital- |
| engagement with the digital- | therapeutics application including |
| therapeutics application by | module completion, self-report |
| patients over a 4, 8 and 12- | assessment completion, voluntary |
| week period | use of self-report features, response |
| | to notifications |
| Secondary Objectives | Secondary Endpoint |
| To assess the safety of the | Rate of Treatment-Emergent- |
| digital-therapeutics | Adverse-Event (TEAE) related to |
| application | the digital-therapeutics application |
| To gain insight into | Absolute changes in scores from |
| preliminary efficacy of the | Baseline to the End of Study visit |
| digital-therapeutics | (EOS) of the Montgomery-Asberg |
| application | Depression Rating Scale (MADRS), |
| | Beck Depression Inventory-Second |
| | Edition (BDI-II), and the MS Quality |
| | of Life Inventory (MSQLI) |

### Example 3: Pivotal Study

| Study: | Pivotal |
|---|---|
| Primary Objectives | Endpoint(s) for Primary Objective(s) |
| To assess efficacy in | Changes in scores from Baseline to the End of |
| reducing depressive | Study visit (EOS) of the Montgomery-Asberg |
| symptoms | Depression Rating Scale (MADRS) |
| | Proportion of patients with 3 points |
| | improvement in MADRS vs. baseline and |
| | sham |
| Secondary Objectives | Endpoint(s) for Secondary Objective(s) |
| To assess efficacy in | Changes in MSQOL-54 > 5 SD |
| improving MS Quality | |
| of Life | |

### Patient Population:

Adults (18+) patients diagnosed with RRMS (McDonald Criteria) or CIS (Clinically Isolated Syndrome), who have depressive symptoms (e.g. MADRS total score ≥20) or major depressive disorder (DSM-5 diagnosis criteria).

Antidepressants allowed, but patients must be stable on antidepressant for 4 weeks or longer. Patients on DMT must be stable for two months or longer.

### Inclusion Criteria

Patients eligible for inclusion only if they meet all the criteria listed below. Patients may undergo rescreening following consultation with and approval of the Medical Monitor:
1. Provide written informed consent prior to any study specific assessments being performed
2. Male or female ≥18 years of age, inclusive
3. Proficient in English at 7th grade reading level or higher, in the judgement of the investigator
4. Diagnosed with Relapsing/Remitting MS (RRMS). Include patients with Clinically Isolated Syndrome (CIS)
5. If the subject is on antidepressant medication, must not have any antidepressant medication or dosage changes in the 4 weeks prior to screening
6. Screened positive for depression with a score ≥20 on the MADRS
7. Capable of using common software applications on a mobile device
8. Have access to a mobile device for the duration of the trial
9. Have not participated in any other investigational drug trials within the past 30 days (or within 5 half lives of study drug, whichever is longer) of enrollment
10. Considered appropriate for participation by their clinician

### Study Duration and Dosage (Digital Application) Regimen:

Daily to 3-4 times/week over the course of 12 weeks.

1. Use of a disease-modifying therapy for multiple sclerosis or an antidepressant, for the manufacture of a medicament for the treatment of depression associated with multiple sclerosis, wherein
i) the medicament is provided in combination with a computer-implemented device; and
ii) the computer-implemented device comprises:
a display;
an input device;
one or more processors; and
memory storing one or more programs configured to be executed by the one or more processors, the one or more programs including instructions for:
displaying, on the display, a feeling selection interface, the feeling selection interface presenting a plurality of feeling interface elements, each feeling interface element being associated with a particular feeling;
while displaying the feeling selection interface, receiving, via the input device, a first sequence of inputs, the first sequence of inputs including a feeling selection input, the feeling selection input corresponding to a particular feeling interface element;
in response to receiving the feeling selection input, displaying, on the display, a feeling spectrum interface, the feeling spectrum interface presenting a plurality of intensities associated with the particular feeling;
while displaying the feeling spectrum interface, receiving, via the input device, a second sequence of inputs, the second sequence of inputs including a first feeling intensity input, the first feeling intensity input corresponding to a first intensity of the plurality of intensities;
in response to receiving the first feeling intensity input, displaying, on the display, an automatic thought selection interface, the automatic thought selection interface presenting a plurality of automatic thought interface elements, each automatic thought interface element being associated with a particular automatic thought;
while displaying the automatic thought selection interface, receiving, via the input device, a third sequence of inputs, the third sequence of inputs including an automatic thought selection input, the automatic thought selection input corresponding to a particular automatic thought interface element;
in response to receiving the automatic thought selection input, displaying, on the display, an alternative thought selection interface, the alternative thought selection interface presenting a plurality of alternative thought interface elements, each alternative thought interface element being associated with a particular alternative thought;
while displaying the alternative thought selection interface, receiving, via the input device, a fourth sequence of inputs, the fourth sequence of inputs including an alternative thought selection input, the alternative thought selection input corresponding to a particular alternative thought interface element;
in response to receiving the alternative thought selection input, displaying, on the display, the feeling spectrum interface;
while displaying the feeling spectrum interface, receiving, via the input device, a fifth sequence of inputs, the fifth sequence of inputs including a second feeling intensity input, the second feeling intensity input corresponding to a second intensity of the plurality of intensities;
generating, for display on the display, a journal entry, the journal entry indicating at least any difference between the first feeling intensity input and the second feeling intensity input;
in response to receiving the automatic thought selection input, displaying, on the display, a thinking traps interface, the thinking traps interface presenting a plurality of thinking trap interface elements associated with the particular automatic thought interface element, each thinking trap interface element being associated with a particular thinking trap;
while displaying the thinking traps interface, receiving, via the input device, a sixth sequence of inputs, the sixth sequence of inputs including one or more thinking trap selection inputs, the one or more thinking trap selection inputs corresponding to one or more particular thinking trap interface elements; and
wherein the journal entry is modified to further indicate the one or more particular thinking trap interface elements.

2. Use of a disease-modifying therapy for multiple sclerosis, or an antidepressant, for the manufacture of a medicament for the treatment of depressive disorder associated with multiple sclerosis, wherein
i) the medicament is provided in combination with a computer-implemented device; and
ii) the computer-implemented device comprises:
a display;
an input device;
one or more processors; and
memory storing one or more programs configured to be executed by the one or more processors, the one or more programs including instructions for:
displaying, on the display, a feeling selection interface, the feeling selection interface presenting a plurality of feeling interface elements, each feeling interface element being associated with a particular feeling;
while displaying the feeling selection interface, receiving, via the input device, a first sequence of inputs, the first sequence of inputs including a feeling selection input, the feeling selection input corresponding to a particular feeling interface element;
in response to receiving the feeling selection input, displaying, on the display, a feeling spectrum interface, the feeling spectrum interface presenting a plurality of intensities associated with the particular feeling;
while displaying the feeling spectrum interface, receiving, via the input device, a second sequence of inputs, the second sequence of inputs including a first feeling intensity input, the first feeling intensity input corresponding to a first intensity of the plurality of intensities;
in response to receiving the first feeling intensity input, displaying, on the display, an automatic thought selection interface, the automatic thought selection interface presenting a plurality of automatic thought interface elements, each automatic thought interface element being associated with a particular automatic thought;
while displaying the automatic thought selection interface, receiving, via the input device, a third sequence of inputs, the third sequence of inputs including an automatic thought selection input, the automatic thought selection input corresponding to a particular automatic thought interface element;
in response to receiving the automatic thought selection input, displaying, on the display, an alternative thought selection interface, the alternative thought selection interface presenting a plurality of alternative thought interface elements, each alternative thought interface element being associated with a particular alternative thought;
while displaying the alternative thought selection interface, receiving, via the input device, a fourth sequence of inputs, the fourth sequence of inputs including an alternative thought selection input, the alternative thought selection input corresponding to a particular alternative thought interface element;
in response to receiving the alternative thought selection input, displaying, on the display, the feeling spectrum interface;
while displaying the feeling spectrum interface, receiving, via the input device, a fifth sequence of inputs, the fifth sequence of inputs including a second feeling intensity input, the second feeling intensity input corresponding to a second intensity of the plurality of intensities;
generating, for display on the display, a journal entry, the journal entry indicating at least any difference between the first feeling intensity input and the second feeling intensity input; and
in response to receiving the automatic thought selection input, displaying, on the display, a thinking traps interface, the thinking traps interface presenting a plurality of thinking trap interface elements associated with the particular automatic thought interface element, each thinking trap interface element being associated with a particular thinking trap;
while displaying the thinking traps interface, receiving, via the input device, a sixth sequence of inputs, the sixth sequence of inputs including one or more thinking trap selection inputs, the one or more thinking trap selection inputs corresponding to one or more particular thinking trap interface elements; and
wherein the journal entry is modified to further indicate the one or more particular thinking trap interface elements.

3. The use according to claim 1 or 2, wherein the computer-implemented device delivers behavioral therapy, such as cognitive behavioral therapy.

4. Use of a disease-modifying therapy for multiple sclerosis or an antidepressant, for the manufacture of a medicament for the treatment of depression associated with multiple sclerosis, wherein
i) the medicament is provided in combination with a digital-therapeutic; and
ii) the digital-therapeutic delivers behavioral therapy, such as cognitive behavioral therapy.

5. Use of a disease-modifying therapy for multiple sclerosis or an antidepressant, for the manufacture of a medicament for the treatment of depressive disorder associated with multiple sclerosis, wherein
i) the medicament is provided in combination with a digital-therapeutic; and
ii) the digital-therapeutic delivers behavioral therapy, such as cognitive behavioral therapy.

6. Use of a disease-modifying therapy for multiple sclerosis or an antidepressant, for the manufacture of a medicament for the treatment of depression associated with multiple sclerosis, wherein the use is combined with computer-implemented behavioral therapy, such as computer-implemented cognitive behavioral therapy.

7. Use of a disease-modifying therapy for multiple sclerosis or an antidepressant, for the manufacture of a medicament for the treatment of depressive disorder associated with multiple sclerosis, wherein the use is combined with computer-implemented behavioral therapy, such as computer-implemented cognitive behavioral therapy.

8. The use according to any one of claims 1 to 7, wherein the behavioral therapy is cognitive behavioral therapy for depression associated with multiple sclerosis or cognitive behavioral therapy for depressive disorder associated with multiple sclerosis, such as cognitive behavioral therapy for depression associated with multiple sclerosis that focuses on mood management, and coping with symptoms of multiple sclerosis or cognitive behavioral therapy for depressive disorder associated with multiple sclerosis that focuses on mood management and coping with symptoms of multiple sclerosis.

9. The use according to any one of claim 1, 4 or 6, wherein the treatment of depression associated with multiple sclerosis is characterized by the reduction of depressive symptoms, for example, wherein the depressive symptoms are one or more depressive symptoms selected from the group according to the Montgomery-Åsberg Depression Rating Scale, the Beck Depression Inventory (e.g. .BDI-II) or the Patient Healthy Questionnaire-9 (PHQ-9).

10. The use according to any one of claims 1 to 9, wherein multiple sclerosis is selected from relapsing-remitting multiple sclerosis (RRMS), primary-progressive multiple sclerosis (PPMS), secondary-progressive multiple sclerosis (SPMS), such as relapsing SPMS and non-relapsing SPMS, progressive-relapsing multiple sclerosis (PRMS), relapsing multiple sclerosis (RMS) and clinically isolated syndrome (CIS); in particular RRMS or CIS.

11. The use according to any one of claims 1 to 10, wherein the computer-implemented device is a computer-implemented mobile device, for example selected from the group consisting of a smartphone, a laptop computer, a tablet computer and a wearable computer; in particular a smartphone.

12. A combination comprising a disease-modifying therapy for multiple sclerosis, or an antidepressant and a computer-implemented device, wherein the computer-implemented device comprises:
a display;
an input device;
one or more processors; and
memory storing one or more programs configured to be executed by the one or more processors, the one or more programs including instructions for:
displaying, on the display, a feeling selection interface, the feeling selection interface presenting a plurality of feeling interface elements, each feeling interface element being associated with a particular feeling;
while displaying the feeling selection interface, receiving, via the input device, a first sequence of inputs, the first sequence of inputs including a feeling selection input, the feeling selection input corresponding to a particular feeling interface element;
in response to receiving the feeling selection input, displaying, on the display, a feeling spectrum interface, the feeling spectrum interface presenting a plurality of intensities associated with the particular feeling;
while displaying the feeling spectrum interface, receiving, via the input device, a second sequence of inputs, the second sequence of inputs including a first feeling intensity input, the first feeling intensity input corresponding to a first intensity of the plurality of intensities;
in response to receiving the first feeling intensity input, displaying, on the display, an automatic thought selection interface, the automatic thought selection interface presenting a plurality of automatic thought interface elements, each automatic thought interface element being associated with a particular automatic thought;
while displaying the automatic thought selection interface, receiving, via the input device, a third sequence of inputs, the third sequence of inputs including an automatic thought selection input, the automatic thought selection input corresponding to a particular automatic thought interface element;
in response to receiving the automatic thought selection input, displaying, on the display, an alternative thought selection interface, the alternative thought selection interface presenting a plurality of alternative thought interface elements, each alternative thought interface element being associated with a particular alternative thought;
while displaying the alternative thought selection interface, receiving, via the input device, a fourth sequence of inputs, the fourth sequence of inputs including an alternative thought selection input, the alternative thought selection input corresponding to a particular alternative thought interface element;
in response to receiving the alternative thought selection input, displaying, on the display, the feeling spectrum interface;
while displaying the feeling spectrum interface, receiving, via the input device, a fifth sequence of inputs, the fifth sequence of inputs including a second feeling intensity input, the second feeling intensity input corresponding to a second intensity of the plurality of intensities;
generating, for display on the display, a journal entry, the journal entry indicating at least any difference between the first feeling intensity input and the second feeling intensity input;
in response to receiving the automatic thought selection input, displaying, on the display, a thinking traps interface, the thinking traps interface presenting a plurality of thinking trap interface elements associated with the particular automatic thought interface element, each thinking trap interface element being associated with a particular thinking trap;
while displaying the thinking traps interface, receiving, via the input device, a sixth sequence of inputs, the sixth sequence of inputs including one or more thinking trap selection inputs, the one or more thinking trap selection inputs corresponding to one or more particular thinking trap interface elements; and
wherein the journal entry is modified to further indicate the one or more particular thinking trap interface elements.

13. The combination according to claim 12, wherein the computer-implemented device is a computer-implemented mobile device, for example selected from the group consisting of a smartphone, a laptop computer, a tablet computer and a wearable computer; in particular a smartphone.

14. A method for the treatment of depressive disorder associated with multiple sclerosis, in a subject, in need thereof, comprising administering to said subject behavioral therapy provided by a computer-implemented device comprising:
a display;
an input device;
one or more processors; and
memory storing one or more programs configured to be executed by the one or more processors, the one or more programs including instructions for:
displaying, on the display, a feeling selection interface, the feeling selection interface presenting a plurality of feeling interface elements, each feeling interface element being associated with a particular feeling;
while displaying the feeling selection interface, receiving, via the input device, a first sequence of inputs, the first sequence of inputs including a feeling selection input, the feeling selection input corresponding to a particular feeling interface element;
in response to receiving the feeling selection input, displaying, on the display, a feeling spectrum interface, the feeling spectrum interface presenting a plurality of intensities associated with the particular feeling;
while displaying the feeling spectrum interface, receiving, via the input device, a second sequence of inputs, the second sequence of inputs including a first feeling intensity input, the first feeling intensity input corresponding to a first intensity of the plurality of intensities;
in response to receiving the first feeling intensity input, displaying, on the display, an automatic thought selection interface, the automatic thought selection interface presenting a plurality of automatic thought interface elements, each automatic thought interface element being associated with a particular automatic thought;
while displaying the automatic thought selection interface, receiving, via the input device, a third sequence of inputs, the third sequence of inputs including an automatic thought selection input, the automatic thought selection input corresponding to a particular automatic thought interface element;
in response to receiving the automatic thought selection input, displaying, on the display, an alternative thought selection interface, the alternative thought selection interface presenting a plurality of alternative thought interface elements, each alternative thought interface element being associated with a particular alternative thought;
while displaying the alternative thought selection interface, receiving, via the input device, a fourth sequence of inputs, the fourth sequence of inputs including an alternative thought selection input, the alternative thought selection input corresponding to a particular alternative thought interface element;
in response to receiving the alternative thought selection input, displaying, on the display, the feeling spectrum interface;
while displaying the feeling spectrum interface, receiving, via the input device, a fifth sequence of inputs, the fifth sequence of inputs including a second feeling intensity input, the second feeling intensity input corresponding to a second intensity of the plurality of intensities;
generating, for display on the display, a journal entry, the journal entry indicating at least any difference between the first feeling intensity input and the second feeling intensity input; and
in response to receiving the automatic thought selection input, displaying, on the display, a thinking traps interface, the thinking traps interface presenting a plurality of thinking trap interface elements associated with the particular automatic thought interface element, each thinking trap interface element being associated with a particular thinking trap;
while displaying the thinking traps interface, receiving, via the input device, a sixth sequence of inputs, the sixth sequence of inputs including one or more thinking trap selection inputs, the one or more thinking trap selection inputs corresponding to one or more particular thinking trap interface elements; and
wherein the journal entry is modified to further indicate the one or more particular thinking trap interface elements.

15. The method according to claim 15, wherein the behavioral therapy is cognitive behavioral therapy, such as cognitive behavioral therapy that focuses on mood management or coping with symptoms of multiple sclerosis.

16. The method according to claim 14 or 15, wherein multiple sclerosis is selected from relapsing-remitting multiple sclerosis (RRMS), primary-progressive multiple sclerosis (PPMS), secondary-progressive multiple sclerosis (SPMS; such as relapsing SPMS and non-relapsing SPMS), progressive-relapsing multiple sclerosis (PRMS), relapsing multiple sclerosis (RMS) and clinically isolated syndrome (CIS); in particular RRMS or CIS.

17. The method according to claim 14, 15, or 16, wherein the one or more programs further include instructions for:
in response to receiving a mindfulness selection input, displaying, on the display, a mindfulness technique interface, the mindfulness technique interface presenting a plurality of mindfulness technique interface elements, each mindfulness technique interface element being associated with a particular mindfulness technique; and
in response to receiving a mindfulness technique selection input indicating selection of a mindfulness technique interface element corresponding to a particular mindfulness technique, displaying, on the display, mindfulness data corresponding to the particular mindfulness technique, the mindfulness data including at least one of audio, video, or interactive data.

18. The method according to claim 14, 15, 16, or 17, wherein the one or more programs further include instructions for:
in response to receiving a fatigue selection input, displaying, on the display, a fatigue type interface, the fatigue type interface presenting a plurality of fatigue type interface elements, each fatigue type interface element being associated with a particular fatigue type; and
in response to receiving a fatigue type selection input indicating selection of a fatigue type interface element corresponding to a particular fatigue type, displaying, on the display, fatigue type data corresponding to the particular fatigue type, the fatigue type data including at least one of audio, video, or interactive data.
